SEQUENCE LISTING (73 sequences)

SEQ ID NO: 1 - DNA, 22 bp, Artificial Sequence
  Description of Artificial Sequence  Synthetic Primer
ctgctgcctcccgtaggagtct

SEQ ID NO: 2 - DNA, 23 bp, Artificial Sequence
  Description of Artificial Sequence  Synthetic Primer
cgtattaccgcggctgctggcac

SEQ ID NO: 3 - DNA, 23 bp, Artificial Sequence
  Description of Artificial Sequence  Synthetic Primer
cgcccagtaattccgattaacgc

SEQ ID NO: 4 - DNA, 23 bp, Artificial Sequence
  Description of Artificial Sequence  Synthetic Primer
tggactaccagggtatctaatcc

SEQ ID NO: 5 - DNA, 23 bp, Artificial Sequence
  Description of Artificial Sequence  Synthetic Primer
gggttgcgctcgttgcgggactt

SEQ ID NO: 6 - DNA, 23 bp, Artificial Sequence
  Description of Artificial Sequence  Synthetic Primer
taaggaggtgatccaaccgcagg

SEQ ID NO: 7 - DNA, 23 bp, Artificial Sequence
  Description of Artificial Sequence  Synthetic Primer
ggttctttttcactcccctcgcc

SEQ ID NO: 8 - DNA, 23 bp, Artificial Sequence
  Description of Artificial Sequence  Synthetic Primer
gacccattatacaaaaggtacgc

SEQ ID NO: 9 - DNA, 23 bp, Artificial Sequence
  Description of Artificial Sequence  Synthetic Primer
gccccgttacatcttccgcgcag

SEQ ID NO: 10 - DNA, 23 bp, Artificial Sequence
  Description of Artificial Sequence  Synthetic Primer
cgacaaggaatttcgctacctta

SEQ ID NO: 11 - DNA, 22 bp, Artificial Sequence
  Description of Artificial Sequence  Synthetic Primer
cttacccgacaaggaatttcgc

SEQ ID NO: 12 - DNA, 23 bp, Artificial Sequence
  Description of Artificial Sequence  Synthetic Primer
gagccgacatcgaggtgccaaac

SEQ ID NO: 13 - DNA, 21 bp, Artificial Sequence
  Description of Artificial Sequence  Synthetic Primer
ggttaagcctcacggttcatt

SEQ ID NO: 14 - DNA, 14 bp, Artificial Sequence
  Description of Artificial Sequence  Synthetic Primer
ggaagcgcacggca

SEQ ID NO: 15 - DNA, 23 bp, Artificial Sequence
  Description of Artificial Sequence  Synthetic Primer
ccccttctcccgaagttacgggg

SEQ ID NO: 16 - DNA, 21 bp, Artificial Sequence
  Description of Artificial Sequence  Synthetic Primer
gtgagctattacgctttcttt

SEQ ID NO: 17 - DNA, 23 bp, Artificial Sequence
  Description of Artificial Sequence  Synthetic Primer
taccggccgtgcgtacttagaca

SEQ ID NO: 18 - DNA, 23 bp, Artificial Sequence
  Description of Artificial Sequence  Synthetic Primer
tgccctccaatggatcctcgtta

SEQ ID NO: 19 - DNA, 23 bp, Artificial Sequence
  Description of Artificial Sequence  Synthetic Primer
ctacggaaaccttgttacgactt

SEQ ID NO: 20 - DNA, 23 bp, Artificial Sequence
  Description of Artificial Sequence  Synthetic Primer
gagcactgggcagaaatcacatc

SEQ ID NO: 21 - DNA, 23 bp, Artificial Sequence
  Description of Artificial Sequence  Synthetic Primer
gtttcttttcctccgctgactaa

SEQ ID NO: 22 - DNA, 23 bp, Artificial Sequence
  Description of Artificial Sequence  Synthetic Primer
tcctcagccaagcacatacacca

SEQ ID NO: 23 - DNA, 1427 bp, Bacillus subtilis
  modified_base (554)..(873): N = A, C, G or T/U
gagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttntccggaattattgggcgtaaagggctcgcaggcggtttcttaagtctgatgtgaaagcccccggctcaaccggggagggtcattggaaactggggaacttgagtgcagaagaggagagtggaattccacgtgtngcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagtaacgcattnagcactccgcctggggagtacggtcgcaagactgaaactcaaaggaattgacgggggccgcacaagcggtggagcatgtggtttaattcgaagcaacgcgaagaaccttaccaggtcttgacatcctctgacaatcctagagataggacgtcttcgggggcagagtgacaggtggtgcatggttgtcgtcagctcgtgtcgtgagatgttgggttaagtcccgcaacgagcgcaaccctggatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacaccc

SEQ ID NO: 24 - DNA, 1544 bp, Bacillus anthracis
gtttgatcctggctcaggatgaacgctggcggcgtgcctaatacatgcaagtcgagcgaatggattaagagcttgctcttatgaagttagcggcggacgggtgagtaacacgtgggtaacctgcccataagactgggataactccgggaaaccggggctaataccggataacattttgaaccgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaacctttttggagccagccgcctaaggtgggacagatgattggggtgaagtcgtaacaaggtagccgtatcggaaggtgcggctggatcacctcctttct

SEQ ID NO: 25 - DNA, 1449 bp, Enterococcus faecalis
cgaacgctggcggcgtgcctaatacatgcaagtcgaacgcttctttcctcccgagtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgctgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagcc

SEQ ID NO: 26 - DNA, 1548 bp, Lactococcus lactis
tttatttgagagtttgatcctggctcaggacgaacgctggcggcgtgcctaatacatgcaagttgagcgctgaaggttggtacttgtaccgactggatgagcagcgaacgggtgagtaacgcgtggggaatctgcctttgagcgggggacaacatttggaaacgaatgctaataccgcataaaaactttaaacacaagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttggtagagaagaacgttggtgagagtggaaagctcatcaagtgacggtaactacccagaaagggacggctaactacgtgccagcagccgcggtaatacgtaggtcccgagcgttgtccggatttattgggcgtaaagcgagcgcaggtggtttattaagtctggtgtaaaaggcagtggctcaaccattgtatgcattggaaactggtagacttgagtgcaggagaggagagtggaattccatgtgtagcggtgaaatgcgtagatatatggaggaacaccggtggcgaaagcggctctctggcctgtaactgacactgaggctcgaaagcgtggggagcaaacaggattagataccctggtagtccacgccgtaaacgatgagtgctagatgtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtgaagtcgtaacaaggtagccgtatcggaaggtgcggctggatcacctccttt

SEQ ID NO: 27 - DNA, 1524 bp, Listeria monocytogenes
gcctgcaggtcgacaacagagtttgatcatggctcaggacgaacgctggcggcgtgcctaatacatgcaagtcgaacgaacggaggaagagcttgctcttccaaagttagtggcggacgggtgagtaacacgtgggcaacctgcctgtaagttggggataactccgggaaaccggggctaataccgaatgataaagtgtggcgcatgccacgcttttgaaagatggtttcggctatcgcttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactctgcctggggagtacgaccgcaaggttgaaactcaaaggaattgacgggggcccgcacaagcgtggagcatgtggtttaattcgaagcaacgcgaagaaccttaccaggtcttgacatcctttgaccactctggagacagagctttcccttcgggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccgaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgagtacgttcccgggccttgtacacaccgcccgtcacaccacgagagtttgtaacacccgaagtcggtagggtaacctttatggagccagccgccgaaggtgggacagataattggggtgaagtcgtaacaaggtaaa

SEQ ID NO: 28 - DNA, 1555 bp, Staphylococcus aureus
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtattgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttct

SEQ ID NO: 29 - DNA, 1551 bp, Streptococcus mutans
agagtttgatcctggctcaggacgaacgctggcggcgtgcctaatacatgcaagtgggacgcaaggaaacacactgtgcttgcacaccgtgttttcttgagtcgcgaacgggtgagtaacgcgtaggtaacctgcctattagcgggggataactattggaaacgatagctaataccgcataatattaattattgcatgataattgattgaaagatgcaagcgcatcactagtagatggacctgcgttgtattagctagttggtaaggtaagagcttaccaaggcgacgatacatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaagctctgttgtaagtcaagaacgtgtgtgagagtggaaagttcacacagtgacggtagcttaccagaaagggacggctaactacgtgccagcagccgcggtaatacgtaggtcccgagcgttgtccggatttattgggcgtaaagggagcgcaggcggtcaggaaagtctggagtaaaaggctatggctcaaccatagtgtgctctggaaactgtctgacttgagtgcagaaggggagagtggaattccatgtgtagcggtgaaatgcgtagatatatggaggaacaccagtggcgaaagcggctctctggtctgtcactgacgctgaggctcgaaagcgtgggtagcgaacaggattagataccctggtagtccacgccgtaaacgatgagtgctaggtgttaggccctttccggggcttagtgccggagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctattcttagagataggaagttacttcggtacatcggagacaggtggtgcatggttgtcgtcagctcgtgtcgtgagatgttgggttaagtcccgcaacgagcgcaacccttattgttagttgccatcattaagttgggcactctagcgagactgccggtaataaaccggaggaaggtggggatgacgtcaaatcatcatgccccttatgacctgggctacacacgtgctacaatggtcggtacaacgagttgcgagccggtgacggcaagctaatctctgaaagccgatctcagttcggattggaggctgcaactcgcctccatgaagtcggaatcgctagtaatcgcggatcagcacgccgcggtgaatacgttcccgggccttgtacacaccgcccgtcacaccacgagagtttgtaacacccgaagtcggtgaggtaaccttttaagggccaagccgcctaaggtgggatggatgattggggtgaagtcgtaacaaggtagccgtatcggaaggtgcggctggatcacctccttt

SEQ ID NO: 30 - DNA, 1515 bp, Streptococcus pneumoniae
atttgatcctggctcaggacgaacgctggcggcgtgcctaatacatgcaagtagaacgctgaaggaggagcttgcttctctggatgagttgcgaacgggtgagtaacgcgtaggtaacctgcctggtagcgggggataactattggaaacgatagctaataccgcataagagtggatgttgcatgacatttgcttaaaaggtgcacttgcatcactaccagatggacctgcgttgtattagctagttggtggggtaacggctcaccaaggcgacgatacatagccgacctgagagggtgatcggccacactgggactgagacacgkcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtaggctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgctgtaaacgatgagtgctaggtgttagaccctttccggggtttagtgccgtagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtcagccgtttgggaga

SEQ ID NO: 31 - DNA, 1335 bp, Streptococcus pyogenes
gaacgggtgagtaacgcgtaggtaacctacctcatagcgggggataactattggaaacgatagctaataccgcataagagagactaacgcatgttagtaatttaaaaggggcaattgctccactatgagatggacctgcgttgtattagctagttggtgaggtaaaggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgttagagaagaatgatggtgggagtggaaaatccaccaagtgacggtaactaaccagaaagggacggctaactacgtgccagcagccgcggtaatacgtaggtcccgagcgttgtccggatttattgggcgtaaagcgagcgcaggcggttttttaagtctgaagttaaaggcattggctcaaccaatgtacgctttggaaactggagaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccggagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgcccgctctagagatagagttttacttcggtacatcggtgacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggttggtacaacgagtcgcaagccggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccga

SEQ ID NO: 32 - DNA, 1465 bp, Mycobacterium avium
  modified_base (298)..(881): N = A, C, G or T/U
ggcggcgtgcttaacacatgcaagtcgaacggaaaggcctcttcggaggtactcgagtggcgaacgggtgagtaacacgtgggcaatctaccctgcacttcgggataagcctgggaaactgggtctaataccggataggacctcaagacgcatgtcttctggtggaaagcttttgcggtgtgggatgggcccgcggcctatcagcttgttggtggggtgacggcctaccaaggcgacgacgggtagccggcctgagagggtgtccggccacactgggactgagatacggcccagactnctacgggaggcagcagtggggaatattgcacaatgggcgcaagcctgatgcagcgacgccgcgtgggggatgacggccttcgggttgtaaacctctttcaccatcgacgaaggtccgggttttctcggattgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgngcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgncgtaaacggtgggtactaggtgtgggtttccttccttgggatccgtgccgtagctaacgcattaagtaccccgcctggggagtacggncgcaaggctaaaactcaaaggaattgacgggggnccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcgggtaatgccggggactcgtgagagactgccggggtcaactcggaggaaggtggggatgacgtcaagtcatcatgccccttatgtccagggcttcacacatgctacaatggccggtacaaagggctgcgatgccgtaaggttaagcgaatccttttaaagccggtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccagtggcctaacccttttgggagggagctgtcgaaggtgggatcggcgattgggacgaagtcgtaacaaggtagccg

SEQ ID NO: 33 - DNA, 1536 bp, Mycobacterium tuberculosis
tttgtttggagagtttgatcctggctcaggacgaacgctggcggcgtgcttaacacatgcaagtcgaacggaaaggtctcttcggagatactcgagtggcgaacgggtgagtaacacgtgggtgatctgccctgcacttcgggataagcctgggaaactgggtctaataccggataggaccacgggatgcatgtcttgtggtggaaagcgctttagcggtgtgggatgagcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcaccatcgacgaaggtccgggttctctcggattgacggtaggtggagaagaagcaccggccaactacgtgccagcagccgcggtaatacgtagggtgcgagcgttgtccggaattactgggcgtaaagagctcgtaggtggtttgtcgcgttgttcgtgaaatctcacggcttaactgtgagcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtccagggcttcacacatgctacaatggccggtacaaagggctgcgatgccgcgaggttaagcgaatccttaaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaccctcgggagggagctgtcgaaggtgggatcggcgattgggacgaagtcgtaacaaggtagccgtaccggaaggtgcggctggatcacctcctttct

SEQ ID NO: 34 - DNA, 1536 bp, Escherichia coli; identical to SEQ ID NO: 33

SEQ ID NO: 35 - DNA, 1534 bp, Klebsiella pneumoniae
  modified_base (11)..(12): N = A, C, G or T/U
agagtttgatnntggctcagattgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcgatgaggttaataacctcatcgattgacgttaccctgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttt

SEQ ID NO: 36 - DNA, 1485 bp, ACTINOBACCILUS ACTIN
  modified_base (208)..(1476): N = A, C, G or T/U
attgaagagtttgatcatggctcagattgaacgctggcggcaggcttaacacatgcaagtcggacggtagcaggagaaagcttgctttcttgctgacgagtggcggacgggtgagtaatgcttgggaatctgtcttatggagggggataacgacgggaaactgtcgctaataccgcgtagagtcgggagacgaaagtgcgggactttntggccgcatgccatgagatgagcccaagtgtgattaggtagttggtggggtaaaggcctaccaagccgacgatcgctagctggtctgagaggatggccagccacaccgggactgagacacggcccngactcctacgggaggcagcagtggggaatattgcgcaatgggggcaaccctgacgcagccatgccgcgtgaatgaagaaggccttcgggttgtaaagttctttcggtattgaggaaggttggtgtgttaatagcatgccaaattgacgttaaatacagaagaagcaccggctaactccgtgccagcagccgcggtaatacggggggtgcgagcgttaatcggaataactgggcgtaaagggcacgtaggcggacctttaagtgaggtgtgaaatccccgggcttaacctgggnattgcatttcatactgggggtctggagtactttngggagggntagaattccacgtgtagcggtgaaatgcgtagagatgtggaggaataccgaaggcgaaggcagccccttggggatgtactgacgctgatgtgcgaaagcgtggggagcaaacaggattagataccctggtagtccacgctgtaaacggtgtcgatttggggattggggtttagccctggtgcccgaagctaacgtgataaatcgaccgcctggggagtacggccgcaaggttaaaactcaaatgaattgacgggggcccgcacaagcggtggagcatgtggtttaattcgatgcaacgcgaagaaccttacctactcttgacatccgaagaagaactcagagatgggtttgtgccttagggagctttgagacaggtgctgcatggcngtcgtcagctcgtgttgtgaaatgttgggttaagtcccgcaacgagcgcaacccttatcctttgtggccagcgacgtggtcgggaactcaaaggagactgccggtgataaaccggaggaaggtggggatgacgtcaagtcatcatggcccttacgagtagggctacacacgtgctacaatggcgtatacagagggtaaccaaccagcgatggggagtgaatctcagaaagtgcgtctaagttcggattggagtctgcaactcgactccatgaagtcggaatcgctagtaatcgcgaatcagaatgttgcggtgaatacgttcccgggccttgtacacaccgcccgtcacaccatgggagtgggttgtaccagaagtggatagctgaaccgagagggtggcgtttaccacggtatgattcangactgggg

SEQ ID NO: 37 - DNA, 1487 bp, Haemophilus influenzae
  modified_base (1)..(1387): N = A, C, G or T/U
naattgaagagtttgatcatggctcagattgaacgctggcggcaggcttaacacatgcaagtcgaacggtagcaggagaaagcttgctttcttgctgacgagtggcggacgggtgagtaatgcttgggaatctggcttatggagggggataacgacgggaaactgtcgctaataccgcgtattatcggaagatgaaagtgcgggactgagaggccgcatgccataggatgagcccaagtgggattaggtagttggtggggtaaatgcctaccaagcctgcgatctctagctggtctgagaggatgaccagccacactggaactgagacacggtccagactcctacgggaggcagcagtggggaatattgcgcnatggggggaaccctgacgcagccatgccgcgtgaatgaagaaggccttcgggttgtaaagttctttcggtattgaggaaggttgatgtgttaatagcacatcaaattgacgttaaatacagaagaagcaccggctaactccgtgccagcagccgcggtaatacggagngtgcgagcgttaatcggaataactgggcgtaaagggcacgcaggcggttatttaagtgaggtgtgaaagccccgggcttaacctgggnattgcatttcagactgggtaactagagtactttagggaggggtagaattccacgtgtagcggtgaaatgcgtagagatgtggaggaataccgaaggcgaaggcagccccttgggaatgtactgacgctcatgtgcgaaagcgtggggagcaaacaggattagataccctggtagtccacgctgtaaacgctgtcgatttgggggttggggtttaactctggcacccgtagctaacgtgataaatcgaccgcctggggagtacggccgcaaggttaaaactcaaatgaattgacgggggccngcacaagcggtggagcatgtggtttaattcgatgcaacgcgaagaaccttacctactcttgacatcctaagaagagctcagagatgagcttgtgccttcgggaacttagagacaggtgctgcatggctgtcgtcagctcgtgttgtgaaatgttgggttaagtcccgcaacgagcgcaacccttatcctttgttgccagcgacttggtcgggaactcaaaggagactgccagtgataaactggaggaaggtngggatgacgtcaagtcatcatggcccttacgagtagggctacacacgtgctacaatggcgtatacagagggaagcgaagctgcgaggtggagcgaatctcataaagtacgtctaagtccggattggagtctgcaactcgactccatgaagtcggaatcgctagtaatcgcgaatcagaatgtcgcggtgaatacgttcccgggcnttgtacacaccgcccgtcacaccatgggagtgggttgtaccagaagtagatagcttaaccttttggagggcgtttaccacggtatgattcatgactgggg

SEQ ID NO: 38 - DNA, 1532 bp, Bordetella bronchiseptica
tgaactgaagagtttgatcctggctcagattgaacgctggcgggatgctttacacatgcaagtcggacggcagcacgggcttcggcctggtggcgagtggcgaacgggtgagtaatgtatcggaacgtgcccagtagcgggggataactacgcgaaagcgtggctaataccgcatacgccctacgggggaaagcgggggaccttcgggcctcgcactattggagcggccgatatcggattagctagttggtggggtaacggcctaccaaggcgacgatccgtagctggtttgagaggacgaccagccacactgggactgagacacggcccagactcctacgggaggcagcagtggggaattttggacaatgggggcaaccctgatccagccatcccgcgtgtgcgatgaaggccttcgggttgtaaagcacttttggcaggaaagaaacggcacgggctaatatcctgtgcaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagggcttaaccttggaactgcatttttaactaccgggctagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactagctgttggggccttcgggccttggtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaatcccgaagagatttgggagtgctcgcaagagaaccggaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtcgggacagagggtcgccaacccgcgagggggagccaatcccagaaacccgatcgtagtccggatcgcagtctgcaactcgactgcgtgaagtcggaatcgctagtaatcgcggatcagcatgtcgcggtgaatacgttcccgggtcttgtacacaccgcccgtcacaccatgggagtgggttttaccagaagtagttagcctaaccgcaaggggggcgattaccacggtaggattcatgactggggtgaagtcgtaacaaggtagccgtatcggaaggtgcggctggatcacctccttt

SEQ ID NO: 39 - DNA, 1485 bp, Bordetella parapertussis
attgaacgctggcgggatgctttacacatgcaagtcggacggcagcacgggcttcggcctggtggcgagtggcgaacgggtgagtaatgtatcggaacgtgcccagtagcgggggataactacgcgaaagcgtggctaataccgcatacgccctacgggggaaagcgggggactttcgggcctcgcactattggagcggccgatatcggattagctagttggtggggtaacggcctaccaaggcgacgatccgtagctggtttgagaggacgaccagccacactgggactgagacacggcccagactcctacgggaggcagcagtggggaattttggacaatgggggcaaccctgatccagccatcccgcgtgtgcgatgaaggccttcgggttgtaaagcacttttggcaggaaagaaacggcacgggctaatatcctgtgcaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagggcttaaccttggaactgcatttttaactaccgggctagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactagctgttggggccttcgggccttggtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaatcccgaagagatttgggagtgctcgcaagagaaccggaacacaggtgctgcatggctgtcgtcagctcgtgtcgtgagatgttgggttaagtcccgcaacgagcgcaacccttgtcattagttgctacgaaagggcactctaatgagactgccggttacaaaccggaggaaggtggggatgacgtcaagtcctcatggcccttatgggtagggcttcacacgtcatacaatggtcgggacagagggtcgccaacccgcgagggggagccaatcccagaaacccgatcgtagtccggatcgcagtctgcaactcgactgcgtgaagtcggaatcgctagtaatcgcggatcagcatgtcgcggtgaatacgttcccgggtcttgtacacaccgcccgtcacaccatgggagtgggttttaccagaagtagttagcctaaccgcaaggggggggcgattaccacggtaggattcatgactggggtgaagtcgtaacaaggtagccgtatcggaagg

SEQ ID NO: 40 - DNA, 1464 bp, Bordetella pertussis
  modified_base (87)..(1391): N = A, C, G or T/U
aactgaagagtttgatcctggctcagattgaacgctggcgggatgctttacacatgcaagtcggacggcagcacgggcttcggcctngtggcgagtggcgaacgggtgagtaatgtatcggaacgtgcccagtagcgggggataactacgcgaaagcgtagctaataccgcatacgccctacgggggaaagcgggggaccttcgggcctcgcactattggagcggccgatatcggattagctngttggtggggtaacggcctaccaaggcgacgatccgtagctggtttgagaggacgaccagccacactgggactgagacacggcccagnctcctacgggaggcagcagtggggaattttggacaatgggggcaaccctgatccagccatcccgcgtgtgcgatgaaggccttcgggttgtaaagcacttttggcaggaaagaaacggcacgggctaatatcctgtgcaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagggcttaaccttggaactgcatttttaactaccgggctagagtgtgtcagagggaggtggaattccgcgtgtagcagtgaaatgcgtagatatgcggaggaacaccgatggcgaaggcagcctcctgggataacactgacgctcatgcacgaaagtgtggggagcaaacaggattagataccctggtagtccacgccctaaacgatgtcaactagctgttggggccttcgggccttggtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaatcccgaagagatttgggagtgctcgcaagagaaccggaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtgaagtcctcatggcccttatgggtagggcttcacacgtcatacaatggtcgggacagagggttgncaacccgcgagggggagccaatcccagaaacccggtcgtngtccggatcgcagtctgcaactcgactgcgtgaagtcggaatcgctagtaatcgcggatcagcatgtcgcggtgaatacgttcccgggtcttgtacacaccgcncgtcacaccatgggagtgggttttaccagaagtagttagcctaaccgcaaggggggcgattaccacggtagga

SEQ ID NO: 41 - DNA, 1535 bp, Burkholderia cepacia
taaactgaagagtttgatcctggctcagattgaacgctggcggcatgcttaacacatgcaagtcgaacggcagcacgggtgcttgcacctggtggcgagtggcgaacgggtgagtaatacatcggaacatgtcctgtagtgggggatagcccggcgaaagccggattaataccgcatacgatctacggatgaaagcgggggaccttcgggcctcgcgctatagggttggcgatggctgattagctagttggtggggtaaaggcctaccaaggcgacgatcagtagctggtctgagaggacgaccagccacactgggactgagacacggcccagactcctacgggaggcagcagtggggaattttggacaatgggcgaaagcctgatccagcaatgccgcgtgtgtgaagaaggccttcgggttgtaaagcacttttgtccggaaagaaatccctggctctaatacagtcgggggatgacggtaccggaagaataagcaccggctaactacgtgccagcagccgcggtaatacgtagggtgcaagcgttaatcggaattactgggcgtaaagcgtgcgcaggcggtttgctaagaccgatgtgaaatccccgggctcaacctgggaactgcattggtgactggcaggctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatggtcggaatcctgctgagaggtgggagtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggttgccaacccgcgagggggagctaatcccagaaaacccatcgtagtccggattgcactctgcaactcgagtgcatgaagctggaatcgctagtaatcgcggatcagcatgccgcggtgaatacgttcccgggtcttgtacacaccgcccgtcacaccatgggagtgggttttaccagaagtggctagtctaaccgcaaggaggacggtcaccacggtaggattcatgactggggtgaagtcgtaacaaggtagccgtatcggaaggtgcggctggatcacctcctttct

SEQ ID NO: 42 - DNA, 1488 bp, Burkholderia mallei
agattgaacgctggcggcatgccttacacatgcaagtcgaacggcagcacgggcttcggcctggtggcgagtggtgaacgggtgagtaatacatcggaacatgtcctgtagtgggggatagcccggcgaaagccggattaataccgcatacgatctgaggatgaaagcgggggaccttcgggcctcgcgctatagggttggccgatggctgattagctagttggtggggtaaaggcctaccaaggcgacgatcagtagctggtctgagaggacgaccagccacactgggactgagacacggcccagactcctacgggaggcagcagtggggaattttggacaatgggcgcaagcctgatccagcaatgccgcgtgtgtgaagaaggccttcgggttgtaaagcacttttgtccggaaagaaatcattctggctaatacccggagtggatgacggtaccggaagaataagcaccggctaactacgtgccagcagccgcggtaatacgtagggtgcgagcgttaattggaattactgggcgtaaagcgtgcgcaggcggtttgctaagaccgatgtgaaatccccgggctcaacctgggaactgcattggtgactggcaggctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatggtcggaagcccgatgagagttgggcgtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggtcgccaacccgcgagggggagccaatcccagaaaaccgatcgtagtccggattgcactctgcaactcgagtgcatgaagctggaatcgctagtaatcgcggatcagcatgccgcggtgaatacgttcccgggtcttgtacacaccgcccgtcacaccatgggagtgggttttaccagaagtggctagtctaaccgcaaggaggacggtcaccacggtaggattcatgactggggtgaagtcgtaacaaggtagccgtatcggaaggtgc

SEQ ID NO: 43 - DNA, 1610 bp, Burkholderia pseudomallei
tctagatgcgtgctcgagcggccgcccagtgctgcatggatatctgctgaattcggcttgagcagtttgatcctggctcagattgaacgctggcggcatgccttacacatgcaagtcgaacggcagcacgggcttcggcctggtggcgagtggcgaacgggtgagttatacatcggagcatgtcctgtagtgggggatagcccggcgaaagccgaattaataccgcatacgatctgaggatgaaagcgggggaccttcgggcctcgcgctatagggttggccgatggctgattagctagttggtggggtaaaggcctaccaaggcgacgatcagtagctggtctgagaggacgaccagccacactgggactgagacacggcccagactcctacgggaggcagcagtggggaattttggacaatgggcgcaagcctgatccagcaatgccgcgtgtgtgaagaaggccttcgggttgtaaagcacttttgtccggaaagaaatcattctggctaatacccggagtggatgacggtaccggaagaataagcaccggctaactacgtgccagcagccgcggtaatacgtagggtgcgagcgttaatcgggattactgggcgtaaagcgtgcgcaggcggtttgctaagaccgatgtgaaatccccgggctcaacctgggaactgcattggtgactggcaggctagagtatggcagaggggggtagaattccacgtgtagcagtgaaatgcgtagagatgtggaggaataccgatggcgaaggcagccccctgggccaatactgacgctcatgcacgaaagcgtggggagaaaacaggattagataccctggtagtccacgccctaaacgatgtcaactagttgttggggattcatttccttagtaacgtagctaacgcgcgaagttgaccgcctggggagtacggtcgcaagattaaaactcaaaggaattgacggggacccgcacaagcggtggatgatgtggattaattcgatgcaacgcgaaaaaccttacctacccttgacatggtcggaagcccgatgagagttgggcgtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggtcgccaacccgcgagggggagccaatcccagaaaaccgatcgtagtccggattgcactctgcaactcgagtgcatgaagctggaatcgctagtaatcgcggatcagcatgccgcggtgaatacgttcccgggtcttgtacacaccgcccgtcacaccatgggagtgggttttaccagaagtggctagtctaaccgcaaggaggacggtcaccacggtaggattcatgactggggtgaagtcgtaacaaggtagccgtagaagccgaattccagcacactggcggccgttactactggatccgagctcgtacc

SEQ ID NO: 44 - DNA, 1544 bp, Neisseria gonorrhoeae
tgaacataagagtttgatcctggctcagattgaacgctggcggcatgctttacacatgcaagtcggacggcagcacagggaagcttgcttctcgggtggcgagtggcgaacgggtgagtaacatatcggaacgtaccgggtagcgggggataactgatcgaaagatcagctaataccgcatacgtcttgagagggaaagcaggggaccttcgggccttgcgctatccgagcggccgatatctgattagctggttggcggggtaaaggcccaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgtcagggaagaaaaggctgttgccaatatcggcggccgatgacggtacctgaagaataagcaccggctaactacgtgccagcagccgcggtaatacgtagggtgcgagcgttaatcggaattactgggcgtaaagcgggcgcagacggttacttaagcaggatgtgaaatccccgggctcaacccgggaactgcgttctgaactgggtgactcgagtgtgtcagagggaggtggaattccacgtgtagcagtgaaatgcgtagagatgtggaggaataccgatggcgaaggcagcctcctgggataacactgacgttcatgtccgaaagcgtgggtagcaaacaggattagataccctggtagtccacgccctaaacgatgtcaattagctgttgggcaacttgattgcttggtagcgtagctaacgcgtgaaattgaccgcctggggagtacggtcgcaagattaaaactcaaaggaattgacggggacccgcacaagcggtggatgatgtggattaattcgatgcaacgcgaagaaccttacctggttttgacatgtgcggaatcctccggagacggaggagtgccttcgggagccgtaacacaggtgctgcatggctgtcgtcagctcgtgtcgtgagatgttgggttaagtcccgcaacgagcgcaacccttgtcattagttgccatcattcggttgggcactctaatgagactgccggtgacaagccggaggaaggtggggatgacgtcaagtcctcatggcccttatgaccagggcttcacacgtcatacaatggtcggtacagagggtagccaagccgcgaggcggagccaatctcacaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgcaaggagtccgcttaccacggtatgcttcatgactggggtgaagtcgtaacaaggtagccgtaggggaacctgcggctggatcacctcctttct

SEQ ID NO: 45 - DNA, 1544 bp, Neisseria meningitidis
tgaacataagagtttgatcctggctcagattgaacgctggcggcatgctttacacatgcaagtcggacggcagcacagagaagcttgcttctcgggtggcgagtggcgaacgggtgagtaacatatcggaacgtaccgagtagtgggggataactgatcgaaagatcagctaataccgcatacgtcttgagagagaaagcaggggaccttcgggccttgcgctattcgagcggccgatatctgattagctagttggtggggtaaaggcctaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgtcagggaagaaaaggctgttgctaatatcagcggctgatgacggtacctgaagaataagcaccggctaactacgtgccagcagccgcggtaatacgtagggtgcgagcgttaatcggaattactgggcgtaaagcgggcgcagacggttacttaagcaggatgtgaaatccccgggctcaacccgggaactgcgttctgaactgggtgactcgagtgtgtcagagggaggtagaattccacgtgtagcagtgaaatgcgtagagatgtggaggaataccgatggcgaaggcagcctcctgggacaacactgacgttcatgcccgaaagcgtgggtagcaaacaggattagataccctggtagtccacgccctaaacgatgtcaattagctgttgggcaacctgattgcttggtagcgtagctaacgcgtgaaattgaccgcctggggagtacggtcgcaagattaaaactcaaaggaattgacggggacccgcacaagcggtggatgatgtggattaattcgatgcaacgcgaagaaccttacctggtcttgacatgtacggaatcctccggagacggaggagtgccttcgggagccgtaacacaggtgctgcatggctgtcgtcagctcgtgtcgtgagatgttgggttaagtcccgcaacgagcgcaacccttgtcattagttgccatcattcagttgggcactctaatgagactgccggtgacaagccggaggaaggtggggatgacgtcaagtcctcatggcccttatgaccagggcttcacacgtcatacaatggtcggtacagagggtagccaagccgcgaggcggagccaatctcacaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtaggataaccacaaggagtccgcttaccacggtatgcttcatgactggggtgaagtcgtaacaaggtagccgtaggggaacctgcggctggatcacctcctttct

SEQ ID NO: 46 - DNA, 1537 bp, Pseudomonas aeruginosa
gaactgaagagtttgatcatggctcagattgaacgctggcagcaggggccttcaacacatgcaagtcgagcttatgaagggagcttgccttggattcagcggcggacgggtgagtaatgcctaggaatctgcctggtagtgggggataacgtccggaaacggccgctaataccgcatacgtcctgagggagaaagtcggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgcaagcctgatccagccatgccgcgtgtgtgaagaaggtcttcggattgtaaagcactttaagttgggaggaagggcagtaagttaataccttgctgtttgacgttaccaacagaataagcaccggctaacttcgtgccagcagccgcggtaatacgaagggtgcaagcgttaatcggaattactgggcgtaaagcgcgcgtaagtggttcagcaagcttgatgtgaaatccccgggctcaacctgggaactgcatccaaaagctactgagctagagtacggtagaggtggtagaatttcctgtgtagcggtgaaatgcgtagatataggaaggaacaccagtggcgaaggcgaccacctggactgtactgacactgaggtgcgaaagcgtggggagcaaacaggattagataccctggtagtccacgccgtaaacgatgtcgactagccgttgggatccttgagatcttagtggcgcacgtaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaacagagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgagtgggagctaatcccataaaaccgatcgtagtccggatcgcagtctgcaactcgactgcgtgaagtcggaatcgctagtaatcgtgaatcagaatgtcacggtgaatacgtccccgggccttgtacacaccgcccgtcacaccatgggagtgggttgctccagaagtagctagtctaaccgcaagggggacggttaccacggagtgattcatgactggggtgaagtcgtaacaaggtagccgtaggggaacctgcggctggatcacctcctta

SEQ ID NO: 47 - DNA, 1467 bp, Vibrio cholerae
  modified_base (928)..(1464): N = A, C, G or T/U
attgaagagtttgatcctggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgcaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtatgaagaaggccttcgggttgtaaagtactttcagtagggaggaaggtggttaagttaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgccctagagtcgtggctttcggagctaacgcgttaagtagaccgcctggggagtacggtcgcaagattaaaactcaaatgaattgacgggggnccgcacaagcggtggagcatgtggtttaattcganncaacgcgaagaaccttacctactcttgacatccagagaatctagcggagacgctggagtgccttcgggagctctgagacaggtgctgcatggctgtcgtcagctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccatgggagtgggctgcaaaagaagcangtagtttaaccttcgggaggacgcttnccc

SEQ ID NO: 48 - DNA, 1485 bp, Yersinia enterocolitica
  modified_base (1)..(1484): N = A, C, G or T/U
naattgaagagtttgatcatggctcagatngaacgctggcggcaggcctaacacatgcaagtcgagcggcagcgggaagnagtttactactttcngggcgagcggcgnacgggtgagtaatgtctgggaaactgcctgatggagggggataactactggaaacggtagctaataccgcataacgtcttcggaccaaagtgggggaccttagggcctcacgccatcngatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaaggccaataacttaatacgttgttggattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggnacngcatttgaaactggcaagctagagtcttgtagaggggggtagaattccaggtgtagcggtgaaatgcgtagagatctgnaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattnncgggggccngcacaagcggtggagcatgtggtttaattcgatgcaacgcgaagaaccttacctactcttgacatccacggaatttagcagagatgctttagtgncttcgggaaccgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcagatacaaagtgaagcgaactcgcgagagcaagcggaccacataaagtctgtcgtagtccggattggagtctgcaactcgactccatgaagtcggaatcgctagtaatcgtagatcagaatgctacggtgaatacgttcccgggccttgtacacaccgcccgtcacaccntgggagtgggttgcaaaagaagtaggtagcttaacnttcgggagggcgcgtaccactttgtgattcnngnc

SEQ ID NO: 49 - DNA, 2927 bp, Bacillus subtilis
ggttaagttagaaagggcgcacggtggatgccttggcactaggagccgatgaaggacgggacgaacaccgatatgcttcggggagctgtaagcaagctttgatccggagatttccgaatggggaaacccaccactcgtaatggagtggtatccatatctgaattcataggatatgagaaggcagacccggggaactgaaacatctaagtacccggagaagagaaagcaaatgcgattccctgagtagcggcgacgaacacgggatcagcccaaaccaagaggcttgcctctgtggttgtaggacactctgtacggagttacaaaagaacgaggtagatgaagaggtctggaaagggcccgccataggaggtaacagccctgtagtcaaaacttcgttctctcctgagtggatcctgagtacggcggaacacgtgaaattccgtcggaatccgggaggaccatctcccaaggctaaatactccctagtgaccgatagtgaaccagtaccgtgagggaaaggtgaaaagcaccccggaaggggagtgaaagagatcctgaaaccgtgtgcctacaagtagtcagagcccgttaacggtgatggcgtgccttttgtagaatgaaccggcgagttacgatcccgtgcaaggttaagcagaagatgcggagccgcagcgaaagcgagtctgaatagggcgcatgagtacgtggtcgtagacccgaaaccaggtgatctacccatgtccagggtgaagttcaggtaacactgaatggaggcccgaacccacgcacgttgaaaagtgcggggatgaggtgtgggtaggggtgaaatgccaatcgaacctggagatagctggttctctccgaaatagctttagggctagcctcaaggtaagagtcttggaggtagagcactgattggactaggggccctcaccgggttaccgaattcagtcaaactccgaatgccaatgacttatccttgggagtcagactgcgagtgataagatccgtagtcgaaagggaaacagcccagaccgccagctaaggtcccaaagtatacgttaagtggaaaaggatgtggagttgcttagacaaccaggatgttggcttagaagcagccaccatttaaagagtgcgtaatagctcactggtcgagtgactctgcgccgaaaatgtaccggggctaaacgtatcaccgaagctgcggactgttcttcgaacagtggtaggagagcgttctaagggctgtgaagccagaccggaaggactggtggacggcttagaagtgagaatgccggtatgagtagcgaaaagaggggtgagaatccctccaccgaatgcctaagggttcctgaggaaggctcgtccgctcagggttagtcgggacctaagccgaggccgaaaggcgtaggcgatggacaacaggttgatattcctgtaccacctcctcaccatttgagcaatggggggtcgcaggaggatagggtaagcgcggtattggatatccgcgtccaagcagttaggctgggaaataggcaaatccgtttcccataaggctgagctgtgatggcgagcgaaatatagtagcgaagttcctgattccacactgccaagaaaagcctctagcgaggtgagaggtgcccgtaccgcaaaccgtcacaggtaggcgaggagagaatcctaaggtgatcgagagaactctcgttaaggaactcggcaaaatgaccccgtaacttcgggagaaggggtgctctgttagggtgcaagcccgagagagccgcagtgaataggcccaggcgactgtttagcaaaaacacaggtctctgcgaagccgtaaggcgaagtataggggctgacgcctgcccggtgctggaaggttaagaggagcgcttagcgtaagcgaaggtgcgaattgaagccccagtaaacggcggccgtaactataacggtcctaaggtagcgaaattccttgtcgggtaagttccgacccgcacgaaaggcgcaacgatctgggcgctgtctcaacgagagactcggtgaaattatagtacctgtgaagatgcaggttacccgcgacaggacggaaagaccccgtggagctttactgcagcctgatattgaatgttggtacagcttgtacaggataggtaggagccttggaaaccggagcgccagcttcggtggaggcatcggtgggatactaccctggctgtattgaccttctaaccccccgcccttatcgggcggggagacagtgtcaggtgggcagtttgactggggcggtcgcctcctaaaaggtaacggaggcgcccaaaggttccctcagaatggttggaaatcattcgcagagtgtaaaggcacaagggagcttgactgcgagacctacaagtcgagcagggacgaaagtcgggcttagtgatccggtggttccgcatggaagggccatcgctcaacggataaaagctaccccggggataacaggcttatctcccccaagagctccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgctggaaatttgagaggagctgtccttagtacgagaggaccgggatggacgcaccgctggtgtaccagttgttctgccaagggcatcgctgggtagctatgtgcggacgggataagtgctgaaagcatctaagcatgaagcccccctcaagatgagatttcccattccgcaaggaagtaagatccctgaaagatgatcaggttgataggtctgaggtggaagtgtggcaacacatggagctgacagatactaatcgatcgaggacttaaccat

SEQ ID NO: 50 - DNA, 2922 bp, Bacillus anthracis
ggttaagttagaaagggcgcacggtggatgccttgacactaggagtcgatgaaggacgggactaacgccgatatgcttcggggagctgtaagtaagctttgatccgaagatttccgaatggggaaacccaccatacgtaatggtatggtatccttatctgaatacatagggtaaggaagacagacccagggaactgaaacatctaagtacctggaggaagagaaagcaaatgcgatttcctgagtagcggcgagcgaaacggaacatagcccaaaccaagaggcttgcctcttggggttgtaggacattctatacggagttacaaaggaacgaggtagacgaagcgacctggaaaggtccgtcgtagagggtaacaaccccgtagtcgaaacttcgttctctcttgaatgtatcctgagtacggcggaacacgtgaaattccgtcggaatctgggaggaccatctcccaaggctaaatactccctagtgatcgatagtgaaccagtaccgtgagggaaaggtgaaaagcaccccggaaggggagtgaaagagatcctgaaaccgtgtgcctacaaatagtcagagcccgttaacgggtgatggcgtgccttttgtagaatgaaccggcgagttacgatcccgtgcgaggttaagctgaagaggcggagccgcagcgaaagcgagtctgaatagggcgtttagtacgtggtcgtagacccgaaaccaggtgatctacccatgtccagggtgaagttcaggtaacactgaatggaggcccgaacccacgcacgttgaaaagtgcggggatgaggtgtgggtagcggagaaattccaatcgaacctggagatagctggttctccccgaaatagctttagggctagccttaagtgtaagagtcttggaggtagagcactgattggactaggggtcctcatcggattaccgaattcagtcaaactccgaatgccaatgacttatccttaggagtcagactgcgagtgataagatccgtagtcaaaagggaaacagcccagaccgccagctaaggtcccaaagtgtgtattaagtggaaaaggatgtggagttgcttagacaactaggatgttggcttagaagcagccaccatttaaagagtgcgtaatagctcactagtcgagtgactctgcgccgaaaatgtaccggggctaaatacaccaccgaagctgcggattgataccaatggtatcagtggtaggggagcgttctaaggacagtgaagtcagaccggaaggactggtggagtgcttagaagtgagaatgccggtatgagtagcgaaagacgggtgagaatcccgtccaccgaatgcctaaggtttcctgaggaaggctcgtccgctcagggttagtcaggacctaagccgaggccgacaggcgtaggcgatggacaacaggttgatattcctgtaccacctctttatcgtttgagcaatggagggacgcagaaggatagaagaagcgtgcgattggttgtgcacgtccaagcagttaggctgataagtaggcaaatccgcttatcgtgaaggctgagctgtgatggggaagctccttatggagcgaagtctttgattccccgctgccaagaaaagcttctagcgagataaaaggtgcctgtaccgcaaaccgacacaggtaggcgaggagagaatcctaaggtgtgcgagagaactctggttaaggaactcggcaaaatgaccccgtaacttcgggagaaggggtgctttcttaacggaaagccgcagtgaataggcccaagcgactgtttagcaaaaacacagctctctgcgaagccgtaaggcgaagtatagggggtgacacctgcccggtgctggaaggttaaggagaggggttagcgtaagcgaagctctgaactgaagccccagtaaacggcggccgtaactataacggtcctaaggtagcgaaattccttgtcgggtaagttccgacccgcacgaaaggtgtaacgatttgggcactgtctcaaccagagactcggtgaaattatagtacctgtgaagatgcaggttacccgcgacaggacggaaagaccccgtggagctttactgtagcctgatattgaattttggtacagtttgtacaggataggcgggagcctttgaaaccggagcgctagcttcggtggaggcgctggtgggataccgccctgactgtattgaaattctaacctacgggtcttatcgacccgggagacagtgtcaggtgggcagtttgactggggcggtcgcctcctaaagtgtaacggaggcgcccaaaggttccctcagaatggttggaaatcattcgtagagtgcaaaggcataagggagcttgactgcgagacctacaagtcgagcagggacgaaagtcgggcttagtgatccggtggttccgcatggaagggccatcgctcaacggataaaagctaccccggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgtgggcgtaggaaatttgagaggagctgtccttagtacgagaggaccgggatggacgcaccgctggtgtaccagttgttctgccaagggcatagctgggtagctatgtgcggaagggataagtgctgaaagcatctaagcatgaagcccccctcaagatgagatttcccatagcgtaagctagtaagatccctgaaagatgatcaggttgataggttcgaggtggaagcatggtgacatgtggagctgacgaatactaatagatcgaggacttaaccat

SEQ ID NO: 51 - DNA, 2912 bp, Enterococcus faecalis
ggttaagtgaataagggcgcacggtggatgccttggcactaggagccgatgaaggacgggactaacaccgatatgctttggggagctgtaagtaagctatgatccagagatttccgaatgggggaacccaatatcttttataggatattacttttcagtgaatacatagctgattagaggtagacgcagagaactgaaacatcttagtacctgcaggaagagaaagaaaattcgattccctgagtagcggcgagcgaaacgggaagagcccaaaccaacaagcttgcttgttggggttgtaggactccaatatggtagtctgttagtatagttgaaggatttggaaaattccgctaaagagggtgaaagccccgtagacgaaatgctaacaacacctaggaggatcctgagtacggcggaacacgagaaattccgtcggaatccgcggggaccatcccgcaaggctaaatactccctagtgaccgatagtgaaccagtaccgtgagggaaaggtgaaaagcaccccggaaggggagtgaaatagatcctgaaaccgtgtgcctacaacaagtcaaagctcgttaatgagtgatggcgtgccttttgtagaatgaaccggcgagttacgattgcatgcgaggttaagtcgaagagacggagccgcagcgaaagcgagtctgaatagggcgaatgagtatgtagtcgtagacccgaaaccatgtgatctacccatgtccaggttgaaggtgcggtaaaacgcactggaggaccgaacccacgtacgttgaaaagtgcggggatgaggtgtgggtagcggagaaattccaaacgaacttggagatagctggttctctccgaaatagctttagggctagcctcggaattgagaatgatggaggtagagcactgtttggactaggggcccatctcgggttaccgaattcagataaactccgaatgccattcatttatatccgggagtcagactgcgagtgataagatccgtagtcgaaagggaaacagcccagaccaccagctaaggtcccaaaatatatgttaagtggaaaaggatgtggggttgcacagacaactaggatgttggcttagaagcagccaccatttaaagagtgcgtaatagctcactagtcgagtgaccctgcgccgaaaatgtaccggggctaaacatattaccgaagctgtggactacaccattaggtgtagtggtaggagagcgttctaagggcgttgaaggtcgatcgtgaggacggctggagcgcttagaagtgagaatgccggtatgagtagcgaaagacaggtgagaatcctgtccaccgtatgactaaggtttcctggggaaggctcgtccgcccagggttagtcgggacctaagccgaggccgataggcgtaggcgatggacaacaggttgatattcctgtaccagttgtttttgtttgagcaatggagggacgcagtaggctaaggaatgcatgcgattggaagtgcatgtccaagcaatgagtcttgagtagagttaaatgctttactctttaaggacaagttgtgacggggagcgaaataatagtagcgaagttcctgatgtcacactgccaagaaaagcttctagtgagaaaacaactgcccgtaccgtaaaccgacacaggtagtcgaggagagtatcctaaggtgagcgagcgaactctcgttaaggaactcggcaaaatgaccccgtaacttcgggagaaggggtgctgacttcggtcagccgcagtgaataggcccaagcgactgtttatcaaaaacacaggtctctgcaaaatcgtaagatgaagtataggggctgacgcctgcccggtgctggaaggttaagaggatgggttagcttcggcgaagctcagaattgaagccccagtaaacggcggccgtaactataacggtcctaaggtagcgaaattccttgtcgggtaagttccgacccgcacgaaaggcgtaacgatttgggcactgtctcaacgagagactcggtgaaattttagtacctgtgaagatgcaggttacccgcgacaggacggaaagaccccatggagctttactgtagtttgatattgagtgtttgtaccacatgtacaggataggtaggagccgatgagaccggaacgctagtttcggaggaggcgctggtgggatactacccttgtgttatgaaccctctaacccgcaccactaatcgtggtgggagacagtgtcagatgggcagtttgactggggcggtcgcctcctaaaaggtaacggaggcgcccaaaggttccctcagaatggttggaaatcattcgaagagtgtaaaggcagaagggagcttgactgcgagacctacaagtcgagcagggacgaaagtcgggcttagtgatccggtggttccgcatggaagggccatcgctcaacggtaaaagctaccctggggataacaggcttatctcccccaagagtccacatcgacggggaggtttggcacctcgatgtcggctcgtcgcatcctggggctgtagtcggtcccaagggttgggctgttcgcccattaaagcggcacgcgagctgggttcagaacgtcgtgagacagttcggtccctatccgtcgcgggcgttggaaatttgagaggagctgtccttagtacgagaggaccgggatggacttaccgctggtgtaccagttgttctgccaagggcattgctgggtagctatgtagggaagggataaacgctgaaagcatctaagtgtgaagcccacctcaagatgagatttcccatttctttaagaaagtaagacccctgagagatgatcaggtagataggttggaagtggaaggctagtgatagttggagcggaccaatactaatcggtcgaggacttaaccaa

SEQ ID NO: 52 - DNA, 2898 bp, Lactococcus lactis
ggcaaagttaataagggcgcacggtggatgccttggcactaagagccgatgaaggacgtgactaacgacgatattctagggggagcagtaagtacgcattgatccctaggtctccgaatgggaaaacccagctgctactagcagttattcatgagtgaatacatagctcatgtaaaggtaacgcagagaactgaaacatctaagtacctgcaggaagagaaagtaaaaacgatttcgtaagtagcggcgagcgaacgcgaagaagggcaaaccaagaagcttgcttcttggggttgtaggactgcaacgtggacttaagcattatagtcgaataacctgggaaggttaatcaaagagggtaataatcccgtagacgaaatagcgcttatacctagcagtatcctgagtagggctggacacgcgaaatccagtttgaatccgggaggaccatctcccaaccctaaatactccttagtgaccgatagtgaaccagtaccgtgagggaaaggtgaaaagaacccgagaggggagtgaaatagcacctgaaaccgtgtgcctacaagaagttcgagcccgttaatgggtgagagcgtgccttttgtagaatgaaccggcgagttacgttatgatgcgaggttaagttgaagagacggagccgtagggaaaccgagtctgaatagggcgacttagtatcatgatgtagacccgaaacctagtgacctatccatgagcagggtgaaggtgtggtaagacgcactggaggcccgaaccaggacacgttgaaaagtgtttggatgacttgtggatagcggagaaattccaaacgaactgggagatagctggttctctccgaaatagctttagggctagcgtcgaaatgtaagtgtattggaggtagagcactgtttgggtgaggggtccgtctaggattaccaatctcagataaactccgaatgctaatacacatgttcggcagtcagactgcgagtgctaagatccgtagtcgaaagggaaacagcccagaccaacagctaaggtcccaaaatatatgttaagtggaaaaggatgtggggttgcacagacaactaggatgttagctcagaagcagctatcattcaaagagtgcgtaatagctcactagtcgagtgaccctgcgccgaaaatgtaccggggctaaacatattaccgaagctttggattgatattttatcaatggtaggagagcgttcttaaccgcgatgaaggtataccgtgaggagtgctggagcgttaagaagtgagaatgccggtatgagtagcgcaagataagtgagaatcttatccaccgtaagactaaggtttccaggggaaggctcgtccgccctgggttagtcgggacctaaggcgaggccgaaaggcgtagtcgatggacaactggttgatattccagtactagatatgatcgtgatggagggacgcagtaggctaagagatgccagttaatggattctggtctaagcagtgaggtgtgagatgtgtcaaatgcatttctctttaacattgagctgtgatggggaagcaactacggttgcgaactctctgatgtcacactgccaagaaaagcttctagcgtaaagtcatatctacccgtaccgcaaaccgacacaggtggtcgaggcgagtagcctcaggtgatcgagagaactctcgttaaggaactcggcaaaatagccccgtaacttcgggagaaggggtgctggtgtaaaagccagccgcagtgaataggcccaagcaactgtttatcaaaaacacagctctctgctaaaccgcaaggtgatgtatagggggtgacgcctgcccggtgctggaaggttaagaggagtgcttagacgtaagtcgaaggtatgaattgaagccccagtaaacggcggccgtaactataacggtcctaaggtagcgaaattccttgtcgggtaagttccgacccgcacgaaaggcgtaatgatttgggcactgtctcaacgagagactcggtgaaattttagtacctgtgaagatgcaggttacccgcgacaggacggaaagaccccatggagctttactgtagtttgatattgagtacctgtaagtcatgtacaggataggtaggagccattgaaatagggacgctagtttctattgaggcgttgttgggatactacccttgacttatggttactctaacccgctggcataatcggccagggagacagtgtctgacggacagtttgactggggcggtcgctcctaaagagtaacggaggcgctcaaaggttggctcagattggttggaaatcaatcgtagagtgtaaaggtaaaagccagcttgactgcgagagctacaactcgagcaggtaggaaactaggacttagtgatccggtggtaccgcatggaagggccatcgctcaacggataaaagctaccctggggataacaggcttatctcccccaagagttcacatcgacggggaggtttggcacctcgatgtcggctcgtcgcatcctggggctgtagtcggtcccaagggttgggctgttcgccattaaagcggcacgcgagctgggttcagaacgtcgtgagacagttcggtccctatccgtcgcgggcgtaggtaatttgagaggatctgtccttagtacgagaggaccgggatggacttaccgctggtgtaccagttgttccgccaggagcacggctggatagctatgtagggaagggataagcgctgaaagcatctaagtgcgaagcccacctcaagatgagattacccattcgtaagaattaagagcccagagagatgatctggtagataggctggaagtggaagagttgcgagacttggagcggaccagtactaatcgctcgaggactttaccaa

SEQ ID NO: 53 - DNA, 2932 bp, Listeria monocytogenes
ggttaagttagaaagggcgcacggtggatgccttggcactaggagccgaagaaggacgggactaacaccgatatgctttggggagctgtacgtaagcgttgatccagagatttccgaatgggggaacccactatctttagtcggatagtatccttacgtgaatacatagcgtgaggaaggcagacccagggaactgaaacatctaagtacctggaggaagagaaagaaaaatcgatttcctgagtagcggcgagcgaaacggaaagagcccaaaccaagaagcttgcttcttggggttgtaggacactctatacggagttacaaaagaaagttataaatgaagcggtctggaaaggcccgccaaagacggtaacagcccggtagttgaaatggctttccctccagagtggatcctgagtacggcggaacacgtgaaattccgtcggaatccgggaggaccatctcccaaggctaaatactccctagtgaccgatagtgaaccagtaccgtgagggaaaggtgaaaagcaccccggaaggggagtgaaacagttcctgaaaccgtgtgcctacaagtagttagagcccgttaatgggtgatagcgtgccttttgtagaatgaaccggcgagttacgatttgttgcaaggttaagcggaaaaagcggagccgtagcgaaagcgagtctgaatagggcgcataagtaacaggtcgtagacccgaaaccaggtgatctacccatgtccaggatgaaggtaaggtaatacttactggaggtccgaacccacgcacgttgaaaagtgcggggatgaggtgtgggtagcggagaaattccaatcgaacttggagatagctggttctctccgaaatagctttagggctagcctcgaggtaaagagtcatggaggtagagcactgtttggactaggggcccttctcgggttaccgaattcagataaactccgaatgccatgtacttatactcgggagtcagactgcgagtgataagatccgtagtcgaaagggaaacagcccagaccaccagttaaggtccccaaatatatgttaagtggaaaaggatgtggggttgcttagacaaccaggatgttggcttagaagcagccaccattgaaagagtgcgtaatagctcactggtcgagtgaccccgcgccgaaaatgtaccggggctaaacatattaccgaaactgtggatgaacctctttagaggttcgtggtaggagagcgttctaagggcggtgaagtcagaccggaaggactggtggagcgcttagaagtgagaatgccggtatgagtagcgaaagaagggtgagaatcccttccaccgaatatctaaggtttcctgaggaaggctcgtccgctcagggttagtcgggacctaagccgaggccgataggcgtaggcgatggacaacaggtagagattcctgtaccagtgctaattgtttaaccgatggggtgacacagaaggatagggaatcgcacgaatggaaatgtgcgtccaagcagtgagtgtgagaagtaggcaaatccgcttctcacgaagcatgagctgtgatggggaaggaaattaagtacggaagttcctgatttcacgctgtcaagaaaagcctctaggaagagtagtactgcccgtaccgcaaaccgacacaggtagatgaggagagaatcctaaggtgagcgagagaactctcgttaaggaactcggcaaaatgaccccgtaacttcgggagaaggggtgctctattagggtgcaagcccgagagagccgcagtgaataggcccaggcgactgtttagcaaaaacacaggtctctgcaaaaccgtaaggtgacgtataggggctgacgcctgcccggtgctggaaggttaagaggagtgcttagcttcggcgaaggtacgaattgaagccccagtaaacggcggccgtaactataacggtcctaaggtagcgaaattccttgtcgggtaagttccgacccgcacgaaaggcgcaacgatctgggcactgtctcaacgagagactcggtgaaattatagtacctgtgaagatgcaggttacccgcgacaggacggaaagaccccgtggagctttactgcaacctgatatggaatgtttgtaccgcttgtacaggataggtaggagccgaagagacgtgtgcgctagcatacgaggaggcaatggtgggatactaccctggctgtatgaccattctaacccgccacgcttagcgcgtggggagacagtgtcaggtgggcagtttgactggggcggtcgcctcctaaagagtaacggaggcgcccaaaggttccctcagaatggatggaaatcattcgcagagtgtaaaggcacaagggagcttgactgcgagactgacaagtcgagcagggacgaaagtcgggcttagtgatccggtggttccgcatggaagggccatcgctcaacggataaaagctaccccggggataacaggcttatctcccccaagagtccacatcgacggggaggtttggcacctcgatgtcggctcgtcgcatcctggggctgtagtcggtcccaagggttgggctgttcgcccattaaagcggcacgcgagctgggttcagaacgtcgtgagacagttcggtccctatccgtcgcgggcgcaggaaatttgagaggagctgtccttagtacgagaggaccgggatggacacaccgctggtgtaccagttgttccgccaggagcatcgctgggtagctatgtgtggcagggataaacgctgaaagcatctaagcgtgaagcccccctcaagatgagatttcccatttcttcggaaagtaagatccctgaaagatgatcaggtagataggtttggagtggaagtgtagcgatacatggagcggacaaatactaatcgatcgaggacttaaccaa

SEQ ID NO: 54 - DNA, 2923 bp, Staphylococcus aureus
gattaagttattaagggcgcacggtggatgccttggcactagaagccgatgaaggacgttactaacgacgatatgctttggggagctgtaagtaagctttgatccagagatttccgaatggggaaacccagcatgagttatgtcatgttatcgatatgtgaatacatagcatatcagaaggcacacccggagaactgaaacatcttagtacccggaggaagagaaagaaaattcgattcccttagtagcggcgagcgaaacgggaagagcccaaaccaacaagcttgcttgttggggttgtaggacactctatacggagttacaaaggacgacattagacgaatcatctggaaagatgaatcaaagaaggtaataatcctgtagtcgaaaatgttgtctctcttgagtggatcctgagtacgacggagcacgtgaaattccgtcggaatctgggaggaccatctcctaaggctaaatactctctagtgaccgatagtgaaccagtaccgtgagggaaaggtgaaaagcaccccggaaggggagtgaaatagaacctgaaaccgtgtgcttacaagtagtcagagcccgttaatgggtgatggcgtgccttttgtagaatgaaccggcgagttacgatttgatgcaaggttaagcagtaaatgtggagccgtagcgaaagcgagtctgaatagggcgtttagtatttggtcgtagacccgaaaccaggtgatctacccttggtcaggttgaagttcaggtaacactgaatggaggaccgaaccgacttacgttgaaaagtgagcggatgaactgagggtagcggagaaattccaatcgaacctggagatagctggttctctccgaaatagctttagggctagcctcaagtgatgattattggaggtagagcactgtttggacgaggggcccctctcgggttaccgaattcagacaaactccgaatgccaattaatttaacttgggagtcagaacatgggtgataaggtccgtgttcgaaagggaaacagcccagaccaccagctaaggtcccaaaatatatgttaagtggaaaaggatgtggcgttgcccagacaactaggatgttggcttagaagcagccatcatttaaagagtgcgtaatagctcactagtcgagtgacactgcgccgaaaatgtaccggggctaaacatattaccgaagctgtggattgtcctttggacaatggtaggagagcgttctaagggcgttgaagcatgatcgtaaggacatgtggagcgcttagaagtgagaatgccggtgtgagtagcgaaagacgggtgagaatcccgtccaccgattgactaaggtttccagaggaaggctcgtccgctctgggttagtcgggtcctaagctgaggccgacaggcgtaggcgatggataacaggttgatattcctgtaccacctataatcgttttaatcgatggggggacgcagtaggataggcgaagcgtgcgattggattgcacgtctaagcagtaaggctgagtattaggcaaatccggtactcgttaaggctgagctgtgatggggagaagacattgtgtcttcgagtcgttgatttcacactgccgagaaaagcctctagatagaaaataggtgcccgtaccgcaaaccgacacaggtagtcaagatgagaattctaaggtgagcgagcgaactctcgttaaggaactcggcaaaatgaccccgtaacttcgggagaaggggtgctctttagggttaacgcccagaagagccgcagtgaataggcccaagcgactgtttatcaaaaacacaggtctctgctaaaccgtaaggtgatgtataggggctgacgcctgcccggtgctggaaggttaagaggagtggttagcttctgcgaagctacgaatcgaagccccagtaaacggcggccgtaactataacggtcctaaggtagcgaaattccttgtcgggtaagttccgacccgcacgaaaggcgtaacgatttgggcactgtctcaacgagagactcggtgaaatcatagtacctgtgaagatgcaggttacccgcgacaggacggaaagaccccgtggagctttactgtagcctgatattgaaattcggcacagcttgtacaggataggtaggagcctttgaaacgtgagcgctagcttacgtggaggcgctggtgggatactaccctagctgtgttggctttctaacccgcaccacttatcgtggtgggagacagtgtcaggcgggcagtttgactggggcggtcgcctcctaaaaggtaacggaggcgctcaaaggttccctcagaatggttggaaatcattcatagagtgtaaaggcataagggagcttgactgcgagacctacaagtcgagcagggtcgaaagacggacttagtgatccggtggttccgcatggaagggccatcgctcaacggataaaagctaccccggggataacaggcttatctcccccaagagttcacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgtgggcgtaggaaatttgagaggagctgtccttagtacgagaggaccgggatggacatacctctggtgtaccagttgtcgtgccaacggcatagctgggtagctatgtgtggacgggataagtgctgaaagcatctaagcatgaagcccccctcaagatgagatttcccaacttcggttataagatccctcaaagatgatgaggttaataggttcgaggtggaagcatggtgacatgtggagctgacgaatactaatcgatcgaagacttaatcaa

SEQ ID NO: 55 - DNA, 2900 bp, Streptococcus mutans
gttaagttaataagggcgcacggtggatgcctaggcactaggagccgatgaaggacgtgacgaacgacgacatgctttggggagctgtaagtaagccttgatccagagatatccgaatgggggaacccaacaggtaatgcctgttatccataactgttaaggttatgagaaggaagacgcagtgaactgaaacatctcagtagctgcaggaagagaaagcaagagcgattgcctcagtagcggcgagcgaagaggcaggagggcaaaccagagtgtttacactctggggttgtaggactgcgataaagcagccaagggaatagaagaagactctgggaagagtcgccagagagagtaagagcctcgtatttgaaattcacttgatgccaagcaggatcctgagtacggcgggacacgaggaatcccgtcggaatctgggaggcccatctcccaaccctaaatactccctagtgaccgatagtgaaccagtaccgtgagggaaaggtgaaaagtaccccggaaggggagtgaaagagaacctgaaaccgtgtgcttacaagaagttcgagcccgttaatgggtgagagcgtgccttttgtagaatgaaccggcgagttacgtttacgtgcgaggttaagttgaagagacggagccgtagggaaaccgagtctgaaaagggcggttaagtacgtagatgtagacccgaaaccaagtgacctacccatgagcaggttgaaggtgcggtaaaacgcactggaggaccgaaccaggacacgttgaaaagtgtttggatgacttgtgggtagcggagaaattccaaacgaacttggagatagctggttctctccgaaatagctttagggctagcgtcggtcgcgagactcttggaggtagagcactgtttgattgaggggtccatcccggattaccaatctcagataaactccgaatgccaacgagttaagaccggcagtcagactgcgagtgctaagatccgtagtcgaaagggaaacagcccagaccaccagctaaggtccccaaataattgttaagtggaaaaggatgtggggttgcacagacaactaggatgttagcttagaagcagctattcattcaaagagtgcgtaatagctcactagtcgagtgaccctgcgccgaaaatgtaccggggctgaaacaatttaccgaagctgtggatcccttaggggatggtaggagagcgttctatgtgcgcagaaggtgtaccgcaaggagcgctggagtgcatagaagtgagaatgccggtatgagtagcgtaagacaggtgagaatcctgtccaccgtaagactaaggattccaggggaaggctcgtccgccctgggttagtcgggacctaaggagagaccgataggtgtatccgatgggcaacaggttgatattcctgtactagagtattgagtgaaggagggacgcagcaggctaactagagcgtgcgattggaagagcacgtccaagcagtgaggtgaggactgagtcaaatgcttagttctgcgccaccaagctgtgacggggagcgaagtttagtagcgaagctagtgatgtcactctgccaagaaaagcttctagcgttaatgaatactctacccgtaccgcaaaccgacacaggtagtcgaggcgagtagcctcaggtgatcgagcgaactctcgttaaggaactcggcaaaatggccccgtaacttcgggagaaggggcgctggcgataagtcagccgcagtgaaaaggcccaagcaactgtttatcaaaaacacagctctctgcgaaatcgtaagatgaagtatagggggtgacgcctgcccggtgctggaaggttaagaggagcgcttagacgtttgtcgaaggtgtgaattgaagccccagtaaacggcggccgtaactataacggtcctaaggtagcgaaattccttgtcgggtaagttccgacccgcacgaaaggcgtaatgatttgggcactgtctcaacgagagactcggtgaaattttagtacctgtgaagatgcaggttacccgcgacaggacggaaagaccccatggagctttactgcagtttgatattgcgtatctgttacacatgtacaggataggtaggagccaaggaagagtgaacgctagtttacttggaggcgttgttgggatactacccttgtgtgatggctactctaacccggtaggttgatcatctacggagacagtgtctgacgggcagtttgactggggcggtcgcctcctaaagcgtaacggaggcgcccaaaggttccctcagactggttggaaatcagtcgtagagtgtaaaggtataagggagcttgactgcgagacagacaagtcgagcagggacgaaagtcgggcttagtgatccggtggtaccgtatggaagggccatcgctcaacggataaaagctaccctggggataacaggcttatctcccccaagagttcacatcgacggggaggtttggcacctcgatgtcggctcgtcgcatcctggggctgtagtcggtcccaagggttgggctgttcgcccattaaagcggcacgcgagctgggttcagaacgtcgtgagacagttcggtccctatccgtcgcgggcgaaggaaatttgagaggatctgctcctagtacgagaggaccagagtggacttaccgctggtgtaccagttgttctgccaagagcatcgctgggtagctaagtagggaggggataaacgctgaaagcatctaagtgtgaagcccccctcaagatgagatttcccataacgttcagttagtaagagccctgaaagaagaacaggtagataggttgggagtggaagcgttgtgagacgtgaagcggaccaatactaatcgctcgaggacttatccaa

SEQ ID NO: 56 - DNA, 2902 bp, Streptococcus pneumoniae
ggttaagttaataagggcgcacggtggatgccttggcactaggagccgacgaaggacgtgacaaacgacgatatgccttgggtagctgtaagtaagcgatgatccagggatttccgaatgggggaacccaacaggtaatacctgttacccacatctgttaaggatgtgaggaggaagacgcagtgaactgaaacatctaagtagctgcaggaagagaaagcaaaagcgattgccttagtagcggcgagcgaaacggcagaagggcaaaccgaagagtttactcttcggggttgtaggactgcaatgtggactcaaagattatagaagaatgatttgggaagatcagccaaagagagtaatagcctcgtatttaaaatagtctttgtacttagcagtatcctgagtacggcgggacacgtgaaatcccgtcggaatctgggaggaccatctcccaaccctaaatactccctagtgaccgatagtgaaccagtaccgtgagggaaaggtgaaaagcaccccgggaggggagtgaaatagaacctgaaaccgtgtgcctacaacaagttcgagcccgttaatgggtgagagcgtgccttttgtagaatgaaccggcgagttacgttatgatgcgaggttaagttgaagagacggagccgtagggaaaccgagtctgaatagggcgccttagtatcatgacgtagacccgaaaccatgtgacctacccatgagcaggttgaaggtgcggtaagacgcactggaggaccgaaccagggcacgttgaaaagtgcttggatgacttgtgggtagcggagaaattccaaacgaacttggagatagctggttctctccgaaatagctttagggctagcgtcgacattagagattcttggaggtagagcactgtttgggtgaggggtccatcccggattaccaatctcagataaactccgaatgccaatgaattatggtcggcagtcagactgcgagtgctaagatccgtagtcgaaagggaaacagcccagaccaccagctaaggtcccaaaataattgttaagtggaaaaggatgtggggttgcacagacaactaggatgttagcttagaagcagctattcattcaaagagtgcgtaatagctcactagtcgagtgaccctgcgccgaaaatgtaccggggctaaaacaatttaccgaagctgtggatacctttataggtatggtaggagagcgttctatgtgtgatgaaggtataccgtgaggagtgctggaacgcatagaagtgagaatgccggtatgagtagcgaaagacaggtgagaatcctgtccaccgtaagactaaggtttccaggggaaggctcgtccgccctgggttagtcgggacctaaggagagaccgaaaggtgtatccgatggacaacaggttgatattcctgtactagagtatgtagtgatggagggacgcagtaggctaactaaagcagacgattggaagagtctgtctaagcagtgaggtgtgaattgagtcaaatgcttaattctataacattgagctgtgatggggagcgaagtttagtagcgaagttagtgacgtcacactgccaagaaaagcttctagcgtttaaacatactctacccgtaccgcaaaccgacacaggtagtcgaggcgagtagcctcaggtgagcgagagaactctcgttaaggaactcggcaaaatgaccccgtaacttcgggagaaggggtgctgacttaaagtcagccgcagtgaataggcccaagcaactgtttatcaaaaacacagctctctgctaaatcgtaagatgatgtatagggggtgacgcctgcccggtgctggaaggttaagaggagtgcttagcgtaagcgaaggtatgaattgaagccccagtaaacggcggccgtaactataacggtcctaaggtagcgaaattccttgtcgggtaagttccgacccgcacgaaaggcgtaatgatttgggcactgtctcaacgagagactcggtgaaattttagtacctgtgaagatgcaggttacccgcgacaggacggaaagaccccatggagctttactgcagtttgatattgagtgtctgtaccacatgtacaggataggtaggagtctaagagatcgggacgccagtttcgaaggagacgctgttgggatactacccttgtgttatggccactctaacccagataggtgatccctatcggagacagtgtctgacgggcagtttgactggggcggtcgcctcctaaaaggtaacggaggcgcccaaaggttccctcagaatggttggaaatcattcgcagagtgtaaaggtataagggagcttgactgcgagagctacaactcgagcagggacgaaagtcgggcttagtgatccggtggttccgtatggaagggccatcgctcaacggataaaagctaccctggggataacaggcttatctcccccaagagttcacatcgacggggaggtttggcacctcgatgtcggctcgtcgcatcctggggctgtagtcggtcccaagggttgggctgttcgcccattaaagcggcacgcgagctgggttcagaacgtcgtgagacagttcggtccctatccgtcgcgggcgtaggaaatttgagaggatctgctcctagtacgagaggaccagagtggacttaccgctggtgtaccagttgtcttgccaaaggcatcgctgggtagctatgtagggaagggataaacgctgaaagcatctaagtgtgaaacccacctcaagatgagatttcccatgattatatatcagtaagagccctgagagatgatcaggtagataggttagaagtggaagtgtggcgacacatgtagcggactaatactaatagctcgaggacttatccaa

SEQ ID NO: 57 - DNA, 2901 bp, Streptococcus pyogenes
ggttaagttaataagggcgcacggtggatgccttggcactagaagccgaagaaggacgtgactaacgacgaaatgctttggggagctgtaagtaagcgctgatccagagatgtccgaatgggggaacccggcatgtaatgcatgtcatccatgactgttaaggtcatgagaaggaagacgcagtgaactgaaacatctaagtagctgcaggaagagaaagcaaacgcgattgccttagtagcggcgagcgaaacggcaggagggcaaaccgaggagtttactcctcggggttgtaggactgcgaagtgggacataaagttaatagaagaattacctgggaaggtaagccaaagagagtaacagcctcgtatttaaaattgactttagccctagcagtatcctgagtacggcgagacacgcgaaatctcgtcggaatctgggaggaccatctcccaaccctaaatactctctagtgaccgatagtgaaccagtaccgtgagggaaaggtgaaaagcaccccgggaggggagtgaaatagaacctgaaaccgtgtgcctacaacaagttcgagcccgttaatgggtgagagcgtgccttttgtagaatgaaccggcgagttacgatatgatgcgaggttaagttgaagagacggagccgtagggaaaccgagtcttaatagggcgtcatagtatcatgttgtagacccgaaaccatgtgacctacccatgagcaggttgaaggtgtggtaaaacgcactggaggaccgaaccagggcacgttgaaaagtgcttggatgacttgtgggtagcggagaaattccaaacgaacttggagatagctggttctctccgaaatagctttagggctagcgtcgatgttaagtctcttggaggtagagcactgtttgggtgaggggtccatcccggattaccaatctcagataaactccgaatgccaacgagatataatcggcagtcagactgcgagtgctaagatccgtagtcgaaagggaaacagcccagaccaccagctaaggtcccaaaataactgttaagtggaaaaggatgtggggttgcacagacaactaggatgttagcttagaagcagctattcattcaaagagtgcgtaatagctcactagtcgagtgaccctgcgccgaaaatgtaccggggctaaaacagtttaccgaagctgtggatgacacaaaagtgtcatggtaggagagcgttctatgtgtgaagaaggtgtaccgtgaggagcgctggaacgcatagaagtgagaatgccggtatgagtagcgaaagacaggtgagaatcctgtccaccgtaagactaaggtttccaggggaaggctcgtccgccctgggttagtcgggacctaaggagagaccgaaaggtgtatccgatggccaacaggttgatattcctgtactagagtatatagtgatggagggacgcagtaggctaactaaaccggacgattggaagagtccggctaagcagtgaggtgtaagatgagtcaaatgcttatctttataacattgagctgtgatggggagcgaattttagtagcgaagttagtgatgtcacactgccaagaaaagcttctagcgtttaatgatactctacccgtaccgcaaaccgacacaggtagtcgaggcgagtagcctcaggtgatcgagagaactctcgttaaggaactcggcaaaatgaccccgtaacttcgggagaaggggtgctgacttaggtcagccgcagtgaataggcccaagcaactgtttatcaaaaacacagctctctgctaaatcgtaagatgatgtatagggggtgacgcctgcccggtgctggaaggttaagaggagggtttagcgcaagcgaagatctgaattgaagccccagtaaacggcggccgtaactataacggtcctaaggtagcgaaattccttgtcgggtaagttccgacccgcacgaaaggcgtaatgatttgggcactgtctcaacgagagactcggtgaaattttagtacctgtgaagatgcaggttacccgcgacaggacggaaagaccccatggagctttactgcagtttgatattgagtatctgtaccacatgtacaggataggtaggagccattgacttcgggacgccagtttcgaatgaggcgttgttgggatactacccttgtgttatggctactctaacccagataggttatccctatcggagacagtgtctgacgggcagtttgactggggcggtcgcctcctaaagagtaacggaggcgcccaaaggttccctcagattggttggaaatcaatcgcagagtgtaaaggtataagggagcttgactgcgagagctacaactcgagcagggacgaaagtcgggcttagtgatccggtggtaccgaatggaagggccatcgctcaacggataaaagctaccctggggataacaggcttatctcccccaagagttcacatcgacggggaggtttggcacctcgatgtcggctcgtcgcatcctggggctgtagtcggtcccaagggttgggctgttcgcccattaaagcggcacgcgagctgggttcagaacgtcgtgagacagttcggtccctatccgtcgcgggcgtaggaaatttgagaggatctgctcctagtacgagaggaccagagtggacttaccgctggtgtaccagttgtcttgccaaaggcatcgctgggtagctatgtagggaagggataagcgctgaaagcatctaagtgcgaagcccccctcaagatgagatttcccatgattttatatcagtaagagccctgagagatgatcaggtagataggttaggagtgtaagtgtagcgatacatgtagcggactaatactaatagctcgaggacttatcc

SEQ ID NO: 58 - DNA, 3107 bp, Mycobacterium avium
tgtgtgtaagtaagtgtttaagggcgcatggtggatgccttggcatcgagagccgatgaaggacgtgggaggctgcgatatgcctcggggagctgtcaaccgagcattgatccgaggatttccgaatgggggaacccagcacgagtgatgtcgtgttacccgtatctgaatatatagggtgcgggaggtaacgcggggaagtgaaacatctcagtacccgtaggagaagaaaacaattgtgattccgtcagtagtggcgagcgaaccggaacaggctaaaccgcatgcatggacaaccgggtaggggttgtgtgtgcggggttgtgggattgatatgtctcagctctacctggctgaggggtagtcagaaagtgtcgtggttagcggaagtggcctgggacggcccgccgtagacggtgagagcccggtacgcgaaaacccggcacctgccttatatcaacacccgagtagcagcgggcccgtggaatctgctgtgaatctgccgggaccacccggtaagcctaaatacttctcgatgaccgatagcggattagtaccgtgagggaatggtgaaaagtaccccgggagggagtgaaatagtacctgaaaccgtgtgcctacaatccgtcagagcctcctcgtggggtgatggcgtgccttttgaagaatgagcctgcgagtcagggacacgtcgcgaggttaacccgtgcggggtagccgcagcgaaagcgagtctgaatagggcgcatcccctttggggtgtagtggcgtgttctggacccgaagcggagtgatctacccatggccagggtgaagcgcgggtaagaccgcgtggaggcccgaacccacttaggttgaagactgaggggatgagctgtgggtaggggtgaaaggccaatcaaactccgtgatagctggttctccccgaaatgcatttaggtgcagcgttgcgtggttcaccacggaggtagagctactggatggccgatgggccctactaggttactgacgtcagccaaactccgaatgccgtggtgtaaaagcgtggcagtgagacggcgggggataagctccgtacgtcgaaagggaaacagcccagatcgccggctaaggcccctaagcgtgtgctaagtggaaaaggatgtgtagtcgcagagacaaccaggaggttggcttagaagcagccatccttgaaagagtgcgtaatagctcactggtcaagtgattatgcgccgataatgtagcggggctcaagcacaccgccgaagccgcggcacattcatctttacggtggatgtgggtaggggagcgtcccccattcagcgaagctccgggtgaccggtggtggagggtgggggagtgagaatgcaggcatgagtagcgataaggcaagtgagaaccttgcccgccgtaagaccaagggttcctgggccaggccagtccgcccagggtgagtcgggacctaaggcgaggccgacagggtagtcgatggacaacgggttgatattcccgtacccgtgtatgggcgtccctgatgaatcagcggtactaaccacccaaaaccggatcgaccattccccttcgggggcgtggcgattcggggctgcgtgggaccttcgctggtagtagtcaagcaatggggtgacgcaggaaggcagccgtaccagtcagtggtaatactggggcaagcccgtagagagcgataggcaaatccgtcgctcactaatcctgagaggtgatgcatagccggttgaggcgaattcggtgatcctctgctgccaagaaaagcctctagcgagcacatacacggcccgtaccccaaaccaacacaggtggtcaggtagagaataccaaggcgtacgagataactatggttaaggaactcggcaaaatgcccccgtaacttcgggagaagggggcccggaataccgtgaacacccttgcggtgggagcgggattcggccgcagaaaccagtgggtagcgactgtttactaaaaacacaggtccgtgcgaagtcgcaagacgatgtatacggactgacgcctgcccggtgctggaaggttaagaggacccgttaacccgtaagggtgaagcggagaatttaagccccagtaaacggcggtggtaactataaccatcctaaggtagcgaaattccttgtcgggtaagttccgacctgcacgaatggcgtaacgacttcccaactgtctcaaccatagactcggcgaaattgcactacgagtaaagatgctcgttacgcgcggcaggacgaaaagaccccgggaccttcactacaacttggtattggtgttcggtacggtttgtgtaggataggtgggagactttgaagcacagacgccagtttgtgtggagtcgttgttgaaataccactctgatcgtattggacacctaacgtcgaacccttatcgggttcacggacagtgcctggcgggtagtttaactggggcggttgcctcctaaaatgtaacggaggcgcccaaaggttccctcaacctggacggcaatcaggtggcgagtgtaagtgcacaagggagcttgactgcgagacttacaagtcaagcagggacgaaagtcgggactagtgatccggcacccccgagtggaaggggtgtcactcaacggataaaaggtaccccggggataacgggctgatcttccccaagagtccatatcgacgggatggtttggcacctcgatgtcggctcgtcgcatcctggggctggagcaggtcccaaaggttgggctgttcgcccattaaagcggcacgcgagctgggtttagaacgtcgtgagacagttcggtctctatccgccgcgcgcgtcagaaacttgaggaaacctgtccctagtacgagaggaccgggacggacgaacctctggtataccagttgtcccaccaggggcacggctggatagccacgttcggacaggataaccgctgaaagcatctaagcgggaaaccttctccaagatcaggtttctcacccttttagagggataaggccccccgcagaccacgggattgataggccagacctggaagctcagtaatgagtgcagggaactggcactaactggccgaaagc

SEQ ID NO: 59 - DNA, 3138 bp, Mycobacterium tuberculosis
ttgtaagtgtctaagggcgcatggtggatgccttggcatcgagagccgatgaaggacgtgggaggctgcgatatgcctcggggagctgtcaaccgagcgtggatccgaggatttccgaatggggaaacccagcacgagtgatgtcgtgctacccgcatctgaatatatagggtgcgggagggaacgcggggaagtgaaacatctcagtacccgtaggaggagaaaacaattgtgattccgcaagtagtggcgagcgaacgcggaacaggctaaaccgcacgcatgggtaaccgggtaggggttgtgtgtgcggggttgtgggaggatatgtctcagcgctacccggctgagaggcagtcagaaagtgtcgtggttagcggaagtggcctgggatggtctgccgtagacggtgagagcccggtacgcgaaaacccggcacctgcctagtatcaattcccgagtagcagcgggcccgtggaatccgctgtgaatccgccgggaccacccggtaagcctaaatactcctcgatgaccgatagcggattagtaccgtgagggaatggtgaaaagtaccccgggaggggagtgaaagagtacctgaaaccgtgtgcctacaatccgtcagagcctccttttcctctccggaggagggtggtgatggcgtgccttttgaagaatgagcctgcgagtcagggacatgtcgcaaggttaacccgtgtggggtagccgcagcgaaagcgagtctgaatagggcgacccacacgcgcatacgcgcgtgtgaatagtggcgtgttctggacccgaagcggagtgatctacccatggccagggtgaagcgcgggtaagaccgcgtggaggcccgaacccacttaggttgaagactgaggggatgagctgtgggtaggggtgaaaggccaatcaaactccgtgatagctggttctccccgaaatgcatttaggtgcagcgttgcgtggttcaccgcggaggtagagctactggatggccgatgggccctactaggttactgacgtcagccaaactccgaatgccgtggtgtaaagcgtggcagtgagacggcgggggataagctccgtacgtcgaaagggaaacagcccagatcgccggctaaggcccccaagcgtgtgctaagtgggaaaggatgtgcagtcgcaaagacaaccaggaggttggcttagaagcagccacccttgaaagagtgcgtaatagctcactggtcaagtgattgtgcgccgataatgtagcggggctcaagcacaccgccgaagccgcggcacatccaccttgtggtgggtgtgggtaggggagcgtccctcattcagcgaagccaccgggtgaccggtggtggagggtgggggagtgagaatgcaggcatgagtagcgacaaggcaagtgagaaccttgcccgccgaaagaccaagggttcctgggccaggccagtccgcccagggtgagtcgggacctaaggcgaggccgacaggcgtagtcgatggacaacgggttgatattcccgtacccgtgtgtgggcgcccgtgacgaatcagcggtactaaccacccaaaaccggatcgatcactccccttcgggggtgtggagttctggggctgcgtgggaacttcgctggtagtagtcaagcgaaggggtgacgcaggaaggtagccgtaccagtcagtggtaacactggggcaagccggtagggagagcgataggcaaatccgtcgctcactaatcctgagaggtgacgcatagccggttgaggcgaattcggtgatcctctgctgccaagaaaagcctctagcgagcacacacacggcccgtaccccaaaccgacacaggtggtcaggtagagcataccaaggcgtacgagataactatggttaaggaactcggcaaaatgcccccgtaacttcgggagaagggggaccggaatatcgtgaacacccttgcggtgggagcgggatccggtcgcagaaaccagtgaggagcgactgtttactaaaaacacaggtccgtgcgaagtcgcaagacgatgtatacggactgacgcctgcccggtgctggaaggttaagaggacccgttaacccgcaagggtgaagcggagaatttaagccccagtaaacggcggtggtaactataaccatcctaaggtagcgaaattccttgtcgggtaagttccgacctgcacgaatggcgtaacgacttctcaactgtctcaaccatagactcggcgaaattgcactacgagtaaagatgctcgttacgcgcggcaggacgaaaagaccccgggaccttcactacaacttggtattgatgttcggtacggtttgtgtaggataggtgggagactgtgaaacctcgacgccagttggggcggagtcgttgttgaaataccactctgatcgtattgggcatctaacctcgaaccctgaatcgggtttagggacagtgcctggcgggtagtttaactggggcggttgcctcctaaaatgtaacggaggcgcccaaaggttccctcaacctggacggcaatcaggtggcgagtgtaaatgcacaagggagcttgactgcgagacttacaagtcaagcagggacgaaagtcgggattagtgatccggcacccccgagtggaaggggtgtcgctcaacggataaaaggtaccccggggataacaggctgatcttccccaagagtccatatcgacgggatggtttggcacctcgatgtcggctcgtcgcatcctggggctggagcaggtcccaagggttgggctgttcgcccattaaagcggcacgcgagctgggtttagaacgtcgtgagacagttcggtctctatccgccgcgcgcgtcagaaacttgaggaaacctgtccctagtacgagaggaccgggacggacgaacctctggtgcaccagttgtcccgccaggggcaccgctggatagccacgttcggtcaggataaccgctgaaagcatctaagcgggaaaccttctccaagatcaggtttctcacccacttggtgggataaggccccccgcagaacacgggttcaataggtcagacctggaagctcagtaatgggtgtagggaactggtgctaaccggccgaaaacttacaaca

SEQ ID NO: 60 - DNA, 2903 bp, Escherichia coli
ggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgattcgtcacactatcattaactgaatccataggttaatgaggcgaaccgggggaactgaaacatctaagtaccccgaggaaaagaaatcaaccgagattcccccagtagcggcgagcgaacggggaggagcccagagcctgaatcagtgtgtgtgttagtggaagcgtctggaaaggcgcgcgatacagggtgacagccccgtacacaaaaatgcacatactgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcctcttttatggggtgactgcgtaccttttgtataatgggtcagcgacttatattctgtagcaaggttaaccgaataggggagccgaagggaaaccgagtcttaaccgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacatacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacactgtgtgttgttgggtaggggagcgttctgtaagcctgtgaaggtgtactgtgaggtatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaagtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggtcagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgtggacccgtgatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaacctt

SEQ ID NO: 61 - DNA, 2903 bp, Klebsiella pneumoniae
ggttaagcgactaagcgtacacggtggatgccctggcagtcagaggcgatgaaggacgtgctaatctgcgaaaagcgtcggtaaggtgatatgaaccgttataaccggcgatgtccgaatggggaaacccagtgcaattcgttgcactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaacggtctggaaagtccgacggtacagggtgatagtcccgtacaccaaaatgcacaggctgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcaccttcgggtgtgactgcgtaccttttgtataatgggtcagcgacttatattctgtagcaaggttaaccgtataggggagccgcagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaactcatcttcgggggtagagcactgtttcggctagggggtcatcccgacttaccaacccgatgcaaactacgaataccgaagaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttagaagcagccatcatttaaagaaagcgtaatagctcactggtcgagtcggcctgcgcggaagatgtaacggggctaaaccatgcaccgaagctgcggcagcgacactatgtgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttagccgggcgacggttgtcccggtttaagcatgtaggctggttgtccaggcaaatccggataatcaaggctgaggtgtgatgacgaggcactacggtgctgaagtaacaaatgctctgcttccaggaaaagcctctaagcatcaggtaacatcaaatcgtaccccaaaccgacacaggtggtcaggtagagaataccaaggcgcttgagataactcgggtgaaggaactaggcaaaatggtgccgtaacttcgggagaaggcacgctggtgtgtaggtgaagcccctgccgggtggagctgagaccagtcgaagataccagctggctgcaactgtttattaaaaacacagcactgtgcaaacacgaaagtggacgtatacggtgtgacgcctgcccggtgccggaaggttaattgatggggttatccgtaaggagaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagcgtggacgccagtctgcgtggagccaaccttgaaataccaccctttaatgtttgatgttctaacgttggcccctcaccggggttgcggacagtgtctggtgggtagtttgactggggcggtctcctcccaaagcgtaacggaggagcacgaaggttagctaatcctggtcggacatcaggaggttagtgcaatggcataagctagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaattgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgagactttaagtctcctgaaggaacgttgaagacgacgacgttgataggccgggtgtgtaagcgcagcgatgcgttgagctaaccggtactaatgaaccgtgaggcttaacctt

SEQ ID NO: 62 - DNA, 2897 bp, Haemophilus influenzae
gtatagttaagtgactaagcgtacaaggtggatgccttggcaatcagaggcgaagaaggacgtgctaatctgcgaaaagcttggatgagtcgataagaggcgtttaatccaagatatccgaatggggaaacccagtagatgaagaatctactatcaacaagtgaattcatagcttgttgaggcaaaccgggagaactgaaacatctaagtaccccgaggaaaagaaatcaaccgagatttcgtcagtagcggcgagcgaaagcgaaagagccagtaagtgatagcaatatagtgaggagaatgtgttgggaagcacaatcaaagagggtgataatcccgtatctaaaaaccatattgtggtactaagctaacgagaagtagggcgggacacgtgatatcctgtttgaagaaggggggcccatcctccaaggctaaatactcctgattgaccgatagtgaaccagtactgtgaaggaaaggcgaaaagaaccccggtgaggggagtgaaatagaacctgaaaccttgtacgtacaagcagtgggagcgagggcaaccttgtgactgcgtaccttttgtataatgggtcagcgacttatattttgtagcgaggttaaccgaataggggagccgaagggaaaccgagtcttaactgggcgaatagttgcaaggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaatctatttaggtagagccttgaggtgacacctttgggggtagagcactgtttcggctagggggccatcccggcttaccaacccgatgcaaactacgaataccaaagagtgatactcaggagacacacggcgggtgctaacgtccgtcgtggagagggaaacaacccagaccgccagctaaggtccccaagtctatattaagtgggaaacgaagtgggaaggcttagacagctaggatgttggcttagaagcagccatcatttaaagaaagcgtaatagctcactagtcgagtcggcctgcgcggaagatgtaacggggctgaaatatagcaccgaagctgcggcatcagaatttattctgttgggtaggggagcgttgtgtaagcggaagaaggttcatcgagaggtgggctggacgtatcacaagtgcgaatgctgacataagtaacgataaaacgggtgaaaaacccgttcgccggaagaccaagggttcctgtccaacgttaatcggggcagggtgagtcggctcctaaggcgaggctgaaaagcgtagtcgatgggaaacaggttaatattcctgtacttggtaaagctgcgatgtggggacggagtaggttaggttatcgcactgttggatatgtgcgtttaagttggtaggtgggaagtttaggcaaatccggacttccttaacacagagagatgatgacgaggctctacggagctgaagtaactgataccacacttccaggaaaagccactaagcgaaaggctttactaaaccgtactgaaaaccgacacaggtggtcaggtagagaatactcaggcgcttgagagaactcgggtgaaggaactaggcaaaatagcaccgtaacttcgggagaaggtgcgccggcgtagattgtaagggctagcccctgaaggttgaaccggtcgaagataccagctggctgcaactgtttattaaaaacacagcactctgcaaacacgaaagtggacgtatagggtgtgatgcctgcccggtgctggaaggttaattgatggtgtcatcgaaagagaagcacctgatcgaagccccagtaaacggcggccgtaactataacggtcctaaggtagcgaaattccttgtcgggtaagttccgacctgcacgaatggcataatgatggccaggctgtctccacccgagactcagtgaaattgaaatcgccgtgaagatgcggtgtacccgcggctagacggaaagaccccgtgaacctttactatagcttgacactgaacattgaattttgatgtgtaggataggtgggagcctttgaagcagtgacgccagtcattgtggaggcgaccttgaaataccaccctttaacgtttgatgttctaacgaagatgacgaaacgtggtctcggacagtgtctggtgggtagtttgactggggcggtctcctcccaaagcgtaacggaggagcacgaaggtttgctaatcacggtcggacatcgtgaggttagtgcaatggtataagcaagcttaactgcgagacagacaagtcgagcaggtacgaaagtaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgtaggatgattgattggggctgctcctagtacgagaggaccggagtggacgcatcactggtgttccggttgtgtcgccagacgcattgccgggtagctaaatgcggaagagataagtgctgaaagcatctaagcacgaaacttgccaagagatgagtcatccctgactttaagtcagtaagggttgttgtagactacgacgtagataggttgggtgtgtaagtgatgtgagtcattgagctaaccaatactaattgcccgagaggc

SEQ ID NO: 63 - DNA, 2865 bp, Bordetella bronchiseptica
  modified_base (622): N = A, C, G or T/U
gatcaagcgactaagtgcatatggtggatgccttggcgatcacaggcggatgaaggacgtagtagcctgcgaaaagctgcggggagctggcaaacaagcattgatccgcagatatccgaatggggaaacccacggcaagcggtatccctggctgaatacataggccagtggaggcgaaccgggtgaactgaaacatctcagtagctcgaggaaaagaaatcaaccgagattccgaaagtagtggcgagcgaaatcggaagagcctttacgatttagcattttgcatagtcgaacggaatggaaagtccggccgtagcaggtgatagccctgtagacgaatgcagagtgtggaactaggcgtaagagaagtagggcgggacacgtgaaatcctgtctgaagatggggggaccatcctccaaggctaaatactcgtgatcgaccgatagtgaaccagtaccgtgaggaaaggcgaaaagaaccccggaaggagtgaaatagatcctgaaaccgtatgcatacaacagtcggagcctctttatggggtgacggcgtaccttttgtataatgggtcagcgacttacattcagtggcagcttaaccgaatagggaaggcgtcagaanagcagtccgaatagggcgttccagtcgctgggtgtagacccgaaaccagatgatctacccatggccaggttgaaggcacggtaacacgtgctggaggaccgaacccactagtgttgaaaaactaggggatgagctgtggataggggtgaaaggctaaacaaatctggaaatagctggttctctccgaaaactatttaggtagtgcctcaagtattactgcagggggtagagcactgttatggctagggggtcatggcgacttaccaaaccatggcaaactccgaatacctgcaagtacagcttgggagacagacgaccgggtgctaacgtccggactcaagagggaaacaacccagaccgccagctaaggtcccgaattatcgctaagtgggaaacgaagtgggaaggcatagacagtcaggaggttggcttagaagcagccaccctttaaagaaagcgtaatagctcactgatcgagtcgtcctgcgcggaagatgtaacggctaagcgataaaccgaagctgcgggtgtgcacttttagtgcagcggtaggagagcgttctgtaagcctgcgaaggtggcttgtaaaggctgctggaggtatcagaagtgcgaatgctgacatgagtagccataaagggggtgaaaagccccctcgccgtaagtccaaggtttcctgcgcaacgttcatcggcgcagggtgagtcggcccctaaggcgaggcagagatgcgtagctgatgggaagctggttaatattccagcaccgtcgtacagtgcgatggggggacggatcgcggaaggtcatcagggtgttggacgtccctgttgctgcattgaagatggcgcttaggcaaatccgggcgcgagaatcaagggtgtggcacgagcgagcaagtctcgcgaagtgattggaagtggttccaagaaaagcctctaagcttcagctgtacgagaccgtaccgcaaaccgacacaggtgggacgggatgaatattccaaggcgcttgagagaactcaggagaaggaactcggcaaattgataccgtaacttcgggagaaggtataccctggtagtgtgaagcctgcgcgctgagcatgaaggggtcgcagagaatcggtggctgcgactgtttattaaaaacacagcactctgcaaagacgaaagtcgacgtatagggtgtgacgcctgcccggtgccggaaggttaagtgatggggtgcaagctcttgatcgaagccccggtaaacggcggccgtaactataacggtcctaaggtagcgaaattccttgtcgggtaagttccgacctgcacgaatggcgtaacgatggccacactgtctcctcctgagactcagcgaagttgaagtgtttgtgatgatgcaatctacccgcggctagacggaaagaccccatgaacctttactgtagctttgcattggactgtgaaccggcctgtgtaggataggtgggaggcgcagaactcgagtcgccagattcgagggagccatccttgaaataccaccctggtttgtttgcggttctaaccttggtccgttatccggatcggggacagtgcatggtaggcagtttgactggggcggtctcctcccaaagcgtaacggaggagttcgaaggtacgctaggtacggtcggaaatcgtgctgatagtgcaatggcataagcgtgcttgactgtgagactgacagtgaacaggtgcgaacgggacatagtgatccggtggttctgatggaagggccatcgctcaacggataaaggtactctgggataacaggctgataccgcccaagagttcatatcgacggcggtgtttggcacctcgatgtcggctcatctcatcctggggctgtagccggtccaagggtatgctgttcgccatttaaagaggtacgtgagctgggtttagaaacgtcgtgagacagtttggtccctatctgccgtgggcgttggatacttgaacaggagcctgctcctagtacgagaggaccggagtggacgtacctctggtgtaccggttgtcatgccaatggcattgccgggtagctaagtacggaagagataaccgctgaaggcatctaagcgggaaactcgtctgaagattaggtatcccggggactagatccccctgaagggtcgttcgagaccaggacgttgataggtcgggtgtggaagcgcagtaatgcgttaagctaaccgatactaattgcccgtgaggcttaatcct

SEQ ID NO: 64 - DNA, 2865 bp, Bordetella parapertussis
  modified_base (624): N = A, C, G or T/U
gatcaagcgactaagtgcatatggtggatgccttggcgatcacaggcgatgaaggacgtagtagcctgcgaaaagctgcggggagctggcaaacaagcattgatccgcagatatccgaatggggaaacccacggcaagcggtatccctggctgaatacataggccagtggaggcgaaccgggtgaactgaaacatctcagtagctcgaggaaaagaaatcaaccgagattccgaaagtagtggcgagcgaaatcggaagagcctttacgatttagcattttgcatagtcgaacggaatggaaagtccggccgtagcaggtgatagccctgtagacgaaatgcagagtgtggaactaggcgtaagagaagtagggcgggacacgtgaaatcctgtctgaagatggggggaccatcctccaaggctaaatactcgtgatcgaccgatagtgaaccagtaccgtgaggaaaggcgaaaagaaccccggaaggagtgaaatagatcctgaaaccgtatgcatacaaacagtcggagcctctttatggggtgacggcgtaccttttgtataatgggtcagcgacttacattcagtggcgagcttaaccgaatagggaaggcgtcagaanagcagtccgaatagggcgtccagtcgctgggtgtagacccgaaaccagatgatctacccatggccaggttgaaggcacggtaacacgtcgtggaggaccgaacccactagtgttgaaaaactaggggatgagctgtggataggggtgaaaggctaaacaaatctggaaatagctggttctctccgaaaactatttaggtagtgcctcaagtattactgcagggggtagagcactgttatggctagggggtcatggcgacttaccaaaccatggcaaactccgaatacctgcaagtacagcttgggagacagacgaccgggtgctaacgtccggactcaagagggaaacaacccagaccgccagctaaggtcccgaattatcgctaagtgggaaacgaagtgggaaggcatagacagtcaggaggttggcttagaagcagccaccctttaaagaaagcgtaatagctcactgatcgagtcgtcctgcgcggaagatgtaacggctaagcgataaaccgaagctgcgggtgtgcacttttagtgcagcggtaggagagcgttctgtaagcctgcgaaggtggcttgtaaaggctgctggaggtatcagaagtgcgaatgctgacatgagtagcgataaagggggtgaaaagccccctcgccgtaagtccaaggtttcctgcgcaacgttcatcggcgcagggtgagtcggcccctaaggcgaggcagagatgcgtagctgatgggaagctggttaatattccagcaccgtcgtacagtgcgatggggggacggatcgcggaaggtcatcagggtgttggacgtccctgttgctgcattgaagatggcgcttaggcaaatccgggcgcgagaatcaagggtgtggcacgagcgagcaagtctcgcgaagtgattggaagtggttccaagaaaagcctctaagcttcagctgtacgagaccgtaccgcaaaccgacacaggtgggacgggatgaatattccaaggcgcttgagagaactcaggagaaggaactcggcaaattgataccgtaacttcgggagaaggtataccctggtagtgtgaagcctgcgcgctgagcatgaaggggtcgcagagaatcggtggctgcgactgtttattaaaaacacagcactctgcaaagacgaaagtcgacgtatagggtgtgacgcctgcccggtgccggaaggttaagtgatggggtgcaagctcttgatcgaagccccggtaaacggcggccgtaactataacggtcctaaggtagcgaaattccttgtcgggtaagttccgacctgcacgaatggcgtaacgatggccacactgtctcctcctgagactcagcgaagttgaagtgtttgtgatgatgcaatctacccgcggctagacggaaagaccccatgaacctttactgtagctttgcattggactgtgaaccggcctgtgtaggataggtgggaggcgcagaactcgagtcgccagattcgagggagccatccttgaaataccaccctggtttgtttgcggttctaaccttggtccgttatccggatcggggacagtgcatggtaggcagtttgactggggcggtctcctcccaaagcgtaacggaggagttcgaaggtacgctaggtacggtcggaaatcgtgctgatagtgcaatggcataagcgtgcttgactgtgagactgacagtcgaacaggtgcgaacgggacatagtgatccggtggttctgatggaagggccatcgctcaacggataaaggtactctgggataacaggctgataccgcccaagagttcatatcgacggcggtgtttggcacctcgatgtcggctcatctcatcctggggctgtagccggtccaagggtatgctgttcgccatttaaagaggtacgtgagctgggtttagaaacgtcgtgagacagtttggtccctatctgccgtgggcgttggatacttgaacaggagcctgctcctagtacgagaggaccggagtggacgtacctctggtgtaccggttgtcatgccaatggcattgccgggtagctaagtacggaagagataaccgctgaaggcatctaagcggaaactcgtctgaagattaggtatcccgggactagatccccctgaagggtcgttcgagaccaggacgttgataggtcgggtgtggaagcgcagtaatgcgttaagctaaccgatactaattgcccgtgaggcttgatcct

SEQ ID NO: 65 - DNA, 2864 bp, Bordetella pertussis
  modified_base (624): N = A, C, G or T/U
gatcaagcgactaagtgcatatggtggatgccttggcgatcacaggcgatgaaggacgtagtagcctgcgaaaagctgcggggagctggcaaacaagcattgatccgcagatatccgaatggggaaacccacggcaagcggtatccctggctgaatacataggccagtggaggcgaaccgggtgaactgaaacatctcagtagctcgaggaaaagaaatcaaccgagattccgaaagtagtggcgagcgaaatcggaagagcctttacgatttagcattttgcatagtcgaacggaatggaaagtccggccgtagcaggtgatagccctgtagacgaaatgcagagtgtggaactaggcgtaagagaagtagggcgggacacgtgaaatcctgtctgaagatggggggaccatcctccaaggctaaatactcgtgatcgaccgatagtgaaccagtaccgtgaggaaaggcgaaaagaaccccggaaggagtgaaatagatcctgaaaccgtatgcatacaaacagtcggagcctctttatggggtgacggcgtaccttttgtataatgggtcagcgacttacattcagtggcgagcttaaccgaatagggaaggcgtcagaanagcagtccgaatagggcgtccagtcgctgggtgtagacccgaaaccagatgatctacccatggccaggttgaaggcacggtaacacgtcgtggaggaccgaacccactagtgttgaaaaactaggggatgagctgtggataggggtgaaaggctaaacaaatctggaaatagctggttctctccgaaaactatttaggtagtgcctcaagtattactgcagggggtagagcactgttatggctagggggtcatggcgacttaccaaaccatggcaaactccgaatacctgcaagtacagcttgggagacagacgaccgggtgctaacgtccggactcaagagggaaacaacccagaccgccagctaaggtcccgaattatcgctaagtgggaaacgaagtgggaaggcatagacagtcaggaggttggcttagaagcagccaccctttaaagaaagcgtaatagctcactgatcgagtcgtcctgcgcggaagatgtaacggctaagcgataaaccgaagctgcgggtgtgcacttttagtgcagcggtaggagagcgttctgtaagcctgcgaaggtggcttgtaaaggctgctggaggtatcagaagtgcgaatgctgacatgagtagcgataaagggggtgaaaagccccctcgccgtaagtccaaggtttcctgcgcaacgttcatcggcgcagggtgagtcggcccctaaggcgaggcagagatgcgtagctgatgggaagctggttaatattccagcaccgtcgtacagtgcgatggggggacggatcgcggaaggtcatcagggtgttggacgtccctgttgctgcattgaagatggcgcttaggcaaatccgggcgcgagaatcaagggtgtggcacgagcgagcaagtctcgcgaagtgattggaagtggttccaagaaaagcctctaagcttcagctgtacgagaccgtaccgcaaaccgacacaggtgggacgggatgaatattccaaggcgcttgagagaactcaggagaaggaactcggcaaattgataccgtaacttcgggagaaggtataccctggtagtgtgaagcctgcgcgctgagcatgaaggggtcgcagagaatcggtggctgcgactgtttattaaaaacacagcactctgcaaagacgaaagtcgacgtatagggtgtgacgcctgcccggtgccggaaggttaagtgatggggtgcaagctcttgatcgaagccccggtaaacggcggccgtaactataacggtcctaaggtagcgaaattccttgtcgggtaagttccgacctgcacgaatggcgtaacgatggccacactgtctcctcctgagactcagcgaagttgaagtgtttgtgatgatgcaatctacccgcggctagacggaaagaccccatgaacctttactgtagctttgcattggactgtgaaccggcctgtgtaggataggtgggaggcgcagaactcgagtcgccagattcgagggagccatccttgaaataccaccctggtttgtttgcggttctaaccttggtccgttatccggatcggggacagtgcatggtaggcagtttgactggggcggtctcctcccaaagcgtaacggaggagttcgaaggtacgctaggtacggtcggaaatcgtgctgatagtgcaatggcataagcgtgcttgactgtgagactgacagtcgaacaggtgcgaacgggacatagtgatccggtggttctgatggaagggccatcgctcaacggataaaggtactctgggataacaggctgataccgcccaagagttcatatcgacggcggtgtttggcacctcgatgtcggctcatctcatcctggggctgtagccggtccaagggtatgctgttcgccatttaaagaggtacgtgagctgggtttaaaacgtcgtgagacagtttggtccctatctgccgtgggcgttggatacttgaacaggagcctgctcctagtacgagaggaccggagtggacgtacctctggtgtaccggttgtcatgccaatggcattgccgggtagctaagtacggaagagataaccgctgaaggcatctaagcggaaactcgtctgaagattaggtatcccgggactagatccccctgaagggtcgttcgagaccaggacgttgataggtcgggtgtggaagcgcagtaatgcgttaagctaaccgatactaattgcccgtgaggcttgatcct

SEQ ID NO: 66 - DNA, 2878 bp, Burkholderia cepacia
ggtcaagcgaacaagtgcatgtggtggatgccttggcgatcacaggcgatgaaggacgcggtagcctgcgaaaagctacggggagctggcaaacaagctttgatccgtagatgtccgaatggggaaacccactccttttggagtatccatggctgaatacataggccatgcgaaggaacgcggtgaactgaaacatctaagtaaccgcaggaaaagaaatcaaccgagattcccaaagtagtggcgagcgaaatgggatgagccttgcactctttatttgtattgttagccgaacgctctggaaagtgcggccatagcaggtgatagccctgtaggcgaaaacagtatgaaagaactaggtgtgcgacaagtagggcgggacacgtgaaatcctgtctgaagatggggggaccatcctccaaggctaaatactcgtgatcgaccgatagtgaaccagtaccgtgagggaaaggcgaaaagaaccccgggaggggagtgaaatagatcctgaaaccgcatgcatacaaacagtcggagcctcgtaaggggtgacggcgtaccttttgtataatgggtcagcgacttacgttcagtagcaagcttaaccgtatagggcaggcgtaggaaaggagtccgaatagggcgttcagttgctgggcgtagacccgaaaccaggtgatctatccatggccaggatgaaggtgcggtaacacgtactggaggtccgaacccactaacgttgaaaagttaggggatgagctgtggataggggtgaaaggctaaacaaacctggaaatagctggttctctccgaaaactatttaggtagtgcctcgtgtctcaccttcgggggtagagcactgtcatggttggggggtctattgcagattaccccgccatagcaaactccgaataccgaagagtgcaatcacgggagacagacatcgggtgctaacgtccggtgtcaagagggaaacaacccagaccgccagctaaggtccccaaatatagctaagtgggaaacgaagtgggaaggctaaaacagtcaggaggttggcttagaagcagccaccctttaaagaaagcgtaatagctcactgatcgagtcgtcctgcgcggaagatgtaacggggctaagctatataccgaagctgcggatgcgtgctttgcacgatggtaggagagcgttccgtaagcctgcgaaggtgccttgtaaagggtgctggaggtatcggaagtgcgaatgctgacatgagtagcgataaagggggtgaaaggccccctcgccgtaagcccaaggtttcctacgcaacgttcatcggcgtagggtgagtcggcccctaaggcgaggcagaaatgcgtagctgatgggaagcaggtcaatattcctgcaccattgttagatgcgatggggggacggatcgcggaaggttgtccgggtgttggaagtcccggtcgctgcattggagaaggcgcttaggcaaatccgggcgcagaattcaagggtgtggcgcgagctccttcgggagcgaagcaattggaagtggttccaagaaaagcctctaagcttcagtctaacgatgaccgtaccgcaaaccgacacaggtgggcgagatgagtattctaaggcgcttgagagaactcgggagaaggaactcggcaaattggtaccgtaacttcgggataaggtacgcccttgtagcttgactggcctgcgccaggagggtgaaggggttgcaataaactggtggctgcgactgtttaataaaaacacagcactctgcaaacacgaaagtggacgtatagggtgtgacgcctgcccggtgccggaagattaaatgatggggtgcaagctcttgattgaagtcccggtaaacggcggccgtaactataacggtcctaaggtagcgaaattccttgtcgggtaagttccgacctgcacgaatggcgtaacgatggccacactgtctcctcccgagactcagcgaagttgaagtgtttgtgatgatgcaatctacccgcggctagacggaaagaccccatgaacctttactgtagctttgcattggactttgaaccgatctgtgtaggataggtgggaggctatgaaaccggaacgctagtttcggtggagccgtccttgaaataccaccctggtttgtttgaggttctaaccttggcccgtgatccgggtcggggacagtgcatggtaggcagtttgactggggcggtctcctcccaaagcgtaacggaggagtacgaaggtacgctaggtacggtcggaaatcgtgctgatagtgcaatggcataagcgtgcttaactgcgagaccgacaagtcgagcaggtgcgaaagcaggtcatagtgatccggtggttctgtatggaagggccatcgctcaacggataaaaggtactctggggataacaggctgataccgcccaagagttcatatcgacggcggtgtttggcacctcgatgtcggctcatctcatcctggggctgtagccggtcccaagggtatggctgttcgccatttaaagaggtacgtgagctgggtttaaaacgtcgtgagacagtttggtccctatctgccgtgggcgttggatatttgaagggggctgctcctagtacgagaggaccggagtggacgaacctctggtgtaccggttgtcacgccagtggcatcgccgggtagctatgttcggaagagataaccgctgaaagcatctaagcgggaaactcgccttaagatgagatatccctggggactagatccccttgaagggtcgttcgagaccaggacgttgataggtcaggtgtgtaagcgcagtaatgcgttcagctaactgatactaattgcccgtaaggcttgatcct

SEQ ID NO: 67 - DNA, 2882 bp, Burkholderia mallei
ggtcaagcgaacaagtgcatgtggtggatgccttggcgatcacaggcgatgaaggacgcggtagcctgcgaaaagctacggggagctggcaaacgagctttgatccgtagatgtccgaatggggaaacccggcccttttgggtcatcctagactgaatacataggtctagtgaggcgaacgcggtgaactgaaacatctaagtaaccgcaggaaaagaaatcaaccgagattcccaaagtagtggcgagcgaaatgggaagagcctgtactctttatttgtattgttagccgaacgctctggaaagtgcggccatagcaggtgatagccctgtaggcgaaaacagtatgaaagaactaggtgtacgacaagtagggcgggacacgtgaaatcctgtctgaagatggggggaccatcctccaaggctaaatactcgtgatcgaccgatagtgaaccagtaccgtgagggaaaggcgaaaagaaccccgggaggggagtgaaatagatcctgaaaccgcatgcatacaaacagtcggagcctcttcgggggtgacggcgtaccttttgtataatgggtcagcgacttacgttcagtagcaagcttaaccgaatagggcaggcgtagcgaaagcgagtccgaatagggcgttcagttgctgggcgtagacccgaaaccaggtgatctatccatggccaggatgaaggtgcggtaacacgtactggaggtccgaacccactaacgttgaaaagttaggggatgagctgtggataggggtgaaaggctaaacaaacctggaaatagctggttctctccgaaaactatttaggtagtgcctcgtgtctcaccttcgggggtagagcactgtcatggttggggggtctattgcagattaccccgccatagcaaactccgaataccgaagagtgcaatcacgggagacagacatcgggtgctaacgtccggtgtcaagagggaaacaacccagaccgccagctaaggtccccaaatatggctaagtgggaaacgaagtgggaaggctaaaacagtcaggaggttggcttagaagcagccaccctttaaagaaagcgtaatagctcactgatcgagtcgtcctgcgcggaagatgtaacggggctaagccatataccgaagctgcggatgcgagctagtctcgcatggtaggagagcgttccgtaagcctgcgaaggtgcgttgaaaagcgtgctggaggtatcggaagtgcgaatgctgacatgagtagcgataaagggggtgaaaggccccctcgccgtaagcccaaggtttcctacgcaacgttcatcggcgtagggtgagtcggcccctaaggcgaggcagaaatgcgtagctgatgggaagcaggtcaatattcctgcaccgtcgttagatgcgatggggggacggatcgcggaaggttgtccgggtgttggaagtcccggtcgctgcattggagaaggcgcttaggcaaatccgggcgcaggattcaagggtgtggcgcgagctccttcgggagcgaagcaattggaagtggttccaagaaaagcctctaagcttcagtctaacgatgaccgtaccgcaaaccgacacaggtgggcgagatgagtattctaaggcgcttgagagaactcgggagaaggaactcggcaaattggtaccgtaacttcgggataaggtacgccctggtagcttgactggcctgcgccagaagggtgaaggggttgcaataaactggtggctgcgactgtttaataaaaacacagcactctgcaaacacgaaagtggacgtatagggtgtgacgcctgcccggtgccggaagattaaatgatggggtgcaagctcttgattgaagtcccggtaaacggcggccgtaactataacggtcctaaggtagcgaaattccttgtcgggtaagttccgacctgcacgaatggcgtaacgatggccacactgtctcctcccgagactcagcgaagttgaagtgtttgtgatgatgcaatctacccgcggctagacggaaagaccccatgaacctttactgtagctttgcattggactttgaaccgatctgtgtaggataggtgggaggctatgaaaccggaatgctagtttcggtggagccgtccttgaaataccaccctggtttgtttgaggttctaaccttggcccgtgatccgggtcggggacagtgcatggtaggcagtttgactggggcggtctcctcccaaagcgtaacggaggagtacgaaggtacgctaggtacggtcggaaatcgtgctgatagtgcaatggcataagcgtgcttaactgcgagaccgacaagtcgagcaggtgcgaaagcaggtcatagtgatccggtggttctgtatggaagggccatcgctcaacggataaaaggtactctggggataacaggctgataccgcccaagagttcatatcgacggcggtgtttggcacctcgatgtcggctcatctcatcctggggctgtagccggtcccaagggtatggctgttcgccatttaaagaggtacgtgagctgggtttaaaacgtcgtgagacagtttggtccctatctgccgtgggcgttggaagtttgaagggggctgctcctagtacgagaggaccggagtggacgaacctctggtgtaccggttgtgacgccagtcgcatcgccgggtagctatgttcggaagagataaccgctgaaagcatctaagcgggaaactcgccttaagatgagacttccccggggacttgatccccttgaagggtcgttcgagaccaggacgttgataggtcgggtgtgtaagcgcagtaatgcgttcagctaaccgatactaattgcccgtacggcttgatccta

SEQ ID NO: 68 - DNA, 2882 bp, Burkholderia pseudomallei
ggtcaagcgaacaagtgcatgtggtggatgccttggcgatcacaggcgatgaaggacgcggtagcctgcgaaaagctacggggagctggcaaacgagctttgatccgtagatgtccgaatggggaaacccggcccttttgggtcatcctagactgaatacataggtctagtgaggcgaacgcggtgaactgaaacatctaagtaaccgcaggaaaagaaatcaaccgagattcccaaagtagtggcgagcgaaatgggaagagcctgtactctttatttgtattgttagccgaacgctctggaaagtgcggccatagcaggtgatagccctgtaggcgaaaacagtatgaaagaactaggtgtacgacaagtagggcgggacacgtgaaatcctgtctgaagatggggggaccatcctccaaggctaaatactcgtgatcgaccgatagtgaaccagtaccgtgagggaaaggcgaaaagaaccccgggaggggagtgaaatagatcctgaaaccgcatgcatacaaacagtcggagcctcttcgggggtgacggcgtaccttttgtataatgggtcagcgacttacgttcagtagcaagcttaaccgaatagggcaggcgtagcgaaagcgagtccgaatagggcgttcagttgctgggcgtagacccgaaaccaggtgatctatccatggccaggatgaaggtgcggtaacacgtactggaggtccgaacccactaacgttgaaaagttaggggatgagctgtggataggggtgaaaggctaaacaaacctggaaatagctggttctctccgaaaactatttaggtagtgcctcgtgtctcaccttcgggggtagagcactgtcatggttggggggtctattgcagattaccccgccatagcaaactccgaataccgaagagtgcaatcacgggagacagacatcgggtgctaacgtccggtgtcaagagggaaacaacccagaccgccagctaaggtccccaaatatggctaagtgggaaacgaagtgggaaggctaaaacagtcaggaggttggcttagaagcagccaccctttaaagaaagcgtaatagctcactgatcgagtcgtcctgcgcggaagatgtaacggggctaagccatataccgaagctgcggatgcgagctagtctcgcatggtaggagagcgttccgtaagcctgcgaaggtgcgttgaaaagcgtgctggaggtatcggaagtgcgaatgctgacatgagtagcgataaagggggtgaaaggccccctcgccgtaagcccaaggtttcctacgcaacgttcatcggcgtagggtgagtcggcccctaaggcgaggcagaaatgcgtagctgatgggaagcaggtcaatattcctgcaccgtcgttagatgcgatggggggacggatcgcggaaggttgtccgggtgttggaagtcccggtcgctgcattggagaaggcgcttaggcaaatccgggcgcaggattcaagggtgtggcgcgagcgctctagggcgcgaagcaattggaagtggttccaagaaaagcctctaagcttcagtctaacgatgaccgtaccgcaaaccgacacaggtgggcgagatgagtattctaaggcgcttgagagaactcgggagaaggaactcggcaaattggtaccgtaacttcgggataaggtacgccctggtagcttgactggcctgcgccagaagggtgaaggggttgcaataaactggtggctgcgactgtttaataaaaacacagcactctgcaaacacgaaagtggacgtatagggtgtgacgcctgcccggtgccggaagattaaatgatggggtgcaagctcttgattgaagtcccggtaaacggcggccgtaactataacggtcctaaggtagcgaaattccttgtcgggtaagttccgacctgcacgaatggcgtaacgatggccacactgtctcctcccgagactcagcgaagttgaagtgtttgtgatgatgcaatctacccgcggctagacggaaagaccccatgaacctttactgtagctttgcattggactttgaaccgatctgtgtaggataggtgggaggctatgaaaccggaacgctagtttcggtggagccgtccttgaaataccaccctggtttgtttgaggttctaaccttggcccgtgatccgggtcggggacagtgcatggtaggcagtttgactggggcggtctcctcccaaagcgtaacggaggagtacgaaggtacgctaggtacggtcggaaatcgtgctgatagtgcaatggcataagcgtgcttaactgcgagaccgacaagtcgagcaggtgcgaaagcaggtcatagtgatccggtggttctgtatggaagggccatcgctcaacggataaaaggtactctggggataacaggctgataccgcccaagagttcatatcgacggcggtgtttggcacctcgatgtcggctcatctcatcctggggctgtagccggtcccaagggtatggctgttcgccatttaaagaggtacgtgagctgggtttaaaacgtcgtgagacagtttggtccctatctgccgtgggcgttggaagtttgaagggggctgctcctagtacgagaggaccggagtggacgaacctctggtgtaccggttgtgacgccagtcgcatcgccgggtagctatgttcggaagagataaccgctgaaagcatctaagcgggaaactcgccttaagatgagacttccccggggacttgatccccttgaagggtcgttcgagaccaggacgttgataggtcgggtgtgtaagcgcagtaatgcgttcagctaaccgatactaattgcccgtacggcttgatccta

SEQ ID NO: 69 - DNA, 2890 bp, Neisseria gonorrhoeae
ggtcaagtgaataagtgcatcaggcggatgccttggcgatgataggcgacgaaggacgtgtaagcctgcgaaaagcgcgggggagctggcaataaagctatgattccgcgatgtccgaatggggaaacccactgcattctgtgcagtatcctaagttgaatacataggcttagagaagcgaacccggagaactgaaccatctaagtacccggaggaaaagaaatcaaccgagattccgcaagtagtggcgagcgaacgcggaggagcctgtacgtaataactgtcgagatagaagaacaagctgggaagcttgaccatagcgggtgacagtcccgtattcgaaatctcaacagcggtactaagcgtacgaaaagtagggcgggacacgtgaaatcctgtctgaatatggggggaccatcctccaaggctaaatactcatcatcgaccgatagtgaaccagtaccgtgagggaaaggcgaaaagaaccccgggagggaagtgaaacagaacctgaaacctgatgcatacaaacagtgggagcgccctagtggtgtgactgcgtaccttttgtataatgggtcaacgacttacattcagtagcgagcttaaccggataggggaggcgtagggaaaccgagtcttaatagggcgatgagttgctgggtgtagacccgaaaccgagtgatctatccatggtcaggttgaaggtgccgtaacaggtactggaggaccgaacccacgcatgttgcaaaatgcggggatgagctgtgggtaggggtgaaaggctaaacaaactcggagatagctggttctccccgaaaactatttaggtagtgcctcgagcaagacactgatgggggtaaagcactgttatggctagggggttattgcaacttaccaacccatggcaaactcagaataccatcaagtggttcctcgggagacagacagcgggtgctaacgtccgttgtcaagagggaaacaacccagaccgccggctaaggtcccaaatgatagattaagtggtaaacgaagtgggaaggcacagacagccaggatgttggcttagaagcagccatcatttaaagaaagcgtaatagctcactggtcgagtcgtcctgcgcggaagatgtaacggggctcaaatctataaccgaagctgcggatgccggtttaccggcatggtaggggagcgttctgtaggctgatgaaggtgcattgtaaagtgtgctggaggtatcagaagtgcgaatgttgacatgagtagcgataaagcgggtgaaaagcccgctcgccgaaagcccaaggtttcctacgcaacgttcatcggcgtagggtaagtcggcccctaaggcgaggcagaaatgcgtagtcgatgggaaacaggttaatattcctgtacttgattcaaatgcgatgtggggacggagaaggttaggttggcaagctgttggaatagcttgtttaagccggtaggtggaagacttaggcaaatccgggttttcttaacaccgagaagtgatgacgagtgtctacggacacgaagcaaccgataccacgcttccaggaaaagccactaagcttcagtttgaatcgaaccgtaccccaaaccgacacaggtgggtaggatgagaattctaaggcgcttgagagaactcgggagaaggaactcggcaaattgataccgtaacttcgggagaaggtatgccctctaaggttaaggacttgctccgtaagccccggagggtcgcagagaataggtggctgcgactgtttattaaaaacacagcactctgccaacacgaaagtggacgtatagggtgtgacgcctgcccggtgccggaaggttaattgaagatgtgcaagcatcggatcgaagccccggtaaacggcggccgtaactataacggtcctaaggtagcgaaattccttgtcgggtaagttccgacccgcacgaatggcgtaacgatggccacactgtctcctcccgagactcagcgaagttgaagtggttgtgaagatgcaatctacccgctgctagacggaaagaccccgtgaacctttactgtagctttgcattggactttgaagtcacttgtgtaggataggtgggaggcttggaagcagagacgccagtctctgtggagtcgtccttgaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgggcaggtgcgaaagcaggacatagtgatccggtggttctgtatggaagggccatcgctcaacggataaaaggtactccggggataacaggctgattccgcccaagagttcatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtggcgttggaagtttgacggggctgctcctagtacgagaggaccggagtggacgaacctctggtgtaccggttgtaacgccagttgcatagccgggtagctaagttcggaagagataagcgctgaaagcatctaagcgcgaaactcgcctgaagatgagacttcccttgcggtttaaccgcactaaagggtcgttcgagaccaggacgttgataggtggggtgtggaagcgcggtaacgcgtgaagctaacccatactaattgcccgtgaggcttgactct

SEQ ID NO: 70 - DNA, 2891 bp, Neisseria meningitidis
gtcaagtgaataagtgcatcaggtggatgccttggcgatgataggcgacgaaggacgtgtaagcctgcgaaaagcgcgggggagctggcaataaagcaatgatcccgcgatgtccgaatggggaaacccactgcattctgtgcagtatcctaagttgaatacatagacttagagaagcgaacccggagaactgaaccatctaagtacccggaggaaaagaaatcaaccgagattccgcaagtagtggcgagcgaacgcggaggagcctgtacgtaataactgtcgagatagaagaacaagctgggaagcttgaccatagtgggtgacagtcccgtattcgaaatctcaacagcggtactaagcgtacgaaaagtagggcggggcacgtgaaatcctgtctgaatatggggggaccatcctccaaggctaaatactcatcatcgaccgatagtgaaccagtaccgtgagggaaaggcgaaaagaaccccgggaggggagtgaaacagaacctgaaacctgatgcatacaaacagtgggagcgccctagtggtgtgactgcgtaccttttgtataatgggtcaacgacttacattcagtagcgagcttaaccgaataggggaggcgtagggaaaccgagtcttaatagggcgatgagttgctgggtgtagacccgaaaccgagtgatctatccatggccaggttgaaggtgccgtaacaggtactggaggaccgaacccacgcatgttgcaaaatgcggggatgagctgtggataggggtgaaaggctaaacaaactcggagatagctggttctccccgaaaactatttaggtagtgcctcgagcaagacactgatgggggtaaagcactgttatggctagggggttattgcaacttaccaacccatggcaaactaagaataccatcaagtggttcctcgggagacagacagcgggtgctaacgtccgttgtcaagagggaaacaacccagaccgccagctaaggtcccaaatgatagattaagtggtaaacgaagtgggaaggcccagacagccaggatgttggcttagaagcagccatcatttaaagaaagcgtaatagctcactggtcgagtcgtcctgcgcggaagatgtaacggggctcaaatctataaccgaagctgcggatgccggtttaccggcatggtaggggagcgttctgtaggctgatgaaggtgcattgtaaagtgtgctggaggtatcagaagtgcgaatgttgacatgagtagcgataaagcgggtgaaaagcccgctcgccgaaagcccaaggtttcctgcgcaacgttcatcggcgtagggtgagtcggcccctaaggcgaggcagaaatgcgtagtcgatgggaaacaggttaatattcctgtacttgattcaaatgcgatgtggggacggagaaggttaggttggcaagctgttggaatagcttgtttaagccggtaggtggaagacttaggcaaatccgggtcttcttaacaccgagaagtgacgacgagtgtctacggacacgaagcaaccgataccacgcttccaggaaaagccactaagcttcagtttgaatcgaaccgtaccgcaaaccgacacaggtgggcaggatgagaattctaaggcgcttgagagaactcaggagaaggaactcggcaaattgataccgtaacttcgggagaaggtatgccctctaaggttaaggacttgctccgtaagccccggagggtcgcagagaataggtggctgcgactgtttattaaaaacacagcactctgctaacacgaaagtggacgtatagggtgtgacgcctgcccggtgctggaaggttaattgaagatgtgagagcatcggatcgaagccccagtaaacggcggccgtaactataacggtcctaaggtagcgaaattccttgtcgggtaagttccgacccgcacgaatggcgtaacgatggccacactgtctcctcctgagactcagcgaagttgaagtggttgtgaagatgcaatctacccgctgctagacggaaagaccccgtgaacctttactgtagctttgcattggactttgaagtcacttgtgtaggataggtgggaggcttagaagcagagacgccagtctctgtggagccgtccttgaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgagcaggtgcgaaagcaggacatagtgatccggtggttctgtatggaagggccatcgctcaacggataaaaggtactccggggataacaggctgattccgcccaagagttcatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtgggcgttggaagtttgacgggggctgctcctagtacgagaggaccggagtggacgaacctctggtgtaccggttgtaacgccagttgcatagccgggtagctaagttcggaagagataagcgctgaaagcatctaagcgcgaaactcgcctgaagatgagacttcccttgcggtttaaccgcactaaagagtcgttcgagaccaggacgttgataggtggggtgtggaagcgcggtaacgcgtgaagctaacccatactaattgctcgtgaggcttgactct

SEQ ID NO: 71 - DNA, 2891 bp, Pseudomonas aeruginosa
ggtcaagtgaagaagcgcatacggtggatgccttggcagtcagaggcgatgaaagacgtggtagcctgcgaaaagcttcggggagtcggcaaacagactttgatccggagatctctgaatgggggaacccacctaggataacctaggtatcttgtactgaatccataggtgcaagaggcgaaccaggggaactgaaacatctaagtaccctgaggaaaagaaatcaaccgagattcccttagtagtggcgagcgaacggggattagcccttaagcttcattgattttagcggaacgctctggaaagtgcggccatagtgggtgatagccccgtacgcgaaaggatctttgaagtgaaatcgagtaggacggagcacgagaaactttgtctgaacatggggggaccatcctccaaggctaaatactactgactgaccgatagtgaaccagtaccgtgagggaaaggcgaaaagaaccccggagaggggagtgaaatagaacctgaaaccgtatgcgtacaagcagtgggagcctacttgttaggtgactgcgtaccttttgtataatgggtcagcgacttatattcagtggcaagcttaaccgtatagggtaggcgtagcgaaagcgagtcttaatagggcgtttagtcgctgggtatagacccgaaaccgggcgatctatccatgagcaggttgaaggttaggtaacactgactggaggaccgaacccactcccgttgaaaaggtaggggatgacttgtggatcggagtgaaaggctaatcaagctcggagatagctggttctcctcgaaagctatttaggtagcgcctcatgtatcactctggggggtagagcactgtttcggctagggggtcatcccgacttaccaaaccgatgcaaactccgaatacccagaagtgccgagcatgggagacacacggcgggtgctaacgtccgtcgtgaaaagggaaacaacccagaccgccagctaaggtcccaaagttgtggttaagtggtaaacgatgtgggaaggcttagacagctaggaggttggcttagaagcagccaccctttaaagaaagcgtaatagctcactagtcgagtcggcctgcgcggaagatgtaacggggctcaaaccacacaccgaagctgcgggtgtcacgtaagtgacgcggtagaggagcgttctgtaagcctgtgaaggtgagttgagaagcttgctggaggtatcagaagtgcgaatgctgacatgagtaacgacaatgggtgtgaaaaacacccacgccgaaagaccaagggttcctgcgcaacgttaatcgacgcagggttagtcggttcctaaggcgaggctgaaaagcgtagtcgatgggaaacaggttaatattcctgtacttctggttactgcgatggagggacggagaaggctaggccagcttggcgttggttgtccaagtttaaggtggtaggctgaaatcttaggtaaatccggggtttcaaggccgagagctgatgacgagtcgtcttttagatgacgaagtggttgatgccatgcttccaagaaaagcttctaagcttcaggtaaccaggaaccgtaccccaaaccgacacaggtggtcgggtagagaataccaaggcgcttgagagaactcgggtgaaggaactaggcaaaatggcaccgtaacttcgggagaaggtgcgccggctagggtgaaggatttactccgtaagctctggctggtcgaagataccaggccgctgcgactgtttattaaaaacacagcactctgcaaacacgaaagtggacgtatagggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaacgatggcggcgctgtctccacccgagactcagtgaaattgaaatcgctgtgaagatgcagtgtatccgcggctagacggaaagaccccgtgaacctttactgtagctttgcactggactttgagcctgcttgtgtaggataggtgggaggctttgaagcgtggacgccagttcgcgtggagccatccttgaaataccaccctggcatgcttgaggttctaactctggtccgtaatccggatcgaggacagtgtatggtgggcagtttgactggggcggtctcctcctaaagagtaacggaggagtacgaaggtgcgctcagaccggtcggaaatcggtcgcagagtataaaggcaaaagcgcgcttgactgcgagacagacacgtcgagcaggtacgaaagtaggtcttagtgatccggtggttctgtatggaagggccatcgctcaacggataaaaggtactccggggataacaggctgataccgcccaagagttcatatcgacggcggtgtttggcacctcgatgtcggctcatcacatcctggggctgaagccggtcccaagggtatggctgttcgccatttaaagtggtacgcgagctgggtttagaacgtcgtgagacagttcggtccctatctgccgtggacgtttgagatttgagaggggctgctcctagtacgagaggaccggagtggacgaacctctggtgttccggttgtcacgccagtggcattgccgggtagctatgttcggaaaagataaccgctgaaagcatctaagcgggaaacttgcctcaagatgagatctcactgggaacttgattcccctgaagggccgtcgaagactacgacgttgataggctgggtgtgtaagcgttgtgaggcgttgagctaaccagtactaattgcccgtgaggcttgaccat

SEQ ID NO: 72 - DNA, 2886 bp, Vibrio cholerae
ggttaagtgactaagcgtacacggtggatgcctgggcagtcagaggcgatgaaggacgtactaacttgcgataagcgcagataaggcagtaagagccgtttgagtctgcgatttccgaatggggaaacccaactgcataagcagttactgttaactgaatacataggttaacagagcaaaccgggggaactgaaacatctaagtaccccgaggagaagaaatcaaccgagattccggtagtagcggcgagcgaacctggattagcccttaagcactcggtgaagtaggtgaacaagctggaaagcttggcgatacagggtgatagccccgtaaccgacgcttcatcgagcgtgaaatcgagtagggcgggacacgtgatatcctgtctgaatatggggggaccatcctccaaggctaaatactcctgactgaccgatagtgaaccagtaccgtgaggaaaggcgaaaagaacccctgtgaggggagtgaaatagaacctgaaaccgtgtacgtacaagcagtaggagcaccttcgtggtgtgactgcgtaccttttgtataatgggtcagcgacttatattcagtggcaaggttaaccgtataggggagccgtagcgaaagcgagtcttaactgggcgctcagtctctggatatagacccgaaaccgggtgatctagccatgggcaggttgaaggttgagtaacatcaactggaggaccgaaccgactaatgttgaaaaattagcggatgacttgtggctaggggtgaaaggccaatcaaactcggagatagctggttctccccgaaagctatttaggtagcgcctcggacgaatactactgggggtagagcactgttaaggctagggggtcatcccgacttaccaaccctttgcaaactccgaataccagtaagtactatccgggagacacacggcgggtgctaacgtccgtcgtggagagggaaacaacccagaccgccagctaaggtcccaaagtattgctaagtgggaaacgatgtgggaaggctcagacagctaggatgttggcttagaagcagccatcatttaaagaaagcgtaatagctcactagtcgagtcggcctgcgcggaagatgtaacggggctaagcaatacaccgaagctgcggcaatatcttttagatattgggtaggggagcgttctgtaagccgttgaaggtgaatcgtaaggtttgctggaggtatcagaagtgcgaatgctgacatgagtaacgacaaagggggtgaaaaacctcctcgccggaagaccaagggttcctgtccaacgttaatcggggcagggtgagtcgacccctaaggtgaggccgaaaggcgtaatcgatgggaaacgggttaatattcccgtacttctgactattgcgatggggggacggagaaggctaggtgggccaggcgacggttgtcctggttcaagtgcgtaggcttgagagttaggtaaatccggctctctctaaggctgagacacgacgtcgagctactacggtagtgaagtcattgatgccatgcttccaggaaaagcctctaagcttcagatagtcaggaatcgtaccccaaaccgacacaggtggtcgggtagagaataccaaggcgcttgagagaactcgggtgaaggaactaggcaaaatggtaccgtaacttcgggagaaggtacgctcttgatggtgaagtccctcgcggatggagctgacgagagtcgcagataccaggtggctgcaactgtttattaaaaacacagcactgtgcaaaatcgcaagatgacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccacgctgtctccacccgagactcagtgaaattgaaatcgctgtgaagatgcagtgtacccgcggctagacggaaagaccccgtgaacctttactacagcttggcactgaacattgaacctacatgtgtaggataggtgggaggctatgaagacgtgacgccagttgcgttggagccgtccttgaaataccacccttgtatgtttgatgttctaacttagacccgttatccgggttgaggacagtgcctggtgggtagtttgactggggcggtctcctcccaaagagtaacggaggagcacgaaggtgggctaatcacggttggacatcgtgaggttagtgcaatggcataagcccgcttaactgcgagaatgacggttcgagcaggtgcgaaagcaggtcatagtgatccggtggttctgtatggaagggccatcgctcaacggataaaaggtactccggggataacaggctgataccgcccaagagttcatatcgacggcggtgtttggcacctcgatgtcggctcatcacatcctggggctgaagtcggtcccaagggtatggctgttcgccatttaaagtggtacgcgagctgggtttagaacgtcgtgagacagttcggtccctatctgccgtgggcgttggaagattgaagggggctgctcctagtacgagaggaccggagtggacgaacctctggtgttcgggttgtgtcgccagacgcattgcccggtagctaagttcggaattgataagcgctgaaagcatctaagcgcgaagcgagccctgagatgagtcttccctgacagtttaactgtcctaaagggttgttcgagactagaacgttgataggcagggtgtgtaagcgttgtgaggcgttgagctaacctgtactaattgcccgtgaggcttaaccat

SEQ ID NO: 73 - DNA, 2906 bp, Yersinia enterocolitica
  modified_base (1168)..(1178): N = A, C, G or T/U
ggttaagcgaccaagcgtacacggtggatgcctaggcagtcagaggcgatgaaggacgtgctaatctgcgaaaagcgtcggtaaggtgatatgaaccgttataaccgacgatacccgaatggggaaacccagtgcaattcgttgcactattgcatggtgaatacatagccatgcaaggcgaaccgggggaactgaaacatctaagtaccccgaggaaaagaaatcaaccgagattcccccagtagcggcgagcgaacggggaggagcccagaacctgaatcagcgtatgtgttagtggaagcgtctggaaagtcgcacggtacagggtgatagtcccgtacacaaaaatgcatatgttgtgagttcgatgagtagggcgggacacgtgacatcctgtctgaatatggggggaccatcctccaaggctaaatactcctgactgaccgatagtgaaccagtaccgtgagggaaaggcgaaaagaaccccggcgaggggagtgaaacagaacctgaaaccgtgtacgtacaagcagtgggagcaccttcgtggtgtgactgcgtaccttttgtataatgggtcagcgacttatattttgtagcaaggttaaccgaataggggagccgtagggaaaccgagtcttaactgggcgaatagttgcaaggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaactcatcttcgggggtagagcactgtttcggctagggggtcatcccgacttaccaaaccgatgcaaactccgaataccgaagaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttagaagcagccatcatttaaagaaagcgtaatagctcactggtcgagtcggcctgcgcggaagatgtaacggggctaaaccatgcaccgaagctgcggcagcgnnnnnnnnnnnnnnnnnnnnnggggagcgttctgtaagccgttgaaggtgacctgtgagggttgctggaggtatcagaagtgcgaatgctgacataagtaacgataatgcgggtgaaaaacccgcacgccggaagaccaagggttcctgtccaacgttaatcggggcagggtgagtcgacccctaaggcgaggctgaaaggcgtagtcgatgggaaacaggttaatattcctgtacttggtgttactgcgaaggggggacggagaaggctatgctagccgggcgacggttgtcccggtttaagcatgtaggcggagtgaccaggtaaatccggttgcttatcaacgctgaggtgtgatgacgagtcactacggtgatgaagtagttgatgccatgcttccaggaaaagcctctaagcatcaggtaacatgaaatcgtaccccaaaccgacacaggtggtcaggtagagaatactcaggcgcttgagagaactcgggtgaaggaactaggcaaaatggtgccgtaacttcgggagaaggcacgctgacacgtaggtgaagcggtttacccgtggagctgaagtcagtcgaagataccagctggctgcaactgtttattaaaaacacagcactgtgcaaacacgaaagtggacgtatacggtgtgacgcctgcccggtgctggaaggttaattgatggggtcagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggcatagaagtgtggacgccagtctgcatggagccaaccttgaaataccaccctttaatgtttgatgttctaactcggccccgtaatccggggtgaggacagtgtcaggtgggtagtttgactggggcggtctcctcccaaagagtaacggaggagcacgaaggttagctaatcacggtcggacatcgtgaggttagtgcaaaggcataagctagcttcactgcgagagtgacggctcgagcaggtacgaaagtaggtcttagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgytggarraytgagrggggctgctcctagtacgagaggaccggagtggacgmatcactggtgttcgggttgtcatgccaatggcaytgcccggtagctaaatkcggaagagataasygctgaaagcatctaagcrsgaaacttgccycgagatgagttctccctgagactacaagtctcctgaaggaacgttgaagacgacgacgttgataggcygggtgtgtaagcgcgagttggcgttgagctaaccggtactaatgaaccgtgaggcttaacctt

(19) United States
(12) Patent Application Publication
(10) Pub. No.: US 2003/0175709 A1
(43) Pub. Date: Sep. 18, 2003

(54) Method and system for depleting rRNA populations

(76) Inventors: George L. Murphy, Austin, TX (US); J. Penn Whitley, Austin, TX (US)

Correspondence Address: FULBRIGHT & JAWORSKI L.L.P., A REGISTERED LIMITED LIABILITY PARTNERSHIP, 600 CONGRESS AVENUE, SUITE 2400, AUSTIN, TX, 78701, US

(21) Appl. No.: 10/029,397

(22) Filed: Dec. 20, 2001

(51) Int. Cl. C12Q 1/68; C07H 21/02; C12P 19/34

(52) U.S. Cl. 435/6; 435/91.2; 536/23.1

(57) ABSTRACT

The present invention concerns a system for isolating, depleting, or separating a targeted nucleic acid, such as rRNA, from a sample comprising targeted and nontargeted nucleic acids. It effects a way of enriching for nontargeted nucleic acids, such as mRNAs. The invention further concerns methods of implementing the system and kits for implementing the system, which involves at least one bridging nucleic acid comprising 1) a targeting region complementary to a region on the targeted nucleic acid and 2) a bridging region complementary to the capture region of a capture nucleic acid that comprises a nonreactant structure. The nonreactant structure can be used to isolate the hybridizing molecules after incubation under conditions that allows hybridization.

### BACKGROUND OF THE INVENTION

1. Field of the Invention

The present invention relates generally to the fields of molecular biology and microbial pathogenesis. More particularly, it concerns methods, compositions, and kits for isolating, depleting, separating a targeted nucleic acid population from other nucleic acid populations as a means for enriching those other nucleic acid population(s). More particularly, it concerns methods, compositions, and kits for enriching mRNA populations by depleting eukaryotic and/or prokaryotic rRNA from a sample using engineered bridging and capture nucleic acid molecules.

2. Description of Related Art

The ongoing efforts in microbial genome sequencing will enable unprecedented advances in our understanding of microbes and host-microbe interactions. Dozens of prokaryotic genomes, including those of numerous human pathogens, have been completely sequenced, and many others are in progress. Consequently, a renewal of focus and energy has emerged in the fields of microbial evolution, microbial pathogenesis, and infectious diseases. The potential impact of genomics on these disciplines is the subject of several recent reviews (Cummings et al., 2000; Cornelis et al., 2001; Fox et al., 2001; Current Opinion in Microbiology). For host-microbe interactions, the ability to measure the expression of every single gene in a microorganism will make possible studies of such complex interactions as the global regulation of virulence factors and the mechanisms of response to host cells and their microenvironment. Scientists will also be able to evaluate the complete repertoire of host cell gene expression in response to the pathogen. Undoubtedly, novel interactions and responses between microbes and their hosts will be discovered, leading to a more complete picture of infectious diseases and how to control them.

In the past decade, researchers studying bacteria developed several novel approaches to evaluate global gene transcription in response to environmental stimuli, including host-microbe interactions. Prior to the era of genome sequencing, Chuang et al. (Chuang et a., 1993) used an ordered set of E. coli lambda library clones to evaluate global transcription responses of E. coli. Other groups employed subtractive hybridization and differential screening to evaluate induction of gene expression in Mycobacterium avium after phagocytosis by macrophages (Plum et al., 1994) or in Pyrococcus grown under specific environmental conditions (Robinson et al., 1994). Researchers further developed this approach with an elegant procedure for the selective capture of transcribed sequences (SCOTS) (Graham et al., 1999). At the same time, many scientists bypassed library construction altogether and used using differential display (Liang et al., 1995) to discover genes that are transcribed differently under various growth conditions. Although useful in certain circumstances, differential display is frequently a hit-or-miss prospect and gives no information on global transcription. More recently, serial analysis of gene expression (SAGE) (Velculescu et al., 1995) emerged as a method for analyzing the complete transcriptome of a cell. SAGE, like differential display, can be useful but requires large amounts of nucleic acid sequencing. Not unexpectedly, for organisms whose genomes have been sequenced, array analysis is emerging as the method of choice for global gene expression studies with bacteria. Macroarrays (filter-based arrays) and microarrays (slide-based arrays) of complete genomes have made possible the simultaneous expression analysis of thousands of genes. The advent of microarray technology has already enabled analyses of the host response to interactions with pathogenic organisms (Cummings et al., 2000). Similarly, microarray analysis and other methods have been used to evaluate gene expression in bacteria grown under different environmental conditions in vitro.

The application of array analysis to gene expression profiling in prokaryotes was an immediate outgrowth of similar studies with eukaryotic organisms, occurring only within the past two to three years. Infectious disease researchers have already begun applying microarray analysis to the study of complex host-microbe interactions. To date, such analyses of host-microbe interactions have been limited to the evaluation of host cell responses to bacteria or viruses. Bordetella pertussis, Listeria monocytogenes, Neisseria meningitidis, Pseudomonas aeruginosa, Legionella pneumophila, Salmonella dublin, and Staphylococcus aureus are among the bacterial pathogens whose effects on host cell gene expression have been evaluated with microarrays. Array analyses of eukaryotic host cell transcription are feasible because of the ability to isolate polyadenylated mRNAs from eukaryotic cells and to specifically label mRNAs by oligo dT-primed cDNA synthesis.

Although it has been alluded to in the literature (Cummings et al, 2000; Rappuoli, 2000), complete genome array expression analyses of bacteria in response to interactions with host cells have not been widely published, if at all. Studies that examine the global bacterial gene response in the presence of host cells will require the development of tools to enable the efficient isolation, enrichment, and labeling of bacterial mRNAs (Cummings et al., 2000; Graham et al., 1999; Gingeras et al., 2000; Graham et al., 2001).

However, technical limitations of current methods available for purification and evaluation of bacterial mRNAs preclude these types of whole genome analysis. To realize the full potential of the genomics revolution, methods for purifying mRNAs from total bacterial RNA populations and particularly from mixtures of host cell and bacterial RNA need to be developed.

Isolating sufficient quantities of high quality bacterial mRNA is perhaps the most demanding technical requirement impeding analyses of bacterial gene expression in the presence of host cells. A small percentage of bacterial mRNAs may be A-tailed, but these are targeted for degradation and tend to be unstable. As a result, the commonly used method for mRNA purification with eukaryotic cells, oligo-dT capture, is ineffective.

Only a few studies have described methods for enriching or purifying bacterial mRNAs. Several groups (Plum et al., 1994; Robinson et al., 1994; Su et al., 1998) have used rRNA subtraction to enrich for bacterial mRNAs. These procedures involved hybridization of rRNAs to biotinylated plasmid containing rRNA genes or to biotinylated antisense rRNAs followed by streptavidin capture and removal. This yields some benefits, but it requires fairly large amounts of plasmids or antisense RNA. Biotinylation of large amounts of DNA or RNA is often tricky and can be prohibitively expensive if biotin-modified nucleotides are incorporated during antisense RNA synthesis. In general, these methods have not seen widespread use. As mentioned above, Graham and Clarke-Curtiss (Graham et al., 1999) went further in enriching for mycobacterial mRNAs with SCOTS. The SCOTS procedure is effective for detecting genes specifically expressed in the presence of host cells but is hampered by being a multi-step procedure that requires production of normalized double-stranded cDNA, PCR, differential hybridization, and cDNA capture. In addition to these methods, researchers have developed methods to polyadenylate bacterial mRNAs, thereby allowing for their purification by oligo dT-capture. Amara and Vijaya (Amara et al., 1997) demonstrated that mRNAs in purified polysomes can be specifically polyadenylated and purified by oligo-dT capture. Wendisch et al. (Wendisch et al., 2001) showed that the same process can be carried out with crude cell extracts. Several shortcomings are associated with the polyadenylation approach. Different mRNAs may be polyadenylated to different extents or not at all depending on the structure of their 5′ and 3′ ends (Feng et al., 2000). Polyadenylation in a cell lysate, followed by purification of RNA, will require inactivation of cellular RNAses so that transcripts are not degraded during the polyadenylation reaction. Optimizing the reaction to work reproducibly in many different bacterial cell lysates would likely be very difficult. Despite many worthy attempts, simple and universal procedures for bacterial mRNA enrichment, especially in the presence of host cell RNA, remain elusive. Thus, there remains a continued need for improvements in mRNA enrichment and/or the depletion of other RNA populations.

### SUMMARY OF THE INVENTION

The present invention involves a system that allows for the isolation, separation, and depletion of a population of nucleic acid molecules. The system involves components that may be used to implement methods for isolating, separating, or depleting a targeted nucleic acid. Such components may also be included in kits of the invention.

In embodiments of the invention, a population of nucleic acids may be targeted for isolation, separation, or depletion. Such a nucleic acid is referred to as “targeted nucleic acid” or “targeted nucleic acid molecule.” Alternatively, it may be referred to as a “nucleic acid target.” In particular embodiments of the invention, the targeted nucleic acid is rRNA. In alternative embodiments, the targeted nucleic acid is mRNA, tRNA, or DNA including, cDNA and genomic DNA. The targeted nucleic acid may be in a sample, which is a composition that is suspected of containing the targeted nucleic acid. In some embodiments, the sample is obtained from or includes prokaryotes or eukaryotes or both. The sample may be cells, tissues, organs, and lysates, fractionations, or portions thereof. Furthermore, the targeted nucleic acid is targeted via a “targeting region” in the targeted nucleic acid. A “targeted region” refers to a region of the targeted nucleic acid that is complementary with the targeting region of a bridging nucleic acid and that allows the targeted nucleic acid to be separated from other non-targeted nucleic acid populations.

In embodiments in which the targeted nucleic acid is rRNA, the rRNA may be the SS, 16S, or 23S rRNA from prokaryotes, though it may be any rRNA species from a prokaryotes. It is specifically contemplated that nucleic acids may be targeted in Gram positive bacteria and Gram negative bacteria. In further embodiments, the targeted rRNA is 5.8S, 17S or 18S, or 28S rRNA (referred to as “types of rRNA”) from a eukaryote. It is further contemplated that tRNA may be a targeted nucleic acid population either by itself or in combination with any of the targeted nucleic acids described herein. A non-limiting list of targeted rRNAs from various organisms is provided in a later section and is contemplated to be part of the invention.

In embodiments of the invention, the system involves a bridging nucleic acid, a capture nucleic acid, and a targeted nucleic acid, as shown, for example, in FIG. 1. While in many embodiments of the invention it is contemplated that the bridging nucleic acid and the capture nucleic acid are oligonucleotides, it is specifically contemplated that they may be polynucleotides as well. Thus, any embodiment involving an oligonucleotide may be implemented with a polynucleotide. Bridging nucleic acids, capture nucleic acids, and targeted nucleic acids of the invention may include, be at least or be at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 or more residues in length.

Furthermore, a “bridging nucleic acid” is a nucleic acid molecule that comprises a bridging region and a targeting region, while a “capture nucleic acid” is a nucleic acid molecule that comprises a capture region. It is contemplated that bridging, targeting, and capture regions of the invention may be, be at least or be at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 residues in length.

A “bridging nucleic acid” refers to a molecule that includes nucleic acid residues or analogs and that includes at least one targeting region and at least one bridging region. A “targeting region” refers to a region of the molecule that is involved in targeting a particular nucleic acid or nucleic acid population and is thus complementary to all or part of the sequence of the targeted nucleic acid. It is further contemplated that more than one targeting region may be included in a bridging nucleic acid. The bridging nucleic acid may include or have up to 2, 3, 4, 5, 6, 7, 8, 9, 10, or more targeting regions. When there are multiple targeting regions, it is contemplated that the regions may be complementary to different, nonoverlapping sequences from the same targeted nucleic acid or they may be complementary to similar or overlapping sequences from the same targeted nucleic acid, or they may be complementary to sequences in different targeted nucleic acids. While mRNA may be targeted, it is specifically contemplated that mRNA is not targeted and thus the targeting region does not have a stretch of polypyrimidine residues, such as poly-T or poly-U to hybridize to the poly-A tail of eukaryotic mRNA. Also considered part of the invention is using single or multiple bridging nucleic acids to deplete an rRNA population. In some embodiments, a single bridging nucleic acid may contain one or more targeting regions that are complementary to different types of rRNA (“types” refer to sizes based on intact lengths). Thus, in some embodiments, the largest type of rRNA may be targeted (“largest” refers to longest nucleic acid molecule when intact, even though molecules that are no longer intact may also be targeted if they retain the sequence that is complementary to all or part of a targeting region). In still further embodiments, the second largest rRNA or the first and second largest rRNA types may be targeted by a single bridging nucleic acid with targeting regions to each or to more than one nucleic acid, each with a targeting region to a different type of rRNA. In still further embodiments, a bridging nucleic acid has a targeting region complementary to one or more of the following prokaryotic and eukaryotic rRNA types: 5S, 16S, 23S, 5.8S, 17S, 18S, and/or 28S. A bridging nucleic acid may target 1, 2, 3, 4, 5, 6, 7, or more types of rRNA, as well as any and all tRNA types, both eukaryotic and prokaryotic.

A “bridging region” in a bridging nucleic acid refers to a region that mediates an interaction with a capture nucleic acid. In further embodiments, the bridging region is a polypurine or polypyrimidine stretch of residues. A bridging region can include a stretch of adenine or guanine residues or cytosine, uracil, or thymidine residues. In some embodiments, it is contemplated that more than one bridging region is included in a bridging nucleic acid, such as 2, 3, 4, 5, or more bridging regions.

A “capture nucleic acid” refers to a molecule that includes nucleotides or nucleotide analogs, a capture region, and a nonreacting structure. A “capture region” refers to a region that interacts with the bridging region of a bridging nucleic acid. In embodiments of the invention, the bridging region and the capture region are complementary to each other and hybridize to one another under conditions that allow for hybridization of complementary regions. There may be more than one nonreacting structure attached, covalently or noncovalently, to a capture nucleic acid. There may be 1, 2, 3, 4, 5, 6, 7, 8, 9, 10 or more nonreacting structures as part of a capture nucleic acid.

A capture nucleic acid also includes a “nonreacting structure,” which refers to a compound that does not chemically react with a nucleic acid. In some embodiments, a nonreacting structure is a magnetic bead or rod, which allows the capture nucleic acid, a bridging nucleic acid and a target nucleic acid to be isolated from a sample with a magnetic field, such as a magnetic stand. In still further embodiments, the nonreacting structure is a bead or other structure that can be physically captured, such as by using a basket, filter, or by centrifugation. It is contemplated that a bead may include plastic, glass, teflon, silica, a magnet or be magnetizeable, a metal such as a ferrous metal or gold, carbon, cellulose, latex, polystyrene, and other synthetic polymers, nylon, cellulose, nitrocellulose, polymethacrylate, polyvinylchloride, styrene-divinylbenzene, or any chemically-modified plastic or any other nonreacting structure. In still further embodiments, the nonreacting structure is biotin or iminobiotin. Biotin or iminobiotin binds to avidin or streptavidin, which can be used to isolate the capture nucleic acid and any hybridizing molecules. Furthermore, in some embodiments of the invention, the nonreacting structure is cellulose or an analog thereof.

It is contemplated that the location of the targeting and bridging regions in the bridging nucleic acid may be at a variety of positions. The location of targeted regions in a targeted nucleic acid or a capture region in a capture nucleic acid may also vary. The location of any of these regions or nonreacting structure may be or be within 10, 20, 30, 40, 50, 60, 70, 80, 90, 100, 110, 12, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 or more nucleotides from the 3′ and/or 5′ end of the relevant nucleic acid (“relevant nucleic acid” refers to the nucleic acid in which the region is located). Moreover, it is contemplated that a region, such as a bridging, capture, targeted, or targeting region-as well as a nonreacting structure-may be at or within 100-5000 residues, 150-4000 residues, 200-3000 residues, 250-2000 residues, 300-1500 residues, 350-1000 residues, 400-900 residues, 450-800 residues, or 500-700 residues of the 5′ or 3′ end of the relevant nucleic acid.

Furthermore, it is also contemplated that the spacing between regions may vary. Regions in the same nucleic acid or a region and a nonreacting structure may be adjacent to one another or there may be residues between them or between each of them. The number of intervening residues may be the following or may be at least or at most of the following: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, or more nucleotides between them or each of them.

As for the location of the sequence to which the targeting region is complementary, termed “targeted region,” this may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 nucleotides or more from the 3′ and/or 5′ end of the targeted nucleic acid. It is specifically contemplated that the targeting region hybridizes to a sequence located between 100 and 5000, 150 and 4000, 200 and 3000, 250 and 2000, and 300 and 1000 residues of the 5′ and/or 3′ end of the targeted nucleic acid. It is also contemplated that the targeted region is at the 3′ or 5′ end of the targeted nucleic acid. Alternatively, the targeted region may not be within 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 or more nucleotides from the termini of a targeted nucleic acid.

In some embodiments, the targeting region comprises or is complementary to all or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 or more contiguous nucleotides of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:71, SEQ ID NO:72, or SEQ ID NO:73 (collectively referred to as “SEQ ID NOS:1-73”). It is specifically contemplated that targeting regions of the invention comprise, in some embodiments, at least 5 contiguous nucleotides of SEQ ID NO:1-22; it is also contemplated that targeting regions of the invention are complementary to a sequence (“sequence” in the context of complementary regions refers to a sequence of at least 5, 6, 7, 8, 9, 10, 15, 20, 25, 30, or more nucleotides in length) of SEQ ID NOS:23-73, which are sequences of rRNA molecules.

It will be understood that any embodiment discussed with respect to nucleotides applies also when nucleotide analogs are used. It is specifically contemplated that nucleotide analogs may be employed with respect to bridging and capture nucleic acids of the invention.

It is contemplated that nucleic acids of the invention include RNA, DNA, locked nucleic acid™ (LNA), iso-bases, and/or peptide mimetics. It is contemplated that all or part of nucleic acids of the invention may include such nucleic acid components.

The present invention further concerns methods of isolating and/or depleting nucleic acids from a sample. In some embodiments, methods include a) incubating a sample with a first bridging nucleic acid comprising (1) at least one bridging region comprising at least 5 nucleic acid residues, under conditions allowing hybridization between the first targeting region and the targeted nucleic acid; b) incubating the first bridging nucleic acid with a capture nucleic acid comprising a nonreacting structure and a capture region comprising at least 5 nucleic acid residues, under conditions that allowing hybridization between the first bridging region and the capture region. In additional embodiments, one or more other steps may be included in combination with the method discussed above. Other steps involve isolating the targeted nucleic acid from the remainder of the sample; discarding the portion of the sample that hybridizes directly or indirectly to the capture nucleic acid (indirect hybridization refers to specific association of compounds that occurs through hybridization with a mediating compound, for example, indirect hybridization of a capture nucleic acid and a targeted nucleic acid via hybridization to a bridging nucleic acid); incubating the sample with additional bridging nucleic acids, under conditions allowing hybridization between the targeting region of the additional bridging nucleic acid and the targeted nucleic acid; implementing the method with respect to other targeted nucleic acids; washing the capture nucleic acid after incubation with the sample and the bridging nucleic acid; incubating the capture nucleic acid, bridging nucleic acid, and sample with elution buffer after isolating the targeted nucleic acid from the rest of the sample; eluting the targeted nucleic acid from the nonreactant structure; using the capture nucleic acid in a subsequent method involving a new sample; discarding the targeted nucleic acid after separating it from the sample; performing hybridizations between the bridging nucleic acid and the sample and the capture nucleic acid and the sample at the same temperatures or at different temperatures; performing the above hybridization steps at the same time, sequentially (one after the other or the other after the one); exposing the sample to a magnetic field or magnet, particularly when a magnetic bead or other object comprises all or part of the nonreacting structure of the capture nucleic acid; and incubating the sample with streptavidin or avidin, particularly if biotin or iminobiotin is used as a non-reacting structure.

In some embodiments of the invention, the sample, a bridging nucleic acid and/or a capture nucleic acid are incubated in a buffer, which, in some embodiments, includes TEAC or TMAC.

In methods of the invention involving more than one bridging nucleic acid, it is contemplated that the targeting region of the first bridging nucleic acid may be complementary to a different sequence of a different targeted nucleic acid than a targeting region of another bridging nucleic acid. Alternatively, different bridging nucleic acids may have targeting regions that are complementary to the same targeted nucleic acid. In the latter case, it is further contemplated that the targeting regions be complementary to sequences that overlap one another or ma be complementary to sequences in non-overlapping locations.

In cases in which targeting regions are complementary to different targeted nucleic acids, embodiments may involve targeting the largest rRNA molecule in a sample with one bridging nucleic acid and the second largest rRNA molecule in a sample with another bridging nucleic acid. In still further embodiments, another or third bridging nucleic acid will target the third largest rRNA molecule in a sample, while another or a fourth bridging nucleic acid will target the fourth largest rRNA molecule in a sample.

In another embodiment of the invention, there is a method for depleting rRNA from a sample comprising incubating the sample with (1) at least a first bridging oligonucleotide comprising a bridging region comprising a polypurine region of at least 5 residues in length and a targeting region comprising at least 5 contiguous residues complementary to an rRNA molecule in the sample and (2) a capture oligonucleotide comprising a magnetic bead and a capture region comprising a polypyrimidine region of at least 5 residues in length, under conditions allowing hybridization between the bridging oligonucleotide and the capture oligonucleotide and between the bridging oligonucleotide and the rRNA; b) incubating the sample with a magnetic bead; and c) isolating the magnetic bead. In still further embodiments, the first bridging oligonucleotide comprises a targeting region complementary to prokaryotic 23S rRNA. In still further embodiments, there is a second bridging oligonucleotide with a targeting region complementary to a different region of a prokaryotic 23S RNA than the first bridging oligonucleotide. In even further embodiments, there is a third and fourth bridging oligonucleotide each with a targeting region complementary to different sequences of a prokaryotic 16S rRNA.

As discussed earlier, a sample may be depleted or isolated as a way of enriching for the nontargeted nucleic acid, such as mRNA. In further embodiments of the invention, enriched mRNA can be used to prepare cDNA according to methods known to those of ordinary skill in the art, and as described herein. Thus, in cases in which mRNA is enriched as a result of methods of the invention, embodiments may further include discarding the portion of the sample that hybridizes to the capture oligonucleotide. More specifically targeted rRNA may be discarded and the mRNA remaining in the sample may be used to produce cDNA molecules. cDNA molecules may be used in a variety of methods, including, but not limited to, library production, production of proteins, and for creating and screening arrays. Therefore, in some embodiments of the invention, cDNA made from mRNA enriched according to methods of the invention are attached to a solid support or surface so as to create a nucleic acid array. The term “nucleic acid array” refers to a plurality of target elements, wherein each target element comprising one or more nucleic acid molecules immobilized on one or more solid surfaces at discrete locations to which sample nucleic acids can by hybridized. The nonreacting solid surface or support may be any of a number of materials, including plastic, glass, or nylon. In some embodiments, the solid support is a plate. The plate may have wells that contain the target elements. Plates may have 2, 3, 4, 5, 6, 7, 8, 9, 10 or more wells (“multi-well”), and up to at least 96 or 192 wells. In some embodiments of the invention, the sample nucleic acids comprise cDNAs made by depleting a sample of rRNA, according to methods of the invention. Those embodiments may further involve contacting a nucleic acid array with the cDNA. Alternatively, cDNA made according to the invention may be used as target elements on an array.

The present invention also concerns kits that include compositions of the invention to implement the methods discussed herein. These kits can be used for the depletion, isolation, or purification of nucleic acids. Kits contain these compositions in a suitable container means.

In some embodiments, a kit includes 1) at least one capture oligonucleotide comprising a capture region and a magnetic bead; and 2) at least a first bridging oligonucleotide comprising i) at least one bridging region complementary to all or part of the capture region of the capture oligonucleotide and ii) at least one targeting region comprising 10 contiguous nucleic acids complementary to an rRNA.

In additional embodiments, there is a second bridging oligonucleotide comprising i) at least one bridging region complementary to all or part of the capture region of the capture oligonucleotide and ii) at least one targeting region comprising 10 contiguous nucleic acids complementary to an rRNA. In some kits, the targeting region of the second bridging oligonucleotide is complementary to the same rRNA as the targeting region of the first bridging oligonucleotide, while in other embodiments, these are complementary to different rRNAs. Further embodiments involve kits in which the targeting region of the first bridging oligonucleotide is complementary to the largest rRNA of a prokaryote or eukaryote. In other embodiments, the second bridging oligonucleotide has a targeting region that is complementary to either the largest rRNA of a prokaryote or eukaryote or the second largest rRNA of a prokaryote or eukaryote. It is specifically contemplated that kits may include one or more bridging oligonucleotides targeting prokaryotic rRNA (16S, 23S, or both) and one or more bridging oligonucleotides targeting eukaryotic rRNA (18S, 28S, or both); thus, a kit may be used for depleting both eukaryotic and prokaryotic rRNA, in some embodiments.

Kits may also include a third, fourth, fifth, sixth, seventh, eighth, ninth, tenth or more bridging oligonucleotides with targeting region complementary to the same or different rRNAs as the targeting regions of the first and second bridging oligonucleotides. It is contemplated that the targeting regions of the bridging oligonucleotides in kits of the invention may be complementary to prokaryote 16S rRNA, prokaryote 23S rRNA, prokaryote 5S rRNA, eukaryote 17S or 18S rRNA, eukaryote 28SrRNA, and/or eukaryote 5.8S rRNA. It is further contemplated that targeting regions of bridging oligonucleotides in kits may have all or part of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, or SEQ ID NO:22 (collectively referred to as “SEQ ID NOS:1-22”). Alternatively, kits may include targeting regions as discussed above with respect to SEQ ID NOS:23-73, i.e. targeting regions complementary to a sequence from SEQ ID NOS:23-73. Kits of the invention may also include one or more of the following: binding buffer with TMAC, binding buffer with TEAC, magnetic stand, wash solution, nuclease-free water; RNAse inhibitors, glycogen, control RNA, sodium acetate, ammonium acetate, streptavidin beads, avidin beads, magnetic beads, beads of any nonreacting structure—including those discussed above—capture basket; capture filters, RNA markers, nuclease-free containers such as tubes and tips, and any other composition described herein.

It is contemplated that kits of the invention may be used to implement methods of the invention, that methods of the invention may be implemented with compositions of the invention, and that kits may include any composition of the invention.

It is further contemplated that kits, methods, and compositions of the invention may effect a depletion of a targeted nucleic acid in a sample by reducing its amount in the sample by at least 10, 15, 20, 25, 30, 35, 40, 45, 50, 55, 60, 65, 70, 75, 80, 85, 90, 95, 96, 97, 98, 99, 99.1, 99.2, 99.3, 99.4, 99.5, 99.6, 99.7, 99.8, 99.9 or more percent.

Kits of the invention also include materials for creating a nucleic acid array. Any of the kits discussed above may also include a solid support for preparing a nucleic acid array.

The use of the word “a” or “an” when used in conjunction with the term “comprising” in the claims and/or the specification may mean “one,” but it is also consistent with the meaning of “one or more,” “at least one,” and “one or more than one.” When the term “at least” is used in the context of bridging, targeting, or capture regions, as well as for capture and bridging oligonucleotides, it is contemplated that there is an upper limit of 20 for practical purposes, even though more such regions or oligonucleotides could be implemented with the invention. Furthermore, it should be understood that a number (cardinal or ordinal) used in the context of compositions of the invention refers to a “kind” of that composition; thus, “a first oligonucleotide” in the context of a “second oligonucleotide” refers to “one of that kind of oligonucleotide,” and not one single oligonucleotide molecule.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Depiction of molecules in system. A bridging oligonucleotide is shown with a targeting region and a bridging region. The targeting region is complementary to a targeted region in the targeted nucleic acid, which is an rRNA molecule. The bridging region is complementary to the capture region in the capture oligonucleotide, which is attached, by way of example, to a magnetic bead as a nonreacting structure.

FIGS. 2A-1 to A-14 and FIGS. 2B-1 to B-27. Sequence comparison of different rRNAs from different bacteria to E. coli rRNA with MegAlign sequence analysis software version 4.05 from DNA Star, Incorporated. A. The 5′ end of the sequence is shown on the first page of the figure in FIG. 2A-1 and continues until the last page of the figure, FIG. 2A-14, in which the 3′ end of the same sequence is shown. Shown is a sequence comparison of 16S rRNA of listed prokaryotic organisms to 16S rRNA from E. coli (SEQ ID NO:34). The sequences are the 16S rRNA from the following organisms: B. subtilis (SEQ ID NO:23); B. anthracis (SEQ ID NO. 24); E. faecalis (SEQ ID NO. 25); L. lactis (SEQ ID NO. 26); L. monocyt (SEQ ID NO. 27); S. aureus (SEQ ID NO. 28); S. mutans (SEQ ID NO. 29); S. pneumon (SEQ ID NO. 30); S. pyogenes (SEQ ID NO. 31); M. avian (SEQ ID NO. 32); M. tuberculosis (SEQ ID NO. 33); K. pneumoniae (SEQ ID NO. 35); A. actino (SEQ ID NO. 36); H. influenzae (SEQ ID NO. 37); E. bronchiseptica (SEQ ID NO. 38); B. parapertussis (SEQ ID NO. 39); B. pertussis (SEQ ID NO. 40); B. cepacia (SEQ ID NO. 41); B. mallei (SEQ ID NO. 42); B. pseudomallei (SEQ ID NO. 43); N. gonorrhoeae (SEQ ID NO. 44); N. mening (SEQ ID NO. 45); P. aeruginosa (SEQ ID NO. 46); V. cholerae (SEQ ID NO. 47); and Y. enterocolitica (SEQ ID NO. 48). B. The 5′ end of the sequence is shown on the first page of the figure in FIG. 2B-1 and continues until the last page of the figure, FIG. 2B-27, in which the 3′ end of the same sequence is shown. Shown is a sequence comparison of 23S rRNA of listed prokaryotic organisms to 23S rRNA from E. coli (SEQ ID NO:60). The sequences are the 23S rRNA from the following organisms: B. subtilis (SEQ ID NO:49); B. anthracis (SEQ ID NO. 50); E. facaelis (SEQ ID NO. 51); L. lactis (SEQ ID NO. 52); L. monocytogenes (SEQ ID NO. 53); S. aureus (SEQ ID NO. 54); S. mutans (SEQ ID NO. 55); S. pneumoniae (SEQ ID NO. 56); S. pyogenes (SEQ ID NO. 57); M. avium (SEQ ID NO. 58); M. tuberculosis (SEQ ID NO. 59); K. pneumoniae (SEQ ID NO. 61); H. influenzae (SEQ ID NO. 62); B. bronchiseptica (SEQ ID NO. 63); B. parapertussis (SEQ ID NO. 64); B. pertussis (SEQ ID NO. 65); B. cepacia (SEQ ID NO. 66); E. mallei (SEQ ID NO. 67); E. pseudomallei (SEQ ID NO. 68); N. gonorrhoeae (SEQ ID NO. 69); N. eminigititdis (SEQ ID NO. 70); P. aeruginosa (SEQ ID NO. 71); V. cholerae (SEQ ID NO. 72); Y. enterocolitica (SEQ ID NO. 73).

FIG. 3. Electropherograms of RNA from a control reaction. E. coli total RNA was purified with RNAwiz™ (Ambion) and carried through the rRNA depletion procedure as described in Example 2, except that bridging nucleic acids were left out of the reaction. A sample of the RNA was analyzed with the RNA 6000 Lab Chip Kit® (Caliper Technologies Corp.) using the Agilent 2100 Bioanalyzer (Agilent Technologies). The electropherogram shown was generated with Agilent 2100 Bioanalyzer Bio Sizing software (Version A.02.01).

FIG. 4. Electropherograms of RNA from an experimental reaction after ribosomal RNA depletion. E. coli total RNA was purified with RNAwiz™ (Ambion) and carried through the rRNA depletion procedure as described in Example 2. A sample of the RNA was analyzed as described in the legend to FIG. 3.

FIGS. 5A-B. Electropherograms of RNA from experiments. A. Agilent 2100 Bioanalyzer electropherogram of a sample from a control reaction performed as described in Example 5, but with no bridging oligonucleotides. The sample contains E. coli and rat liver total RNA. The RNA sample was analyzed with the RNA 6000 Lab Chip Kit® (Caliper Technologies Corp.) using the Agilent 2100 Bioanalyzer (Agilent Technologies). The electropherogram shown was generated with Agilent 2100 Bioanalyzer Bio Sizing software (Version A.02.01). B. Agilent 2100 Bioanalyzer electropherogram of a sample from an experimental reaction performed as described in Example 5 with bridging oligonucleotides. The sample is depleted of E. coli 16S and 23S rRNA and rat liver 18S and 28S rRNA. The RNA sample was analyzed with the RNA 6000 Lab Chip Kit® (Caliper Technologies Corp.) using the Agilent 2100 Bioanalyzer (Agilent Technologies). The electropherogram shown was generated with Agilent 2100 Bioanalyzer Bio Sizing software (Version A.02.01).

FIGS. 6A-B. Electropherograms of RNA from experiments. A. Agilent 2100 Bioanalyzer electropherogram of a sample from a control reaction performed as described in Example 6, but with no bridging oligonucleotides. The sample contains human liver total RNA. The RNA sample was analyzed with the RNA 6000 Lab Chip Kit® (Caliper Technologies Corp.) using the Agilent 2100 Bioanalyzer (Agilent Technologies). The electropherogram shown was generated with Agilent 2100 Bioanalyzer Bio Sizing software (Version A.02.01). B. Agilent 2100 Bioanalyzer electropherogram of a sample from an experimental reaction performed as described in Example 6 with bridging oligonucleotides. The sample is depleted of human 18S and 28S rRNA. The RNA sample was analyzed with the RNA 6000 Lab Chip Kit® (Caliper Technologies Corp.) using the Agilent 2100 Bioanalyzer (Agilent Technologies). The electropherogram shown was generated with Agilent 2100 Bioanalyzer Bio Sizing software (Version A.02.01).

FIGS. 7A-B. Electropherograms of RNA from experiments. A. Agilent 2100 Bioanalyzer electropherogram of a sample from a control reaction performed as described in Example 7, but with no bridging oligonucleotides. The sample contains rat liver total RNA. The RNA sample was analyzed with the RNA 6000 Lab Chip Kit® (Caliper Technologies Corp.) using the Agilent 2100 Bioanalyzer (Agilent Technologies). The electropherogram shown was generated with Agilent 2100 Bioanalyzer Bio Sizing software (Version A.02.01). B. Agilent 2100 Bioanalyzer electropherogram of a sample from an experimental reaction performed as described in Example 6 with bridging oligonucleotides. The sample is depleted of rat 18S and 28S rRNA. The RNA sample was analyzed with the RNA 6000 Lab Chip Kit® (Caliper Technologies Corp.) using the Agilent 2100 Bioanalyzer (Agilent Technologies). The electropherogram shown was generated with Agilent 2100 Bioanalyzer Bio Sizing software (Version A.02.01).

FIGS. 8A-B. Electropherograms of RNA from experiments. A. Agilent 2100 Bioanalyzer electropherogram of a sample from a control reaction performed as described in Example 6, but with no bridging oligonucleotides. The sample contains mouse liver total RNA. The RNA sample was analyzed with the RNA 6000 Lab Chip Kit® (Caliper Technologies Corp.) using the Agilent 2100 Bioanalyzer (Agilent Technologies). The electropherogram shown was generated with Agilent 2100 Bioanalyzer Bio Sizing software (Version A.02.01). B. Agilent 2100 Bioanalyzer electropherogram of a sample from an experimental reaction performed as described in Example 8 with bridging oligonucleotides. The sample is depleted of mouse 18S and 28S rRNA. The RNA sample was analyzed with the RNA 6000 Lab Chip Kite (Caliper Technologies Corp.) using the Agilent 2100 Bioanalyzer (Agilent Technologies). The electropherogram shown was generated with Agilent 2100 Bioanalyzer Bio Sizing software (Version A.02.01).

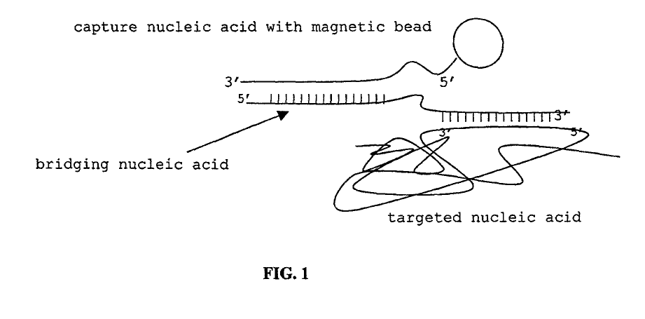

### DESCRIPTION OF ILLUSTRATIVE EMBODIMENTS

The present invention concerns a system for isolating, depleting, or identifying specific, targeted nucleic acid populations, such as rRNA in a sample, in some cases for the purpose of enriching for other nucleic acid populations. The targeted nucleic acid, components of the system, and the methods for implementing the system, as well as variations thereof, are provided below.

I. Targeted Nucleic Acid

The present invention concerns targeting a particular nucleic acid population (i.e., mRNA, rRNA, tRNA, genomic DNA) or targeting types of a nucleic acid population, such as individual tRNAs, rRNAs (5S, 16S, or 23S rRNA from prokaryotes; 5.8S, 17S or 18S, or 28S from eukaryotes), or specific mRNAs. A nucleic acid is targeted by using a bridging nucleic acid that has a targeting region—a region complementary to all or part of the targeted nucleic acid.

In some embodiments, the invention is specifically concerned with depleting or isolating rRNA from other nucleic acids (“non-targeted nucleic acids” or “enriched population”). The 5S, 16S, and/or 23S rRNA from a prokaryote may be the targeted nucleic acid. Also, the 5.8S, 17S (observed in yeast) or 18S, and/or 28S from a eukaryote may be the targeted nucleic acid. Alternatively, rRNAs from both prokaryotes and eukaryotes may be targeted, such as with a sample that has eukaryotic host cells infected with a prokaryotic organism. The sequences for ribosomal RNAs are well known to those or ordinary skill in the art and can be readily found in sequence databases such as GenBank (www.ncbi.nlm.nih.gov/) or are published. Nucleic acids may be targeted by targeting regions that are complementary to all or part of the targeted nucleic acid. Targeted nucleic acids may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, or more nucleotides in length. Furthermore, any region of at least five contiguous nucleotides in the targeted nucleic acid may be used as the targeted region-that is, the region that is complementary to the targeting region of a bridging nucleic acid. Also, there may be more than one targeted region in a targeted nucleic acid. There may be, be at least, or be at most 1, 2, 3, 4, 5, 6, 7, 8, 9, 10 or more targeted regions in a targeted nucleic acid. A targeted region may be a region in a targeted nucleic acid that has greater than 70%, 80%, or 90% homology with a sequence from a different targeted nucleic acid. In some embodiments, the targeted region from a targeted nucleic acid is identical to a sequence in a different targeted nucleic acid. For example, 23S rRNA of various prokaryotes may be targeted using a targeted region common to a group of organisms, such as Gram negative bacteria or a subset of such bacteria. Alternatively, a targeted region may be a sequence unique to a particular targeted nucleic acid. However, for purposes of this application, a “targeted region” is not a poly-A region, such as a poly-A tail of an eukaryotic mRNA. Additional information regarding targeted rRNAs is provided below. This information is provided as an example of targeted nucleic acids. However, it is contemplated that there may be sequence variations from individual organism to organism and these sequences provided as simply an example of one sequenced nucleic acid, even though such variations exist in nature. It is contemplated that these variations may also be targeted, and this may or may not require changes to a targeting nucleic acid or to the hybridization conditions, depending on the variation, which one of ordinary skill in the art could evaluate and determine.

A number of patents concern a targeted nucleic acid, for example, U.S. Pat. Nos. 4,486,539; 4,563,419; 4,751,177; 4,868,105; 5,200,314; 5,273,882; 5,288,609; 5,457,025; 5,500,356; 5,589,335; 5,702,896; 5,714,324; 5,723,597; 5,759,777; 5,897,783; 6,013,440; 6,060,246; 6,090,548; 6,110,678; 6,203,978; 6,221,581; 6,228,580; and WO 01/32672, all of which are specifically incorporated herein by reference.

A. Prokaryotic rRNA

Prokaryotic rRNA can be a targeted nucleic acid of the invention. The following examples are provided, but the invention is not limited solely to these organisms and sequences (GenBank accession number provided and/or region within sequence that corresponds to the targeted rRNA):

1. Superkingdom Archaea (archaebacteria)

Aeropyrum pernix

| | |
|---|---|
| 16S | D83259 |

Aeropyrum pernix NC_000854

| | |
|---|---|
| APErRNA05 (16S) | 1218763-1220185 |
| APErRNA03 (23S) | 1213627-1218039 |

Methanococcus jannaschii

| | |
|---|---|
| 16S | M59126 |

Methanococcus jannaschii NC_000909

| | |
|---|---|
| MJrrnA16S | 157985-159459 |
| MJrrnA23S | 154759-157648 |

Halobacterium marismortui

| | |
|---|---|
| 23S | X13738 |

Halobacterium sp. NRC-1 NC_002607

| | |
|---|---|
| rrs (16S) | 1875505-1876977 |
| rr1A (23S) | 1877506-1880411 |

Thermoplasma acidophilum

| | |
|---|---|
| 23S | M32298 |

Thermoplasma acidophilum NC_002578

| | |
|---|---|
| 16S | 1475300-1475770 |

2. Superkingdom Eubacteria (eubacteria)

a. Firmicutes (Gram-positive bacteria)

| | |
|---|---|
| i) | Bacillus/Clostridium group (low G + C gram- |
| | positive bacteria) |
| | Listeria innocua Clip 11262 NC_003212 |

| | |
|---|---|
| 16S | 260527-262081 |
| 23S | 262327-265257 |

Listeria monocytogenes strain EGD NC_003210

| | |
|---|---|
| 16S | 237466-239020 |
| 23S | 239265-242195 |

Bacillus subtilis NC000964

| | |
|---|---|
| RrnO 16S | 9809-11361 |
| RrnA 23S | 11707-14634 |

Bacillus anthracis

| | |
|---|---|
| 16S (1508nt) | AF155950 |
| 23S (2922nt) | AF267877 |

Bacillus thuringiensis

| | |
|---|---|
| 16S (1486nt) | D16281 |
| 23S (2923nt) | AF267880 |

Staphylococcus aureus strain Mu50 NC_002758

| | |
|---|---|
| 16S | 530479-532033 |
| 23S | 532398-535231 |

Staphylococcus aureus N315 NC_002745

| | |
|---|---|
| SarRNA01 16S | 506138-507692 |
| SarRNA02 23S | 508166-510999 |

Clostridium acetobutylicum ATCC824 NC_003030

| | |
|---|---|
| 16SarRNA | 9710-11219 |
| 23SarRNA | 11398-14303 |

Clostridium difficile

| | |
|---|---|
| 16S (1470nt) | X73450 |

Clostridium perfringens

| | |
|---|---|
| 16S | M69264 (499-2294) |

Mycoplasma genitalium G37 L43967

| | |
|---|---|
| MgrrnA16S | 170009-171527 |
| MgrrnA23S | 171730-174463 |

Mycoplasma pneumoniae NC_000912

| | |
|---|---|
| 16S | 118312-119824 |
| 23S | 120057-122961 |

Mycoplasma pulmonis NC_002771

| | |
|---|---|
| 16S | 813583-815113 |
| 23S | 810563-813297 |

Streptococcus pneumoniae R6 NC_003098

| | |
|---|---|
| RRNA16S-1 | 15161-16674 |
| RRNA23S-1 | 16945-19846 |

Streptococcus pneumoniae TIGR4 AE005672

| | |
|---|---|
| SprrnaA16S | 15394-16806 |
| SprrnaA23S | 17142-20043 |

Streptococcus pyogenes AE004092

| | |
|---|---|
| 16S | 17170-18504 |
| 23S | 19037-21937 |

Streptococcus mutans

| | |
|---|---|
| 16S (1334nt) | X58303 |
| 23S | AF139599 (1940-4840) |

Lactococcus lactis

| | |
|---|---|
| 16S | X64887 (508-2055) |
| 23S | X64887 (2360-5257) |

Enterococcus faecalis

| | |
|---|---|
| 16S (1449nt) | Y18293 |
| 23S (2912nt) | AJ295306 |

| | |
|---|---|
| ii) | Actinobacteria (high G + C gram-positive |
| | bacteria) |
| | Mycobacterium leprae strain TN NC_002677 |

| | |
|---|---|
| Rrs16S | 1341144-1342692 |
| Rr123S | 1342976-1346100 |

Mycobacterium tuberculosis CDC 1551 NC_002755

| | |
|---|---|
| MtrrnaA16S | 1471388-1472923 |
| MtrrnaA23S | 1473199-1476336 |

Mycobacterium avium

| | |
|---|---|
| 16S (1372nt) | M61673 |
| 23S | X74494 (295-3401) |

Corynebacterium glutamicum

| | |
|---|---|
| 16S (1479nt) | Z46753 |

Rhodococcus equi

| | |
|---|---|
| 16S (1478nt) | X80614 |

| | |
|---|---|
| b. | Spirochaetales (spirochetes) |
| | Borrelia burgdorferi AE000783 |

| | |
|---|---|
| Rr1B 16S | 444581-446118 |
| Rr1B 23S | 438590-441508 |

Treponema pallidum AE000520

| | |
|---|---|
| TprrnaA16S | 230162-231656 |
| TprrnaA23S | 231950-234850 |

Borrelia burgdorferi

| | |
|---|---|
| 16S | AE001147 (9459-10996) |
| 23S | AE001147 (212-3145) |

| | |
|---|---|
| c. | Thermotogales |
| | Thermotoga maritima AE000512 |

| | |
|---|---|
| TmrrnaA16S | 188968-190526 |
| TmrrnaA23S | 190766-193787 |

| | |
|---|---|
| d. | Thermus/Deinococcus group |
| | Deinococcus radiodurans R1 NC_001263 |

| | |
|---|---|
| DrrrnaA16S | 2285518-2287019 |
| DrrrnaA23S | 2245319-2246194 |

Deinococcus radiodurans

| | |
|---|---|
| 16S | AE002076 (7275-8776) |
| 23S | AE001886 (8829-10771) |

| | |
|---|---|
| e. | Chlamydiales (chlamydias) |
| | Chlamydia trachomatis AE001273 |

| | |
|---|---|
| 16SrRNA1 | 854128-855677 |
| 23SrRNA1 | 855993-858862 |

Chlamydophila pneumoniae AR39 NC_002179

| | |
|---|---|
| CprrnA16S | 1069329-1070785 |
| CprrnA23S | 1066159-1069022 |

Chlamydophila psittaci

| | |
|---|---|
| 16S | U68447 (1-1553) |
| 23S | U68447 (1778-4721) |

| | |
|---|---|
| f. | Proteobacteria (purple bacteria) |

| | |
|---|---|
| i) | Alpha subdivision |
| | Rickettsia conorii Malish 7 NC_003103 |

| | |
|---|---|
| Rrs16S | 884601-886108 |
| Rr123S | 281797-284557 |

Rickettsia prowazekii strain Madrid E AJ235269

| | |
|---|---|
| Rrs16S | 772263-773769 |
| Rr123S | 257853-260613 |

Rickettsia typhi

| | |
|---|---|
| 16S (1444nt) | M20499 |
| 23S | Y13133 (956-3716) |

Ehrlichia bovis

| | |
|---|---|
| 16S (1488nt) | U03775 |

Agrobacterium tumefaciens C58 AE007870

| | |
|---|---|
| 16S | 768991-770427 |
| 23S | 765313-767565 |

Brucella melitensis

| | |
|---|---|
| 16S | AF220148 (645-2129) |
| 23S | AF220148 (2896-3024 . . . 3204-5807) |

Rhizobium rhizogenes

| | |
|---|---|
| 16S (1369nt) | D13945 |

| | |
|---|---|
| ii) | Beta subdivision |
| | Neisseria meningitides strain MC58 AE002098 |

| | |
|---|---|
| NmrrnaA16S | 60971-62514 |
| NmrrnaA23S | 63178-66068 |

Bordetella bronchiseptica

| | |
|---|---|
| 16S (1532nt) | X57026 |
| 23S (2865nt) | X70371 |

Bordetella parapertussis

| | |
|---|---|
| 16S (1464nt) | U04949 |
| 23S (2865nt) | X68368 |

Bordetella pertussis

| | |
|---|---|
| 16S (1464nt) | U04950 |

Burkholderi mallei

| | |
|---|---|
| 16S (1488nt) | AF110188 |
| 23S (2882nt) | Y17183 |

Burkholderi pseudomallei

| | |
|---|---|
| 16S (1488nt) | U91839 |
| 23S (2882nt) | Y17184 |

Neisseria gonorrhoeae

| | |
|---|---|
| 16S (1544nt) | X07714 |
| 23S (2890nt) | X67293 |

| | |
|---|---|
| iii) | Gamma group |
| | Buchnera sp. APS NC_002528 |

| | |
|---|---|
| Rrs 16S | 274065-275524 |
| Rr1 23S | 539539-542451 |

Escherichia coli K12 U00096

| | |
|---|---|
| RrsH 16S | 223771-225312 |
| Rr1H 23S | 225759-228662 |

Escherichia coli 0157:H7 NC_002695

| | |
|---|---|
| RrsH 16S | 227102-228643 |
| Rr1H 23S | 229090-231992 |

Salmonella enterica serovar Typhi NC_003198

| | |
|---|---|
| 16S | 287479-289020 |
| 23S | 289375-292380 |

Salmonella typhimurium LT2 NC_003197

| | |
|---|---|
| RrsH 16S | 289189-290732 |
| RrlH 23S | 291244-294336 |

Yersinia pestis NC_003143

| | |
|---|---|
| 16S | 12292-13763 |
| 23S | 14272-17178 |

Klebsiella pneumoniae

| | |
|---|---|
| 16S (1534nt) | X87276 |
| 23S (2903nt) | X87284 |

Yersinia enterocolitica

| | |
|---|---|
| 16S (1484nt) | Z49830 |
| 23S (2906nt) | U77925 |

Proteus vulgaris

| | |
|---|---|
| 16S (2067nt) | X07652 |

Shigella flexneri

| | |
|---|---|
| 16S (1468nt) | X80679 |

Shigella sonnei

| | |
|---|---|
| 16S (1467nt) | X80726 |

Shigella dysenterica

| | |
|---|---|
| 16S (1487nt) | X96966 |

Haemophilus influenzae Rd L42023

| | |
|---|---|
| HirrnE16S | 1511137-1512634 |
| HirrnE23S | 123801-126697 |

Pasteurella multocida

| | |
|---|---|
| 16S (1543nt) | M35018 |

Actinobacillus actinomycetemcomitans

| | |
|---|---|
| 16S (1485nt) | M75037 |

Actinobacillus pleuropneumoniae

| | |
|---|---|
| 16S | D30032 (83-1625) |

Haemophilus somnus

| | |
|---|---|
| 16S (1483nt) | M75046 |

Legionella pneumophila

| | |
|---|---|
| 16S (1544nt) | M59157 |

Mannheimia haemolytica

| | |
|---|---|
| 16S (1472nt) | U57072 |

Vibrio cholerae chromosomel NC_002505

| | |
|---|---|
| 16Sa rRNA | 53823-55357 |
| 23Sa rRNA | 55784-58670 |

Vibrio parahaemolyticus

| | |
|---|---|
| 16S (1499nt) | M59161 |

Coxiella burnetii

| | |
|---|---|
| 16S (1484nt) | M21291 |
| 23S | X79704 (1620-3350) |

Aeromonas hydrophila

| | |
|---|---|
| 16S (1538nt) | X87271 |

Aeromonas salmonicida

| | |
|---|---|
| 16S (1502nt) | X60405 |

Francisella tularesis

| | |
|---|---|
| 16S (1517nt) | Z21931 |

Moraxella catarrhalis

| | |
|---|---|
| 16S (1511nt) | U10876 |

Pseudomonas aeruginosa AE004091

| | |
|---|---|
| 16S | 722096-726631 |
| 23S | 724103-726993 |

Pseudomonas putida

| | |
|---|---|
| 16S (1527nt) | D84020 |

| | |
|---|---|
| iv) | Delta/Epsilon subdivisions |
| | Campylobacter jejuni AL111168 |

| | |
|---|---|
| 16S | 39249-40761 |
| 23S | 41568-44457 |

Helicobacter pylori 26695 NC_000915

| | |
|---|---|
| HPrrnB16S | 1511137-1512634 |
| HPrrnB23S | 1473918-1476893 |

| | |
|---|---|
| g. | Cyanobacteria |
| | Synechocystis sp. PCC 6803 NC_000911 |

| | |
|---|---|
| Rrn16Sa | 2452187-2453675 |
| Rrn23Sa | 2448839-2451721 |

Synechococcus sp. (Anacystis nidulans)

| | |
|---|---|
| 16S | X03538 (1432-2918) |
| 23S | X00512 (251-3126) |

| | |
|---|---|
| h. | CFB/Green sulfer bacteria group |
| | Porphyromonas gingivalis |

| | |
|---|---|
| 16S (1474nt) | L16492 |

B. Eukaryotic rRNA

Targeted nucleic acids of the invention may also be one or more types of eukaryotic rRNAs. Eukaryotes include, but are not limited to: mammals, fish, birds, amphibians, fungi, and plants. The following provides sequences for some of these targeted nucleic acids. It is contemplated that other eukaryotic rRNA sequences can be readily obtained by one of ordinary skill in the art, and thus, the invention includes, but is not limited to, the sequences shown below.

Superkingdom Eukaryota (eucaryotes)

Homo sapiens (human)

| | |
|---|---|
| 18S | M10098 |
| 18S | K03432 |
| 18S | X03205 |
| 28S | M11167 |

Mus muculus

| | |
|---|---|
| 18S | X00686 |
| 28S | X00525 |

Rattus norvegicus

| | |
|---|---|
| 18S | M11188 |
| 18S | X01117 |

Rattus norvegicus V01270.1

| | |
|---|---|
| 18S | 1-1874 |
| 28S | 3862-8647 |

II. Isolation and/or Depletion System Nucleic Acids

The present invention concerns compositions comprising a nucleic acid or a nucleic acid analog in a system or kit to deplete, isolate, or separate a nucleic acid population from other nucleic acid populations, for which enrichment may be desirable. It concerns a bridging nucleic acid and a capture nucleic acid to deplete, isolate, or separate out a targeted nucleic acid, as discussed above.

A. Bridging Nucleic Acids

Bridging nucleic acids of the invention comprise a bridging region and a targeting region. As discussed in other sections, the location of these regions may be throughout the molecule, which may be of a variety of lengths. The bridging nucleic acid may comprise RNA, DNA, both, or analogs of either or both.

The bridging region comprises a sequence that is complementary to at least five contiguous nucleotides in the capture nucleic acid. It is contemplated that that this region may be a homogenous sequence, that is, have the same nucleotide repeated across its length, such as a repeat of A, C, G, T, or U residues. However, to avoid hybridizing with a poly-A tailed mRNA in a sample comprising eukaryotic nucleic acids, it is contemplated that most embodiments will not have a poly-U or poly-T bridging region when dealing with such samples having poly-A tailed RNA. In some embodiments, the bridging region is a poly-C region and the capture region is a poly-G region, or vice versa. In other embodiments, the bridging region will be a random sequence that is complementary to the capture region (or the capture region will be random and the bridging region will be complementary to it). In further embodiments, the bridging region will have a designed sequence that is not homopolymeric but that is complementary to the capture region or vice versa. Sequences may be determined empirically. In many embodiments, it is preferred that this will be a random sequence or a defined sequence that is not a homopolymer. Some sequences will be determined empirically during evaluation in the assay.

B. Capture Nucleic Acids

Capture nucleic acids of the invention comprise a capture region and a nonreacting structure that allows the capture nucleic acid, any molecules specifically binding or hybridizing to the capture nucleic acid—such as the bridging nucleic acid—and any molecules specifically binding or hybridizing to the bridging nucleic acid—such as the targeted nucleic acid—to be isolated away from other nucleic acid populations.

The capture nucleic acid may comprise RNA, DNA, both, or analogs of either or both. However, in some embodiments of the invention, it is specifically contemplated to be homopolymeric (only one type of nucleotide residue in molecule, such as poly-C), though in other embodiments, it is specifically contemplated not to be homopolymeric and be heteropolymeric, as described for bridging regions.

1. Capture Regions

The main requirement for bridging and capture nucleic acid sequences is that they are complementary to one another. The capture region may be a poly-pyrimidine or poly-purine region comprising at least 5 nucleic acid residues. In addition, it may be heteropolymeric, either a random sequence or a designed sequence that is complementary to the bridging region of the nucleic acid with which it should hybridize.

2. Nonreacting Structures

A nonreacting structure is a compound or structure that will not react chemically with nucleic acids, and in some embodiments, with any molecule that may be in a sample. Nonreacting structures may comprise plastic, glass, teflon, silica, a magnet, a metal such as gold, carbon, cellulose, latex, polystyrene, and other synthetic polymers, nylon, cellulose, nitrocellulose, polymethacrylate, polyvinylchloride, styrene-divinylbenzene, or any chemically-modified plastic. They may also be porous or non-porous materials. The structure may also be a particle of any shape that allows the targeted nucleic acid to be isolated, depleted, or separated. It may be a sphere, such as a bead, or a rod, or a flat-shaped structure, such as a plate with wells. Also, it is contemplated that the structure may be isolated by physical means or electromagnetic means. For example, a magnetic field may be used to attract a non-reacting structure that includes a magnet. The magnetic field may be in a stand or it may simply be placed on the side of a tube with the sample and a capture nucleic acid that is magnetized. Examples of physical ways to separate nucleic acids with their specifically hybridizing compounds are well known to those of skill in the art. A basket or other filter means may be employed to separate the capture nucleic acid and its hybridizing compounds (direct and indirect). The non-reacting structure and sample with nucleic acids of the invention may be centrifuged, filtered, dialyzed, or captured (with a magnet). When the structure is centrifuged it may be pelleted or passed through a centrifugible filter apparatus. The structure may also be filtered, including filtration using a pressure-driven system. Many such structures are available commercially and may be utilized herewith. Other examples can be found in WO 86/05815, WO90/06045, U.S. Pat. No. 5,945,525, all of which are specifically incorporated by reference.

Cellulose is a structural polymer derived from vascular plants. Chemically, it is a linear polymer of the monosaccharide glucose, using β, 1-4 linkages. Cellulose can be provided commercially, including from the Whatman company, and can be chemically sheared or chemically modified to create preparations of a more fibrous or particulate nature. CF-1 cellulose from Whatman is an example that can be implemented in the present invention.

Synthetic plastic or glass beads may be employed in the context of the invention. The beads may be complexed with avidin or streptavidin and they may also be magnetized. The complexed streptavidin can be used to capture biotin linked to an oligo-dT or -U or poly (dT) or poly(U) moiety, either before or after hybridization to the poly(A) tails of mRNA. Alternatively, the oligo/poly(dT/U) moiety can be attached to the beads directly through chemical coupling. The beads may be collected using gravity- or pressure-based systems and/or filtration devices. If the beads are magnetized, a magnet can be used to separate the beads from the rest of the sample. The magnet may be employed with a stand or a stick or other type of physical structure to facilitate isolation.

Other components include isolation apparatuses such as filtration devices, including spin filters or spin columns.

C. Nucleic Acid Compositions

Embodiments of the present invention concern bridging, capture, and targeted nucleic acids. In particular aspects, a targeted nucleic acid encodes for or comprises a transcribed nucleic acid. In other aspects, a bridging nucleic acid comprises a targeting region that comprises a nucleic acid segment having the sequence of all or part of SEQ ID NO:1, SEQ ID NO:2, SEQ ID NO:3, SEQ ID NO:4, SEQ ID NO:5, SEQ ID NO:6, SEQ ID NO:7, SEQ ID NO: 8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:71, SEQ ID NO:72, or SEQ ID NO:73 (collectively referred to as “SEQ ID NOS:1-73”). In particular aspects, a targeted nucleic acid encodes a protein, polypeptide, peptide. Nucleic acids of the invention comprise RNA, DNA, analogs of RNA, analogs of DNA, or a combination thereof.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

1. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those of a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table of non-limiting, purine and pyrimidine derivatives and analogs is also provided herein below.

TABLE 1
Purine and Pyrimidine Derivatives or Analogs

| Abbr. | Modified base description |
|---|---|
| ac4c | 4-acetylcytidine |
| Chm5u | 5-(carboxyhydroxylmethyl) uridine |
| Cm | 2′-O-methylcytidine |
| Cmnm5s2u | 5-carboxymethylamino-methyl-2- |
| | thioridine |
| Cmnm5u | 5-carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2- |
| | methylthiopurine-6-yl)carbamoyl)threonine |
| Mt6a | N-((9-beta-D-ribofuranosylpurine-6-yl)N- |
| | methyl-carbamoyl)threonine |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6- |
| | yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, |
| | (acp3)u |

A nucleobase may be comprised of a nucleoside or nucleotide, using any chemical or natural synthesis method described herein or known to one of ordinary skill in the art.

2. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i e., C, T or U) typically covalently attaches a 1 position of a pyrimidine to a 1′-position of a 5-carbon sugar.

3. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

4. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moiety, which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moiety replacing phosphodiester backbone moiety used for improved nuclease resistance, cellular uptake and regulating RNA expression; U.S. Pat. No. 5,858,988 which describes hydrophobic carrier agent attached to the 2′-O position of oligonucleotides to enhanced their membrane permeability and stability; U.S. Pat. No. 5,214,136, which describes oligonucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid. Other analogs that may be used with compositions of the invention include U.S. Pat. No. 5,216,141 (discussing oligonucleotide analogs containing sulfur linkages), U.S. Pat. No. 5,432,272 (concerning oligonucleotides having nucleotides with heterocyclic bases), and U.S. Pat. Nos. 6,001,983, 6,037,120, 6,140,496 (involving oligonucleotides with non-standard bases), all of which are incorporated by reference.

5. Polyether and Peptide Nucleic Acids and Locked Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5,891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moiety that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5,891,625. Other modifications and uses of nucleic acid analogs are known in the art, and are encompassed by the bridging and capture nucleic acids of the invention. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. Several alkylamino moieties used to enhance cellular uptake of a PNA are described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

Another non-limiting example is a locked nucleic acid or “LNA.” An LNA monomer is a bicyclic compound that is structurally similar to RNA nucleosides. LNAs have a furanose conformation that is restricted by a methylene linker that connects the 2′-O position to the 4′-C position, as described in Koshkin et al, 1998a and 1998b and Wahlestedt et al., 2000.

6. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

7. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

8. Nucleic Acid Segments

In certain embodiments, the nucleic acid comprises a nucleic acid segment. As used herein, the term “nucleic acid segment,” are smaller fragments of a nucleic acid, such as for non-limiting example, those that correspond to targeted, targeting, bridging, and capture regions. Thus, a “nucleic acid segment” may comprise any part of a gene sequence, of from about 2 nucleotides to the full length of a targeted nucleic acid, capture nucleic acid, or bridging nucleic acid.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

9. Nucleic Acid Complements

The present invention also encompasses a nucleic acid that is complementary to a other nucleic acids of the invention and targeted nucleic acids. More specifically, a targeting region in a bridging nucleic acid is complementary to the targeted region of the targeted nucleic acid and a bridging region of the bridging nucleic acid is complementary to a capture region of a capture nucleic acid. In particular embodiments the invention encompasses a nucleic acid or a nucleic acid segment identical or complementary to all or part of the sequences set forth in SEQ ID NOS: 1-73. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. Unless otherwise specified, a nucleic acid region is “complementary” to another nucleic acid region if there is at least 70, 80%, 90% or 100% Watson-Crick base-pairing (A:T or A:U, C:G) between or between at least 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 or more contiguous nucleic acid bases of the regions. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%, and any range derivable therein, of the nucleobase sequence is capable of base-pairing with a single or double stranded nucleic acid molecule during hybridization, as described in the Examples. In certain embodiments, the term “complementary” refers to a nucleic acid that may hybridize to another nucleic acid strand or duplex under conditions described in the Examples, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

10. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. Alternatively, stringent conditions may be determined largely by temperature in the presence of a TMAC solution with a defined molarity such as 3M TMAC. For example, in 3 M TMAC, stringent conditions include the following: for complementary nucleic acids with a length of 15 bp, a temperature of 45° C. to 55° C.; for complementary nucleotides with a length of 27 bases, a temperature of 65° C. to 75° C.; and, for complementary nucleotides with a length of >200 nucleotides, a temperature of 90° C. to 95° C. The publication of Wood et al., 1985, which is specifically incorporated by reference, provides examples of these parameters. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

11. Oligonucleotide Synthesis

Oligonucleotide synthesis is performed according to standard methods. See, for example, Itakura and Riggs (1980). Additionally, U.S. Pat. No. 4,704,362; U.S. Pat. No. 5,221,619, U.S. Pat. No. 5,583,013 each describe various methods of preparing synthetic structural genes.

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers. (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligodeoxynucleotides (Gillam et al., 1978; Gillam et al., 1979). Under controlled conditions, polynucleotide phosphorylase adds predominantly a single nucleotide to a short oligodeoxynucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-phase methods. Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic DNA synthesizers.

Phosphoramidite chemistry (Beaucage, and Lyer, 1992) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

12. Expression Vectors

Other ways of creating nucleic acids of the invention include the use of a recombinant vector created through the application of recombinant nucleic acid technology known to those of skill in the art or as described herein. A recombinant vector may comprise a bridging or capture nucleic acid, particularly one that is a polynucleotide, as opposed to an oligonucleotide. An expression vector can be used create nucleic acids that are lengthy, for example, containing multiple targeting regions or relatively lengthy targeting regions, such as those greater than 100 residues in length.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrook et al, 2001 and Ausubel et al., 1994, both incorporated herein by reference).

The term “expression vector” refers to any type of genetic construct comprising a nucleic acid coding for a RNA capable of being transcribed. Expression vectors can contain a variety of “control sequences,” which refer to nucleic acid sequences necessary for the transcription and possibly translation of an operable linked coding sequence in a particular host cell. In addition to control sequences that govern transcription (promoters and enhancers) and translation, vectors and expression vectors may contain nucleic acid sequences that serve other functions as well that are well known to those of skill in the art, such as screenable and selectable markers, ribosome binding site, multiple cloning sites, splicing sites, poly A sequences, origins of replication, and other sequences that allow expression in different hosts.

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The nucleotide and protein, polypeptide and peptide sequences for various genes have been previously disclosed, and may be found at computerized databases known to those of ordinary skill in the art. For example, the nucleotide sequences of rRNAs of various organisms are readily available. One such database is the National Center for Biotechnology Information's Genbank and GenPept databases (http://www.ncbi.nlm.nih.gov/). The coding regions for all or part of these known genes may be amplified and/or expressed using the techniques disclosed herein or by any technique that would be know to those of ordinary skill in the art.

13. Nucleic Acid Arrays

Because the present invention provides efficient methods of enriching in mRNA, which can be used to make cDNA, the present invention extends to the use of cDNAs with arrays. The term “array” as used herein refers to a systematic arrangement of nucleic acid. For example, a cDNA population that is representative of a desired source (e.g., human adult brain) is divided up into the minimum number of pools in which a desired screening procedure can be utilized to detect a cDNA and which can be distributed into a single multi-well plate. Arrays may be of an aqueous suspension of a cDNA population obtainable from a desired mRNA source, comprising: a multi-well plate containing a plurality of individual wells, each individual well containing an aqueous suspension of a different content of a cDNA population. The cDNA population may include cDNA of a predetermined size. Furthermore, the cDNA population in all the wells of the plate may be representative of substantially all mRNAs of a predetermined size from a source. Examples of arrays, their uses, and implementation of them can be found in U.S. Pat. Nos. 6,329,209, 6,329,140, 6,324,479, 6,322,971, 6,316,193, 6,309,823, 5,412,087, 5,445,934, and 5,744,305, which are herein incorporated by reference.

The number of cDNA clones array on a plate may vary. For example, a population of cDNA from a desired source can have about 200,000-6,000,000 cDNAs, about 200,000-2,000,000, 300,000-700,000, about 400,000-600,000, or about 500,000 cDNAs, and combinations thereof. Such a population can be distributed into a small set of multi-well plates, such as a single 96-well plate or a single 384-well plate. For instance, when about 1000-10,000 cDNAs, preferably about 3,500-7,000, more preferably about 5,000, from a population are present in a single well of a 96-well or 384-well plate, PCR can be utilized to clone a single, target gene using a set of primers.

The term a “nucleic acid array” refers to a plurality of target elements, each target element comprising one or more nucleic acid molecules immobilized on one or more solid surfaces to which sample nucleic acids can be hybridized. The nucleic acids of a target element can contain sequence(s) from specific genes or clones, e.g. from the regions identified here. Other target elements will contain, for instance, reference sequences. Target elements of various dimensions can be used in the arrays of the invention. Generally, smaller, target elements are preferred. Typically, a target element will be less than about 1 cm in diameter. Generally element sizes are from 1 μm to about 3 mm, between about 5 μm and about 1 mm. The target elements of the arrays may be arranged on the solid surface at different densities. The target element densities will depend upon a number of factors, such as the nature of the label, the solid support, and the like. One of skill will recognize that each target element may comprise a mixture of nucleic acids of different lengths and sequences. Thus, for example, a target element may contain more than one copy of a cloned piece of DNA, and each copy may be broken into fragments of different lengths. The length and complexity of the nucleic acid fixed onto the target element is not critical to the invention. One of skill can adjust these factors to provide optimum hybridization and signal production for a given hybridization procedure, and to provide the required resolution among different genes or genomic locations. In various embodiments, target element sequences will have a complexity between about 1 kb and about 1 Mb, between about 10 kb to about 500 kb, between about 200 to about 500 kb, and from about 50 kb to about 150 kb.

Microarrays are known in the art and consist of a surface to which probes that correspond in sequence to gene products (e.g., cDNAs, mRNAs, cRNAs, polypeptides, and fragments thereof), can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

A microarray may contains binding sites for products of all or almost all genes in the target organism's genome, but such comprehensiveness is not necessarily required. Usually the microarray will have binding sites corresponding to at least about 50% of the genes in the genome, often at least about 75%, more often at least about 85%, even more often more than about 90%, and most often at least about 99%. Preferably, the microarray has binding sites for genes relevant to the action of a drug of interest or in a biological pathway of interest. A “gene” is identified as an open reading frame (ORF) of preferably at least 50, 75, or 99 amino acids from which a messenger RNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence.

The nucleic acid or analogue are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995a. See also DeRisi et al., 1996; Shalon et al., 1996; Schena et al., 1995b. Each of these articles is incorporated by reference in its entirety.

Other methods for making microarrays, e.g., by masking (Maskos et al., 1992), may also be used. In principal, any type of array, for example, dot blots on a nylon hybridization membrane (see Sambrook et al., 1989, which is incorporated in its entirety for all purposes), could be used, although, as will be recognized by those of skill in the art, very small arrays will be preferred because hybridization volumes will be smaller.

Labeled cDNA is prepared from mRNA by oligo dT-primed or random-primed reverse transcription, both of which are well known in the art (see e.g., Klug et al., 1987). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, which is incorporated by reference in its entirety for all purposes). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

Fluorescently-labeled probes can be used, including suitable fluorophores such as fluorescein, lissamine, phycoerythrin, rhodamine (Perkin Elmer Cetus), Cy2, Cy3, Cy3.5, Cy5, Cy5.5, Cy7, Fluor X (Amersham) and others (see, e.g., Kricka, 1992). It will be appreciated that pairs of fluorophores are chosen that have distinct emission spectra so that they can be easily distinguished. In another embodiment, a label other than a fluorescent label is used. For example, a radioactive label, or a pair of radioactive labels with distinct emission spectra, can be used (see Zhao et al., 1995; Pietu et al., 1996). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript™, Invitrogen Inc.) at 42° C. for 60 min.

III. Methods for Isolating and Depleting Targeted Nucleic Acids

Methods of the invention involve preparing a sample comprising a targeted nucleic acid, preparing a bridging nucleic acid, preparing a capture nucleic acid, incubating the sample with the bridging nucleic acid, incubating the sample with a capture nucleic acid, incubating the bridging nucleic acid with the capture nucleic acid, incubating compounds under conditions allowing for hybridization among complementary regions, washing the sample and/or the capture and/or bridging nucleic acids, and isolating the capture nucleic acids and any accompanying compounds (compounds that bind or hybridize directly or indirectly to the capture nucleic acids). Steps of the invention are not required to be in a particular order and thus, the invention covers methods in which the order of the steps varies.

Hybridization conditions are discussed earlier. Wash conditions may involve temperatures between 20° C. and 75° C., between 25° C. and 70° C., between 30° C. and 65° C., between 35° C. and 60° C., between 40° C. and 55° C., between 45° C. and 50° C., or at temperatures within the ranges specified.

Buffer conditions for hybridization of nucleic acid compositions are well known to those of skill in the art. It is specifically contemplated that isostabilizing agents may be employed in hybridization and wash buffers in methods of the invention. U.S. Ser. No. 09/854,412 describes the use of tetramethylammonium chloride (TMAC) and tetraethylammonium chloride (TEAC) in such buffers; this application is specifically incorporated by reference herein. The concentration of an isostabilizing agent in a hybridization (binding) buffer may be between about 1.0 M and about 5.0 M, is about 4.0 M, or is about 2.0 M. Also specifically contemplated is a wash solution with an isostabilizing agent concentration of between about 0.1 M and 3.0 M, including 0.1 M increments within the range. Wash buffers may or may not contain Tris. However, in some embodiments of the invention, the wash solution consists of water and no other salts or buffers. In some embodiments of the invention, the hybridizing or wash buffer may include guanidinium isothiocyanate, though in some embodiments this chemical is specifically contemplated to be absent. The concentration of guanidinium may be between about 0.4 M and about 3.0 M

A solution or buffer to elute targeted nucleic acids from the hybridizing nucleic acids (indirect or direct) may be implemented in some kits and methods of the invention. The elution buffer or solution can be an aqueous solution lacking salt, such as TE or water. Elution may occur at room temperature or it may occur at temperatures between 15° C. and 100° C., between 20° C. and 95° C., between 25° C. and 90° C., between 30° C. and 85° C., between 35° C. and 80° C., between 40° C. and 75° C., between 45° C. and 70° C., between 50° C. and 65° C., between 55° C. and 60° C., or at temperatures within the ranges specified.

A. Quantitation of RNA

1. Assessing RNA Yield by UV Absorbance

The concentration and purity of RNA can be determined by diluting an aliquot of the preparation (usually a 1:50 to 1:100 dilution) in TE (10 mM Tris-HCl pH 8, 1 mM EDTA) or water, and reading the absorbance in a spectrophotometer at 260 nm and 280 nm.

An A₂₆₀ of 1 is equivalent to 40 μg RNA/ml. The concentration (μg/ml) of RNA is therefore calculated by multiplying the A₂₆₀ X dilution factor X 40 μg/ml. The following is a typical example:

The typical yield from 10 μg total RNA is 3-5 μg. If the sample is re-suspended in 25 μl, this means that the concentration will vary between 120 ng/μl and 200 ng/μl. One μl of the prep is diluted 1:50 into 49 μl of TE. The A₂₆₀=0.1. RNA concentration=0.1×50×40 μg/ml=200 μg/ml or 0.2 μg/μl. Since there are 24 μl of the prep remaining after using 1 μl to measure the concentration, the total amount of remaining RNA is 24 μl×0.2 μg/μl=4.8 μg.

2. Assessing RNA Yield with RiboGreen®

Molecular Probes' RiboGreen® fluorescence-based assay for RNA quantitation can be employed to measure RNA concentration.

B. Denaturing Agarose Gel Electrophoresis

Many mRNAs form extensive secondary structure. Ribosomal RNA depletion may be evaluated by agarose gel electrophoresis. Because of this, it is best to use a denaturing gel system to analyze RNA samples. A positive control should be included on the gel so that any unusual results can be attributed to a problem with the gel or a problem with the RNA under analysis. RNA molecular weight markers, an RNA sample known to be intact, or both, can be used for this purpose. It is also a good idea to include a sample of the starting RNA that was used in the enrichment procedure.

Ambion's NorthernMax™ reagents for Northern Blotting include everything needed for denaturing agarose gel electrophoresis. These products are optimized for ease of use, safety, and low background, and they include detailed instructions for use. An alternative to using the NorthernMax reagents is to use a procedure described in “Current Protocols in Molecular Biology”, Section 4.9 (Ausubel et al., eds.), hereby incorporated by reference. It is more difficult and time-consuming than the Northern-Max method, but it gives similar results.

C. Agilent 2100 Bioanalyzer

1. Evaluating rRNA Removal with the RNA 6000 LabChip

An effective method for evaluating rRNA removal utilizes RNA analysis with the Caliper RNA 6000 LabChip Kit and the Agilent 2100 Bioanalayzer. Follow the instructions provided with the RNA 6000 LabChip Kit for RNA analysis. This system performs best with RNA solutions at concentrations between 50 and 250 ng/μl. Loading 1 μl of a typical enriched RNA sample is usually adequate for good performance.

2. Expected Results

In enriched mRNA samples from prokaryotes, the 16S and 23S rRNA peaks will be absent or present in only very small amounts. The peak calling feature of the software may fail to identify the peaks containing small quantities of leftover 16S and 23S rRNAs. A peak corresponding to 5S and tRNAs may be present depending on how the total RNA was initially purified. If RNA was purified by a glass fiber filter method prior to enrichment, this peak will be smaller. The size and shape of the 5S rRNA-tRNA peak is unchanged by some embodiments.

IV. Kits

Any of the compositions described herein may be comprised in a kit. In a non-limiting example, a bridging nucleic acid and a capture nucleic acid may be comprised in a kit. The kits will thus comprise, in suitable container means, a bridging nucleic acid and a capture nucleic of the present invention. It may also include one or more buffers, such as hybridization buffer or a wash buffer, compounds for preparing the sample, and components for isolating the capture nucleic acid via the nonreacting structure. Other kits of the invention may include components for making a nucleic acid array, and thus, may include, for example, a solid support.

The kits may comprise suitably aliquoted nucleic acid compositions of the present invention, whether labeled or unlabeled, as may be used to isolate, deplete, or separate a targeted nucleic acid. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (bridging and capture nucleic acids may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Such kits may also include components that facilitate isolation of the targeting molecule, such as filters, beads, or a magnetic stand. Such kits generally will comprise, in suitable means, distinct containers for each individual reagent or solution as well as for the targeting agent.

A kit will also include instructions for employing the kit components as well the use of any other reagent not included in the kit. Instructions may include variations that can be implemented.

Kits of the invention may also include one or more of the following, in addition to a capture nucleic acid and a bridging nucleic acid:

1) Control RNA (E. coli or other appropriate RNA);

2) Nuclease-free water;

3) RNase-free containers, such as 1.5 ml tubes;

4) RNase-free elution tubes;

5) glycogen;

6) ethanol;

7) sodium acetate;

8) ammonium acetate;

9) magnetic stand or other magnetic field;

10) agarose;

11) nucleic acid size marker;

12) RNase-free tube tips;

13) and RNase or DNase inhibitors.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Furthermore, these examples are provided as one of many ways of implementing the claimed method and using the compositions of the invention. It is contemplated that the invention is not limited to the specific conditions set forth below, but that the conditions below provide examples of how to implement the invention.

### Example 1

### Materials

The following materials were used in the methods described herein for the selective removal of 16S and 23S rRNA and/or 18S and 28S rRNA, and hence mRNA enrichment, from total RNA. All steps are performed at room temperature unless otherwise indicated.

1. Bridging Nucleic Acids

In the following examples, the bridging regions are the poly-A stretches in the respective oligonucleotides.

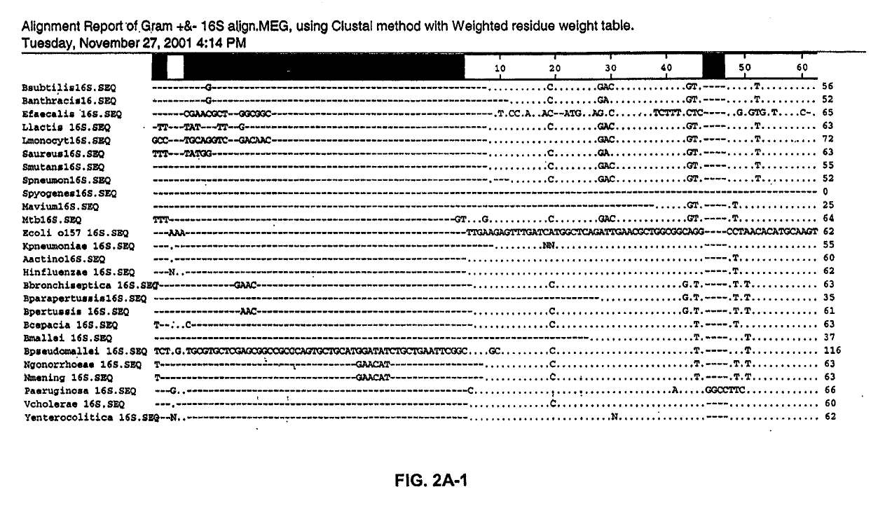

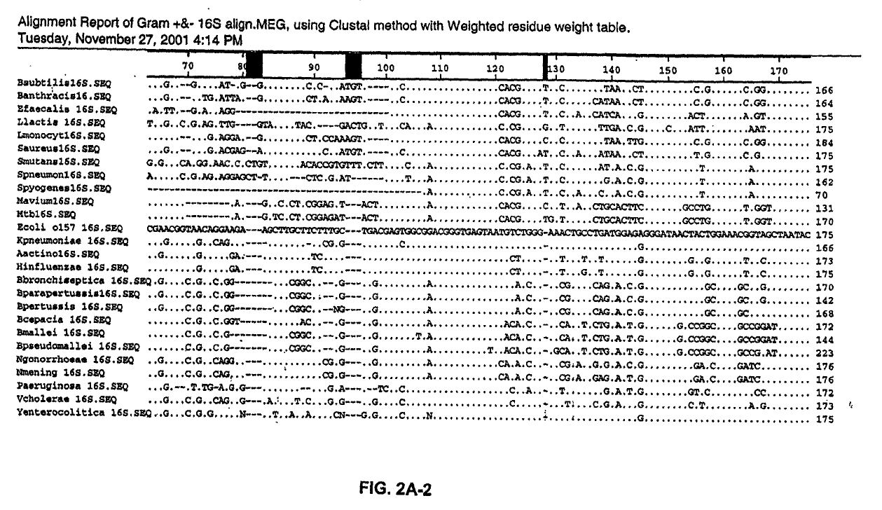

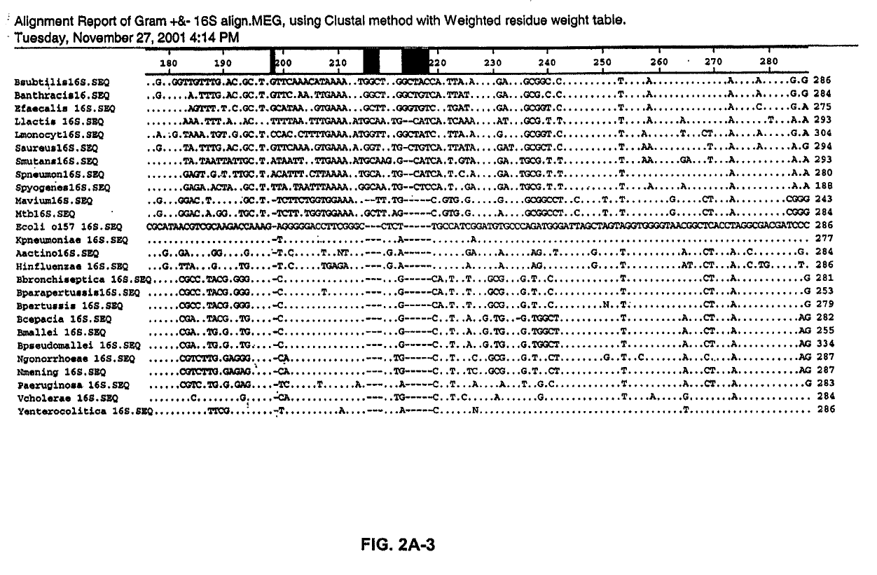

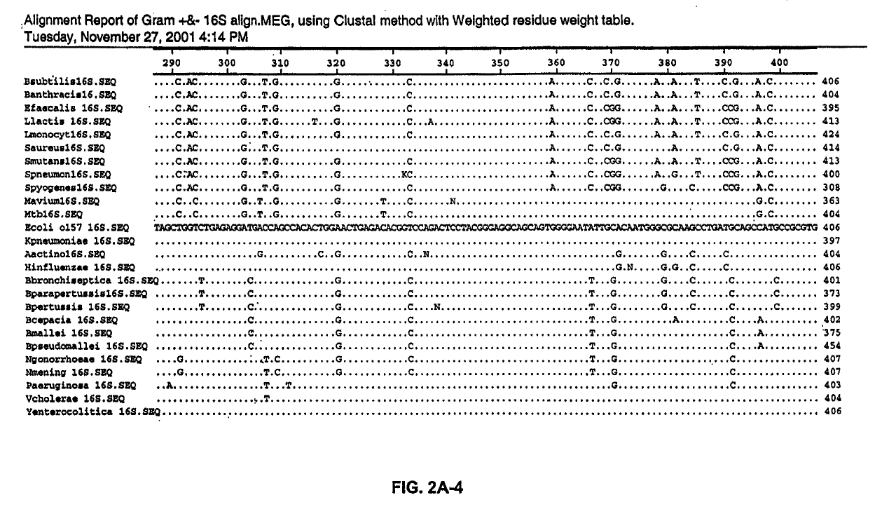

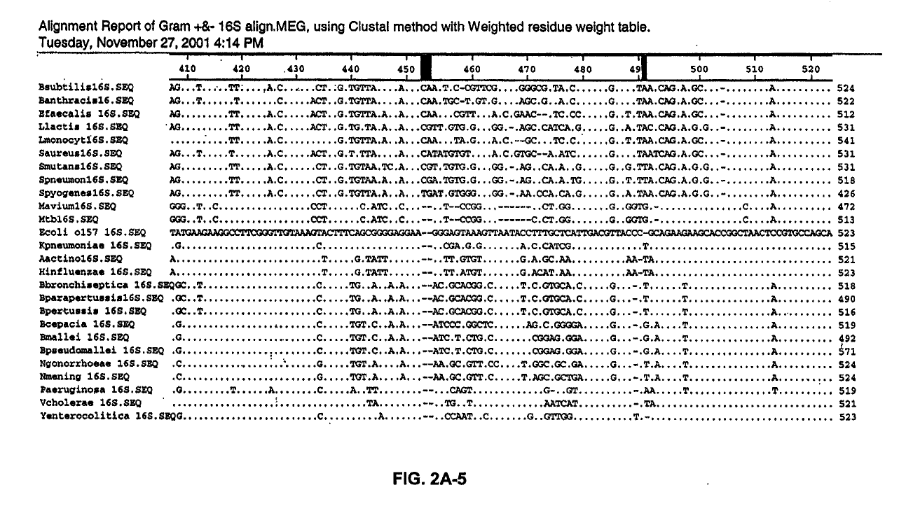

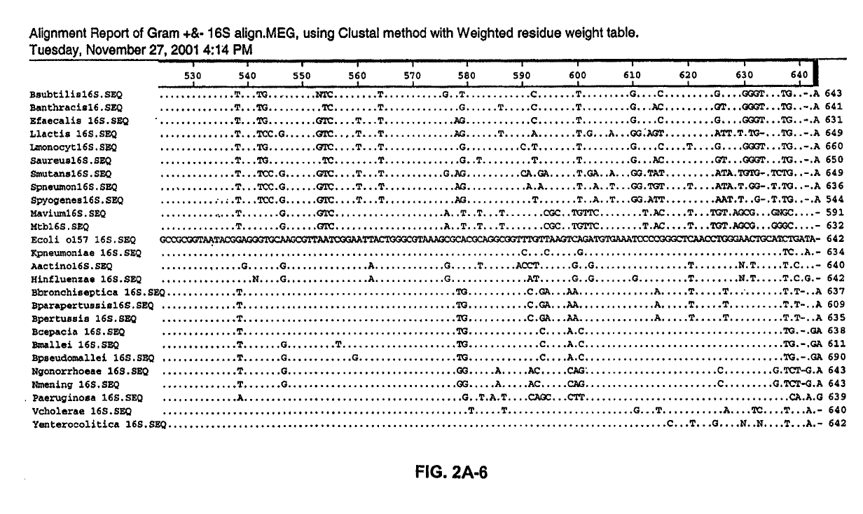

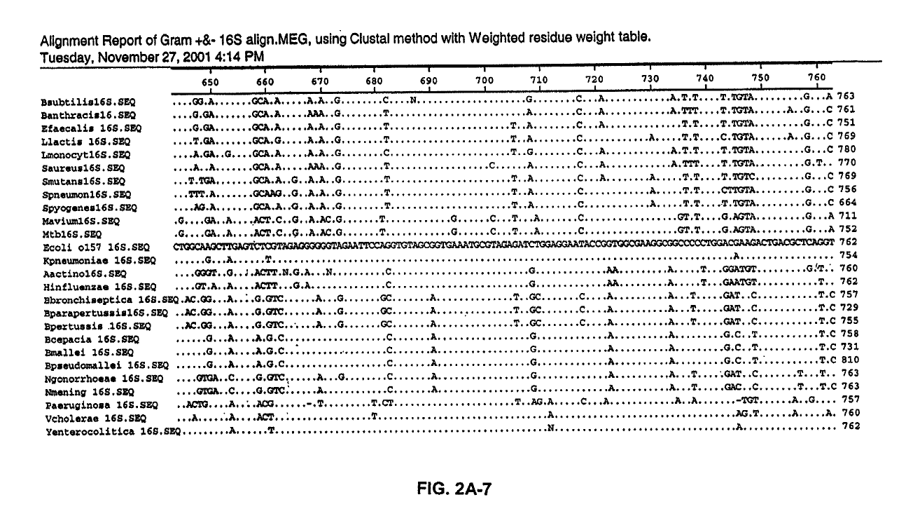

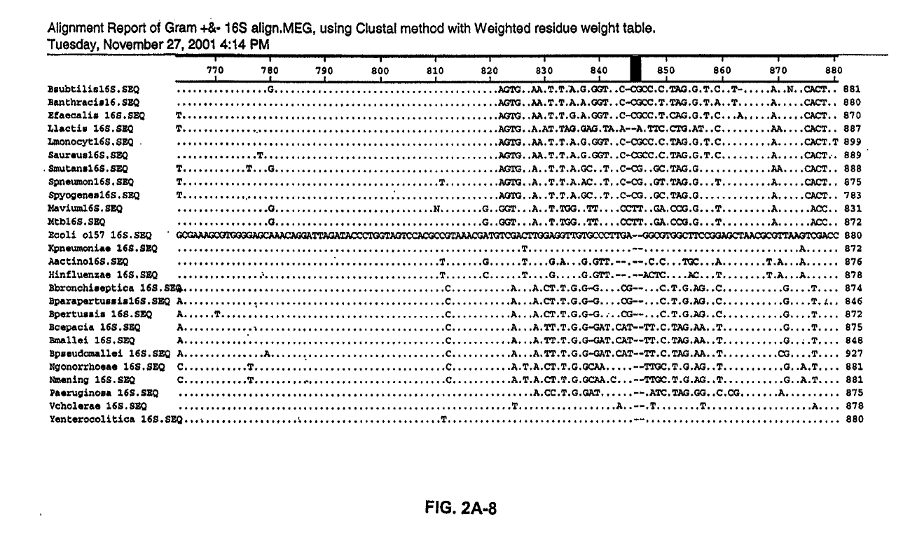

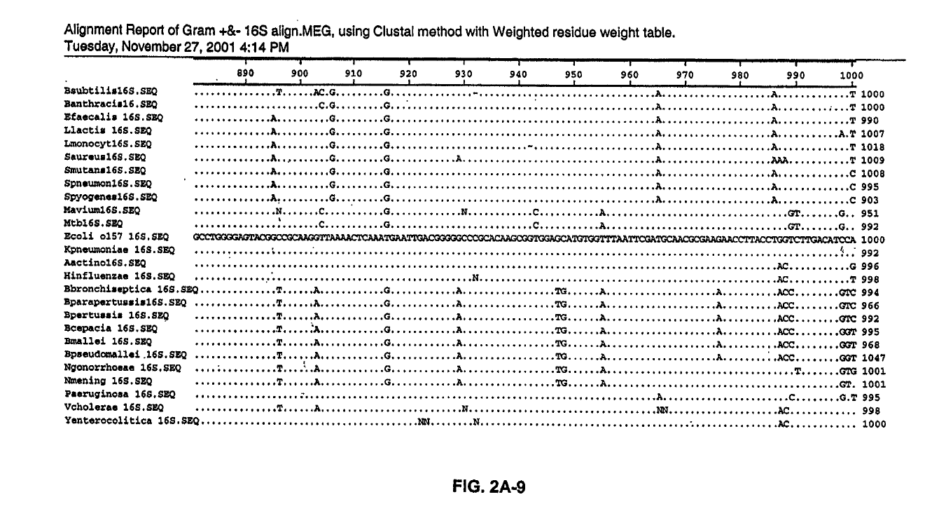

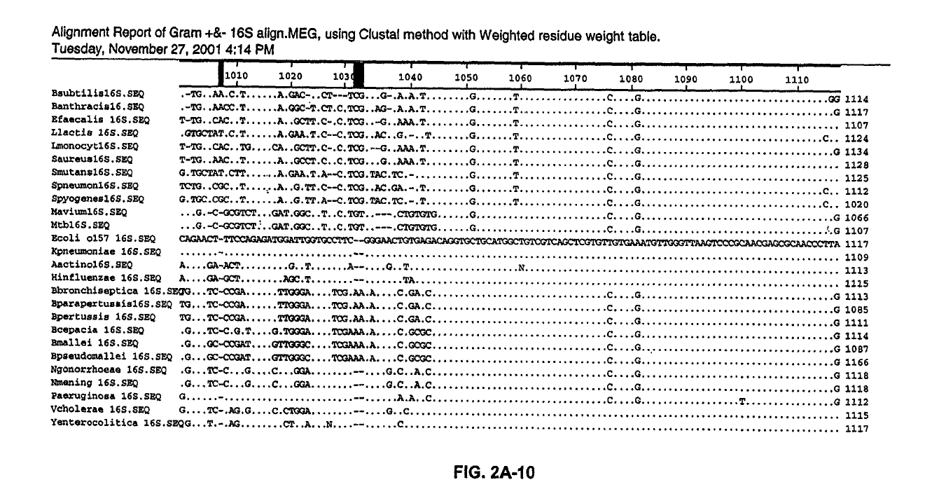

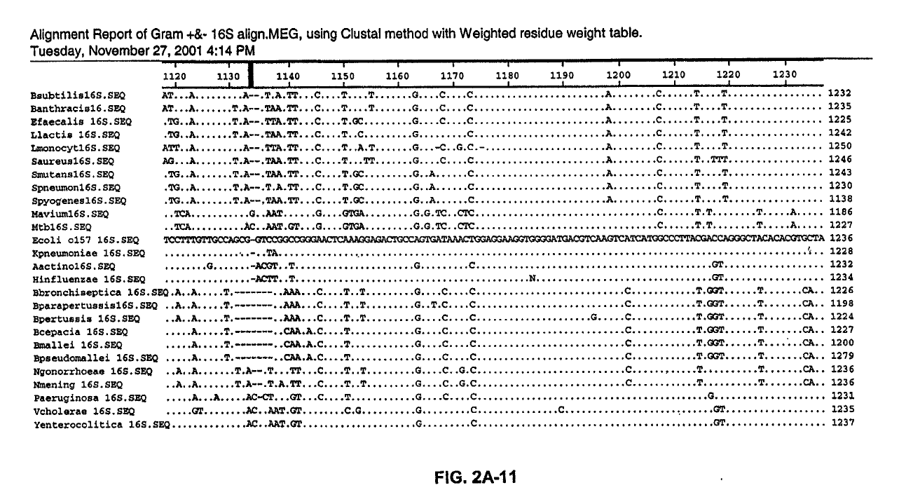

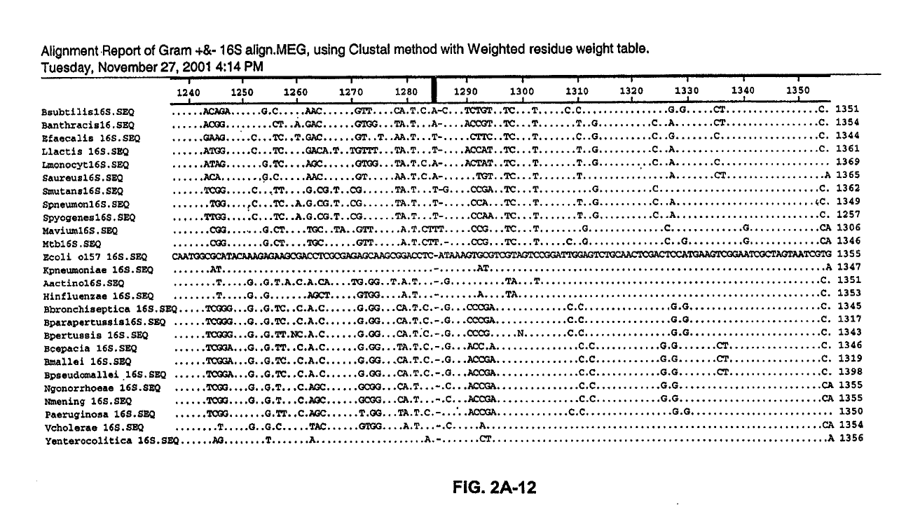

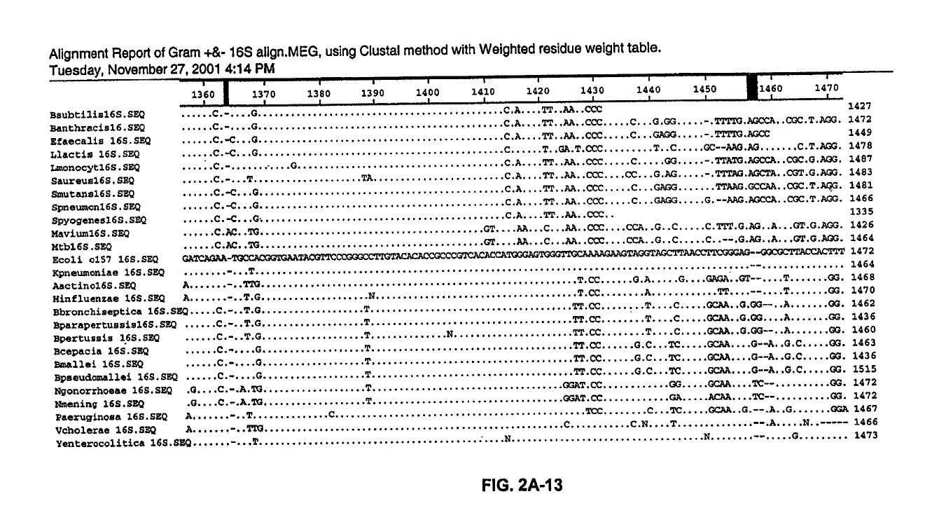

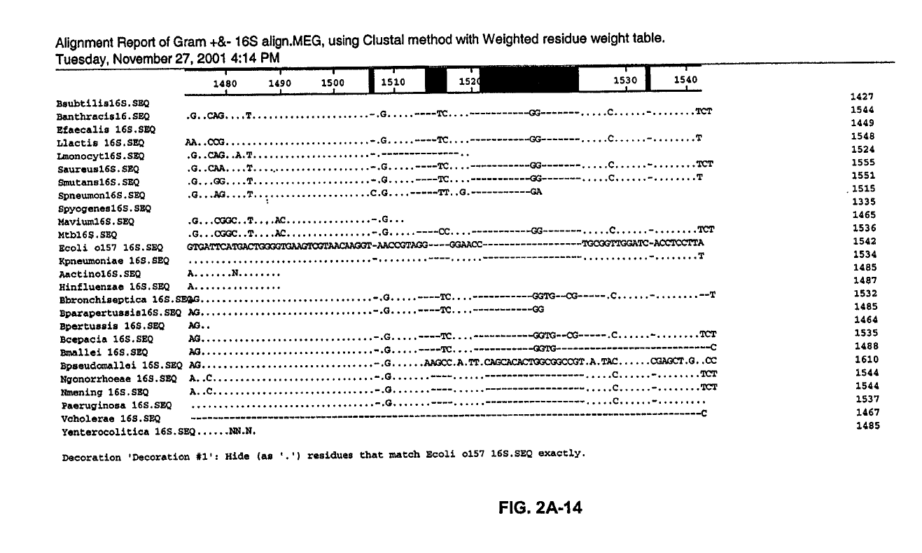

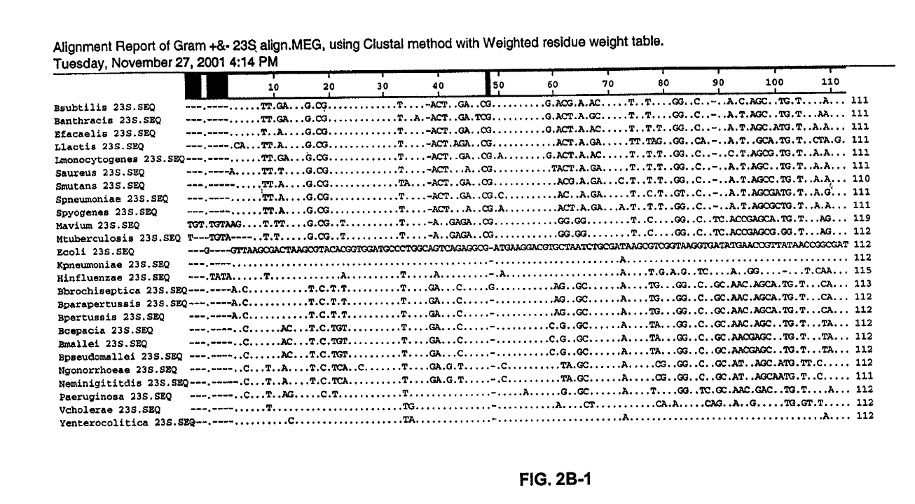

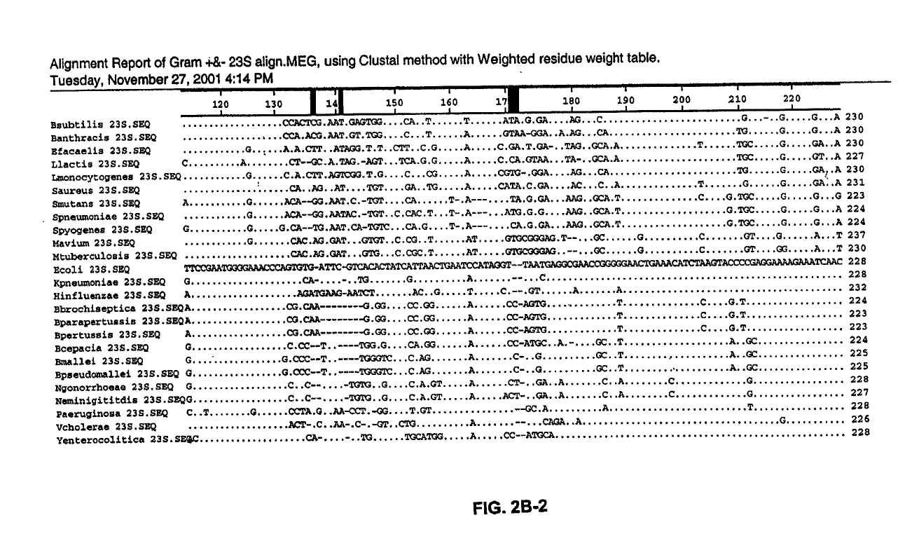

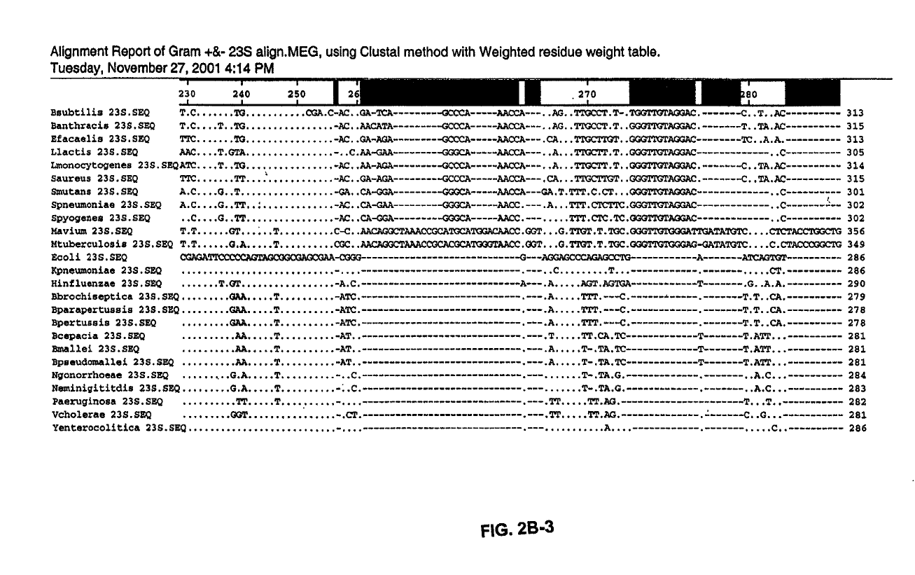

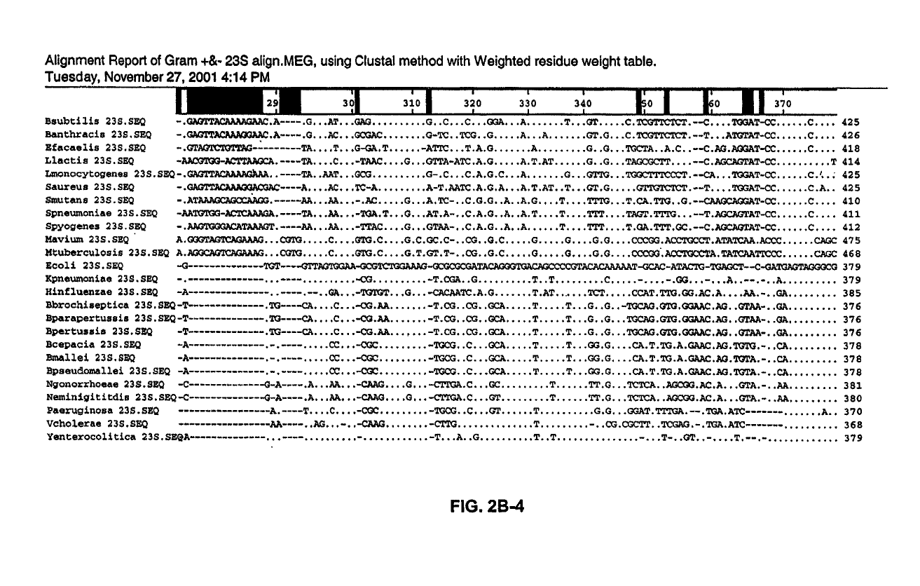

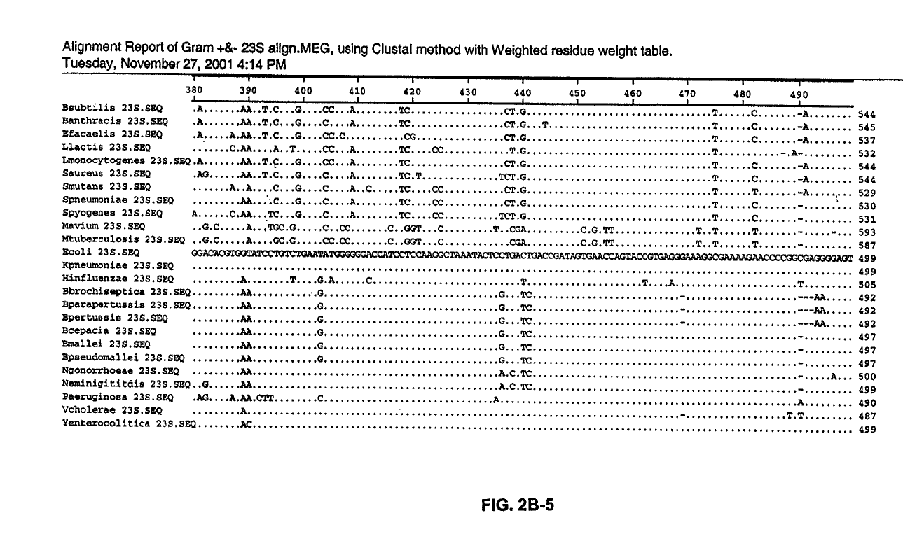

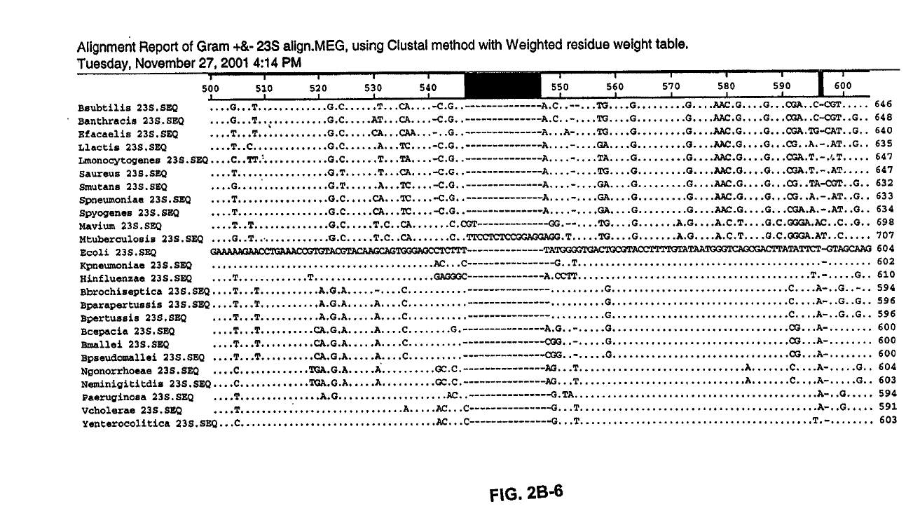

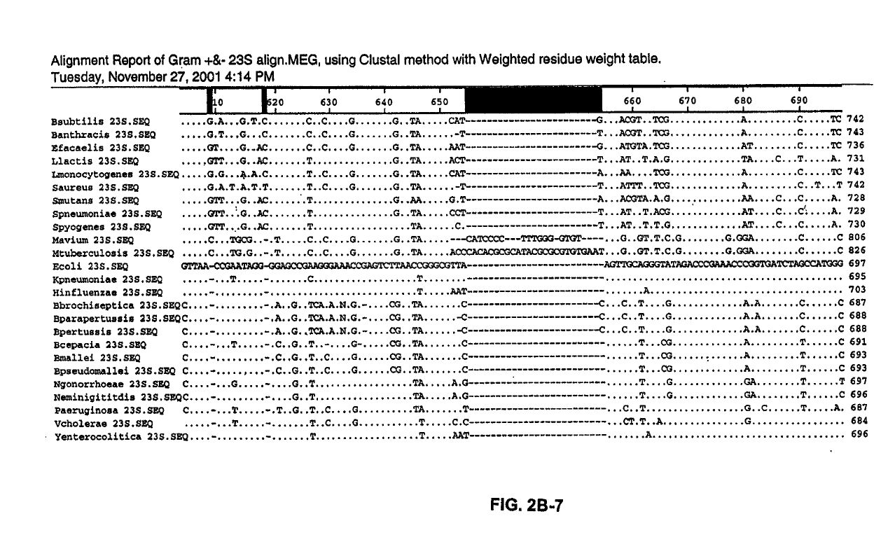

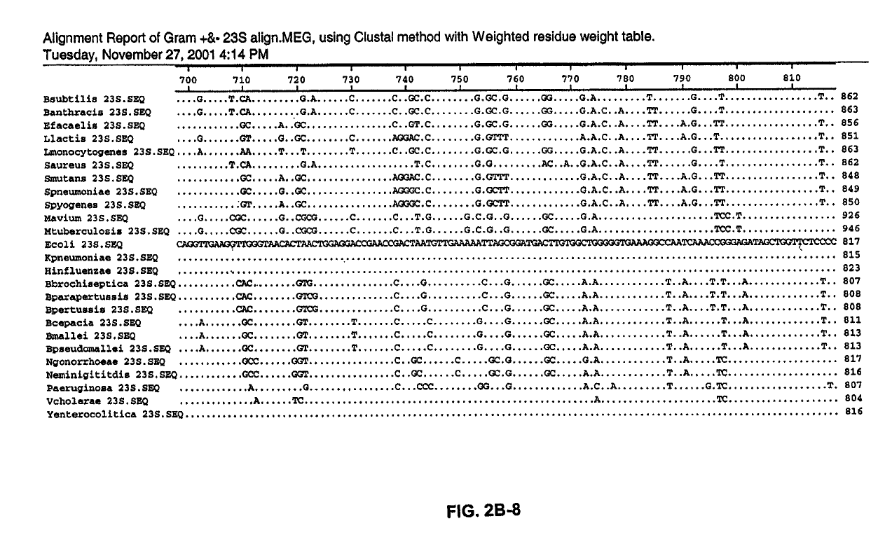

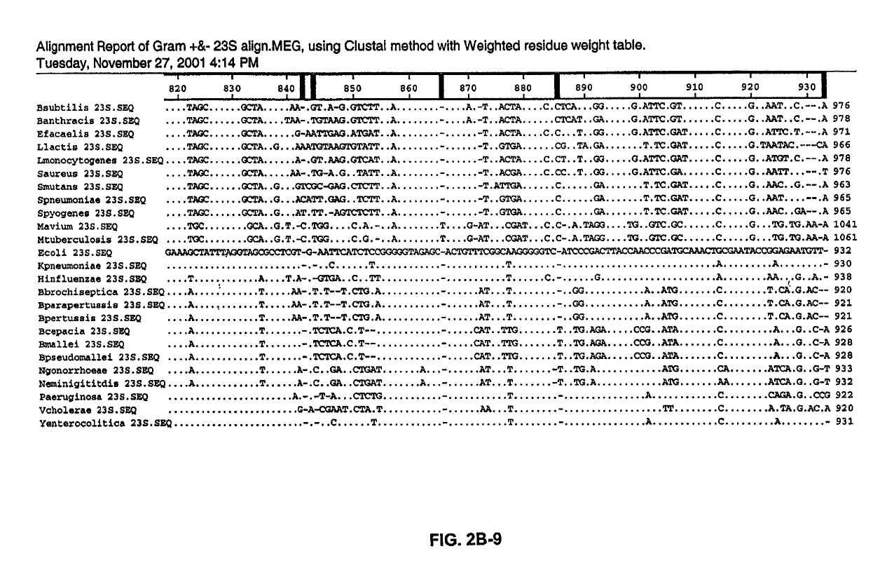

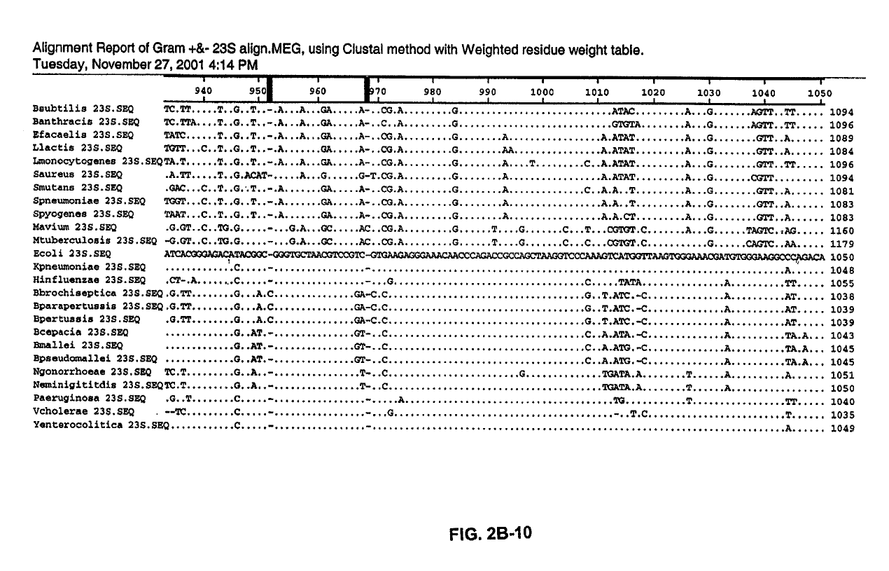

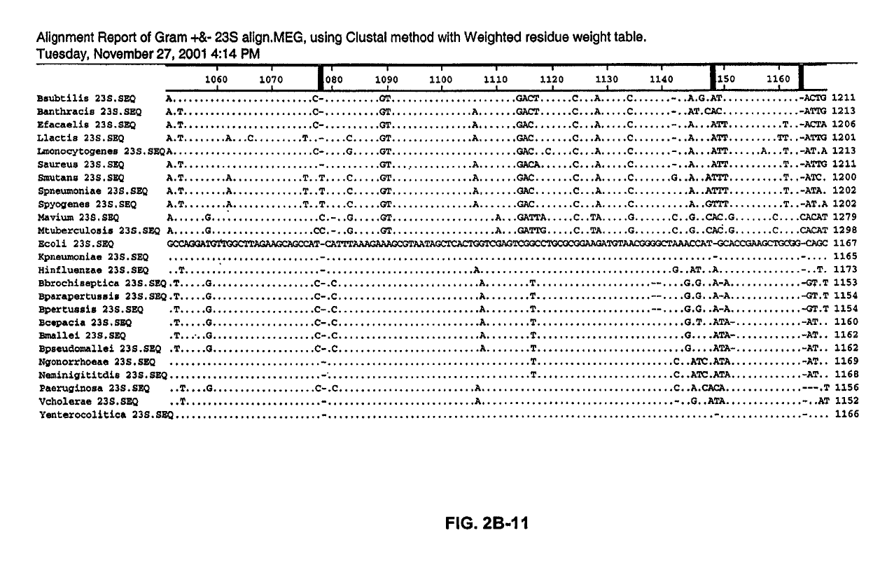

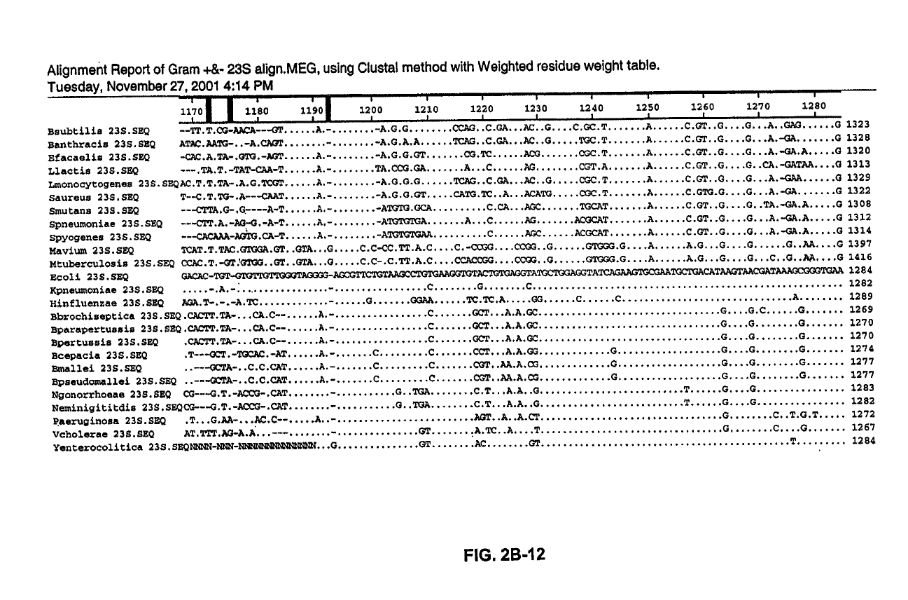

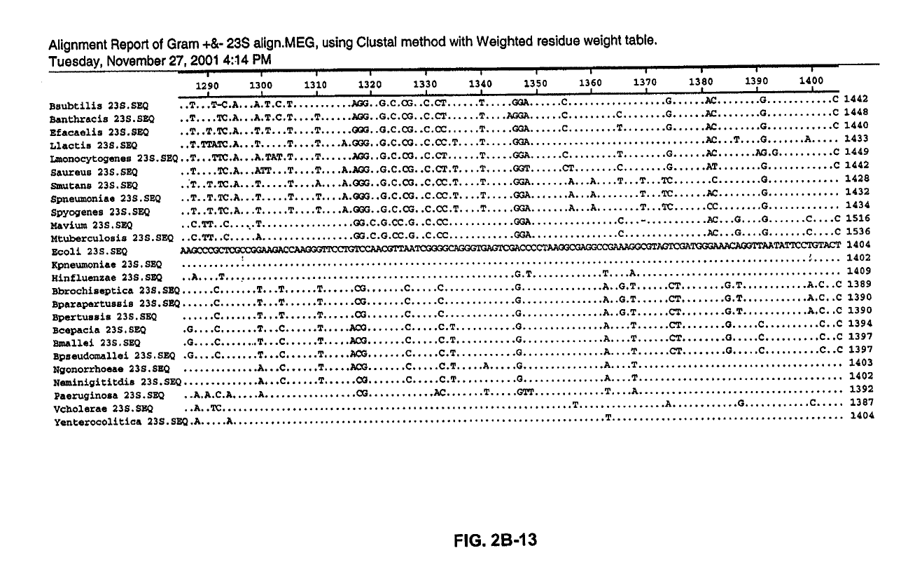

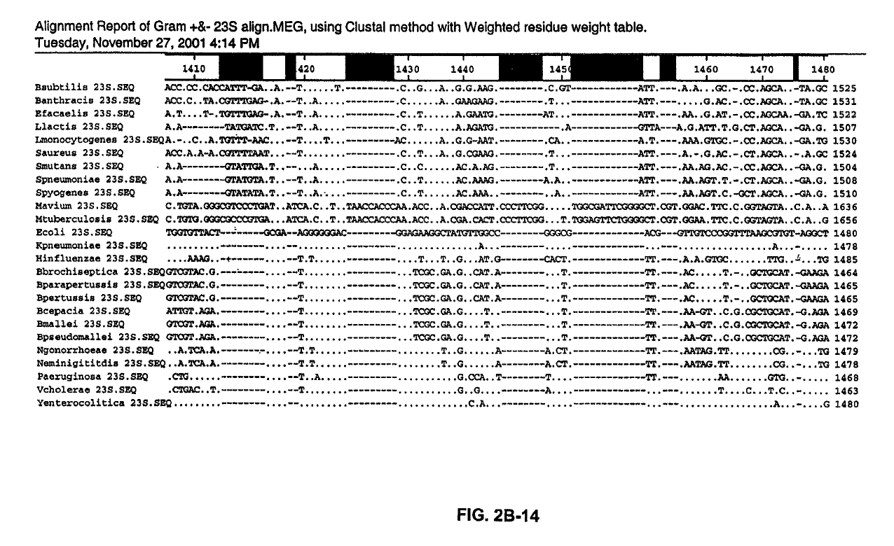

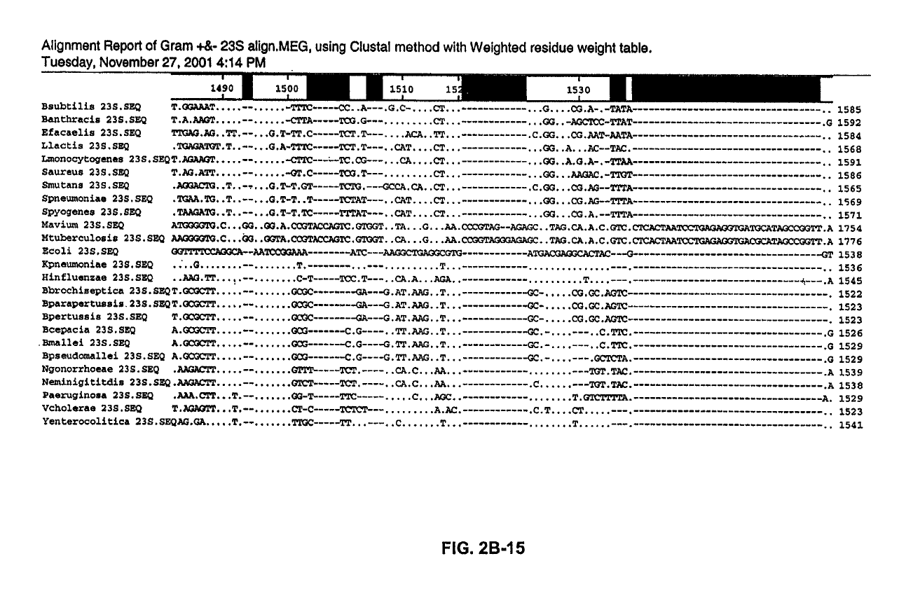

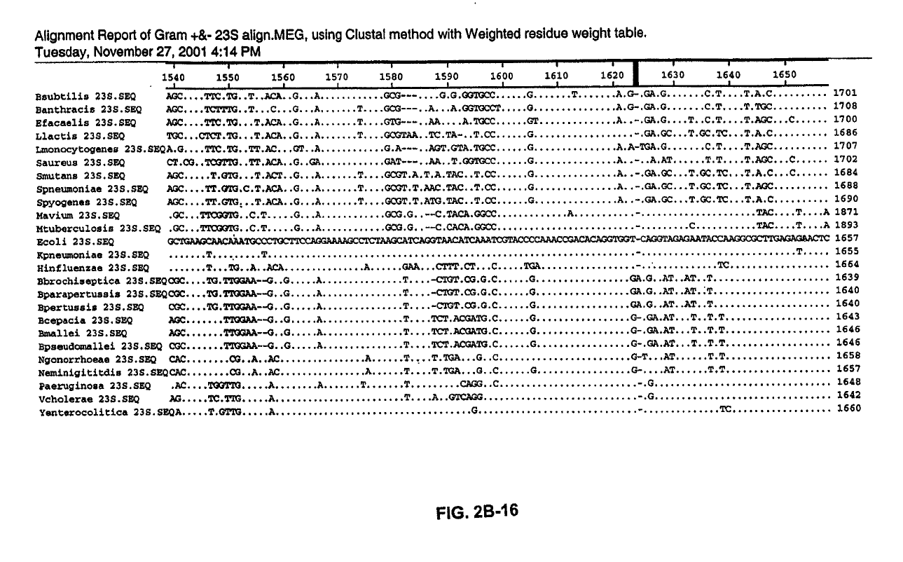

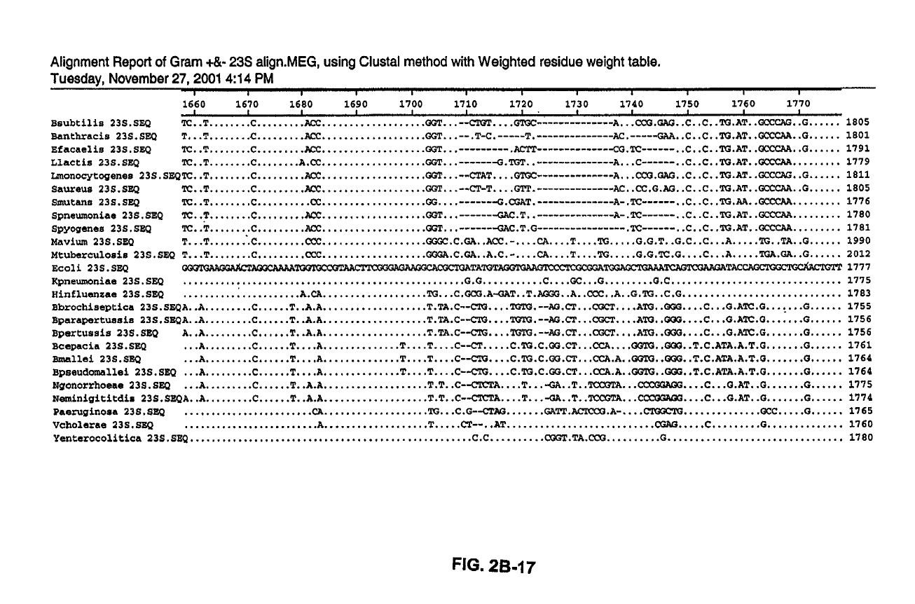

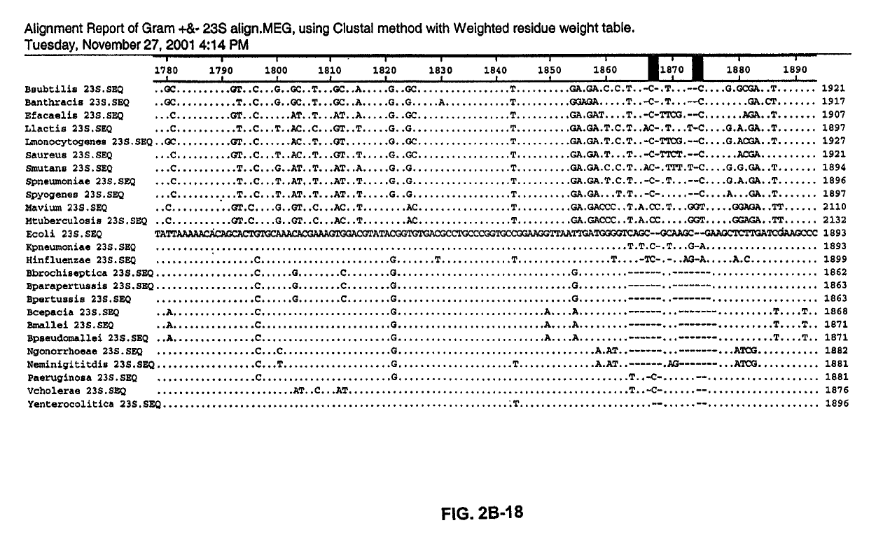

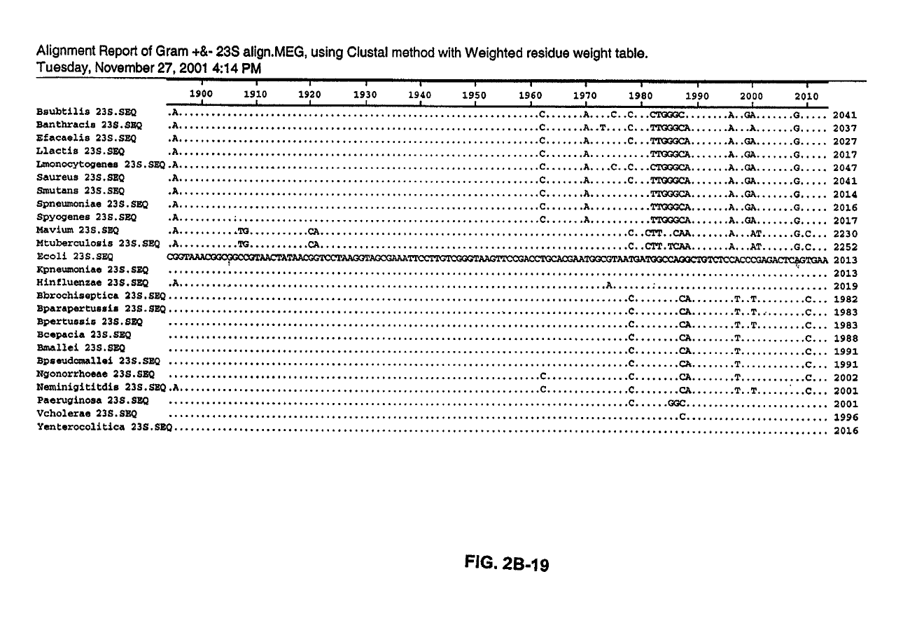

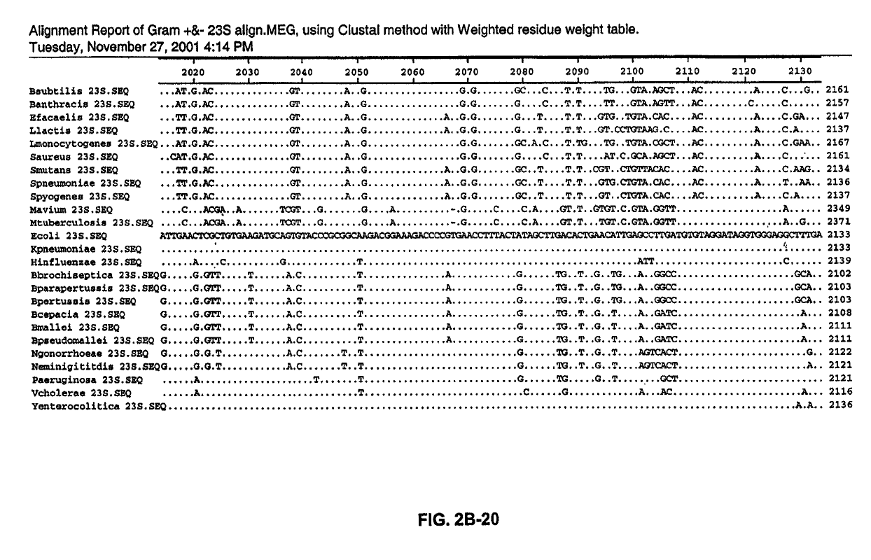

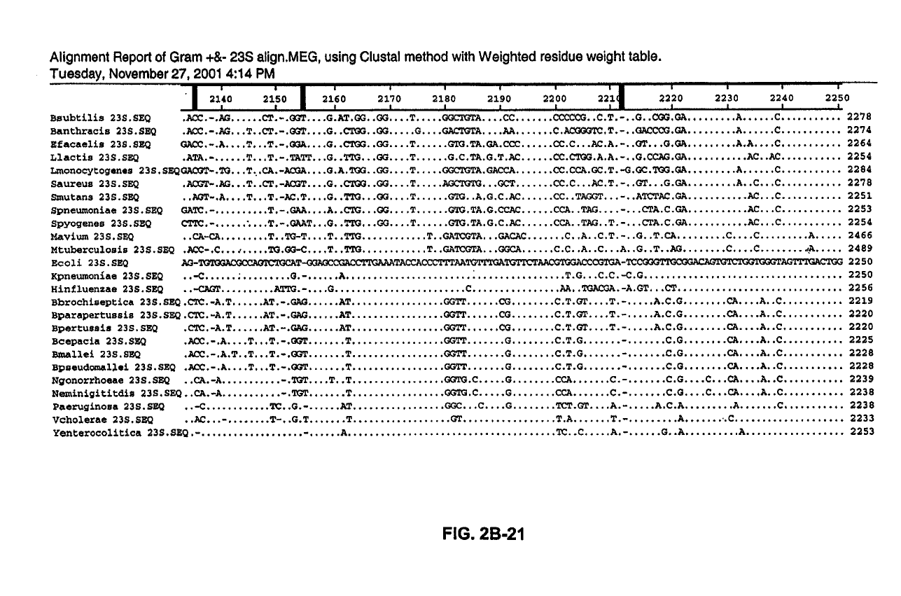

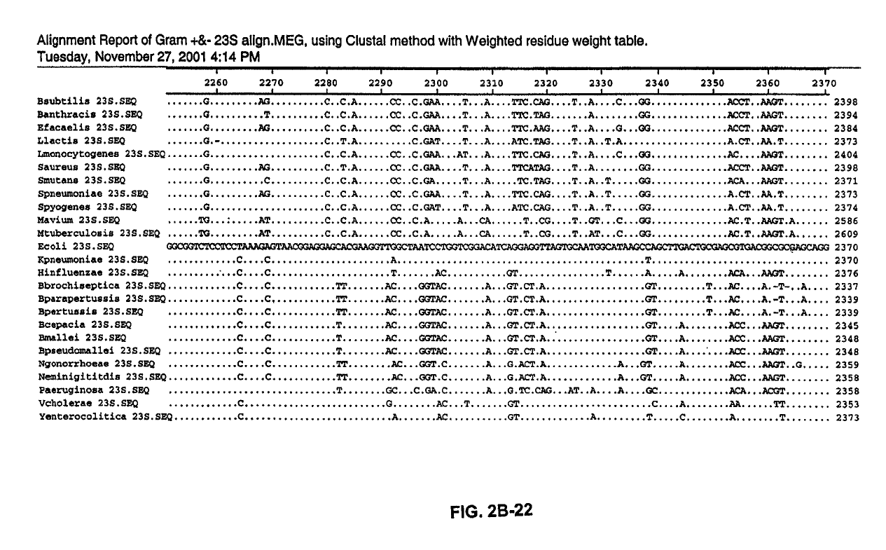

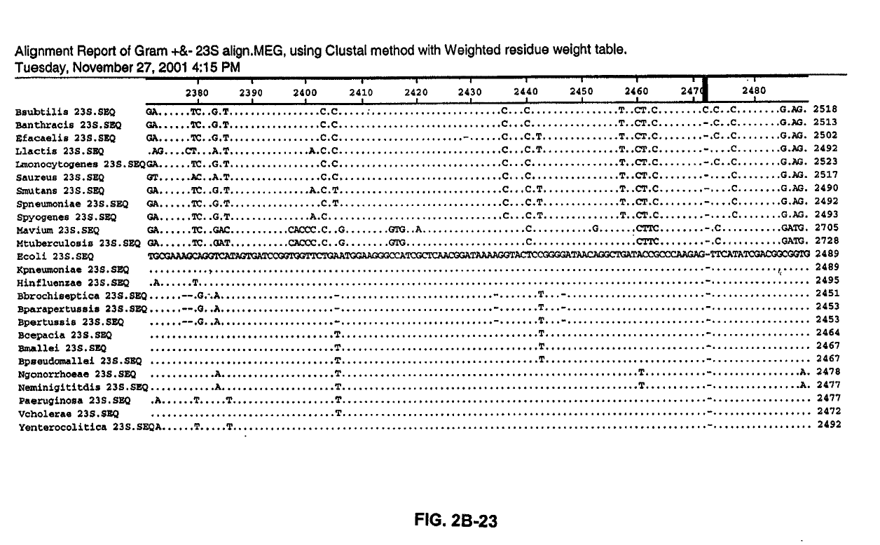

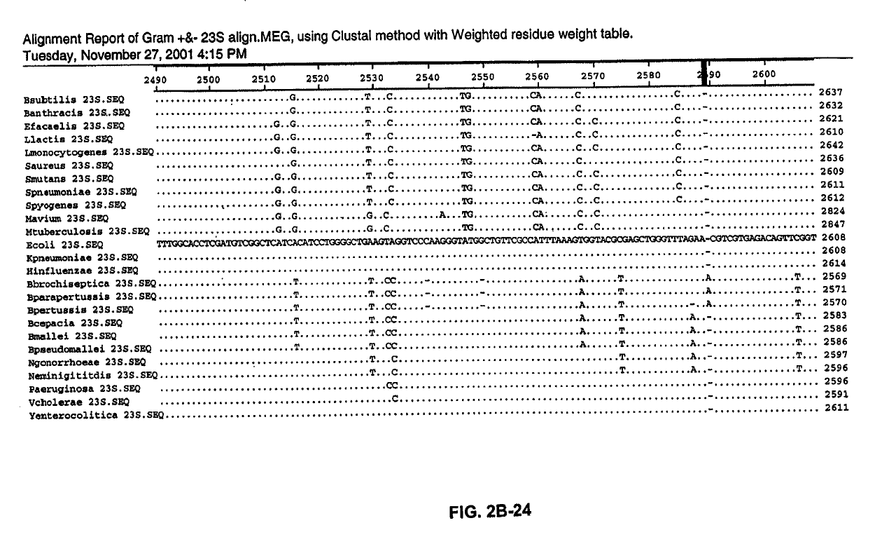

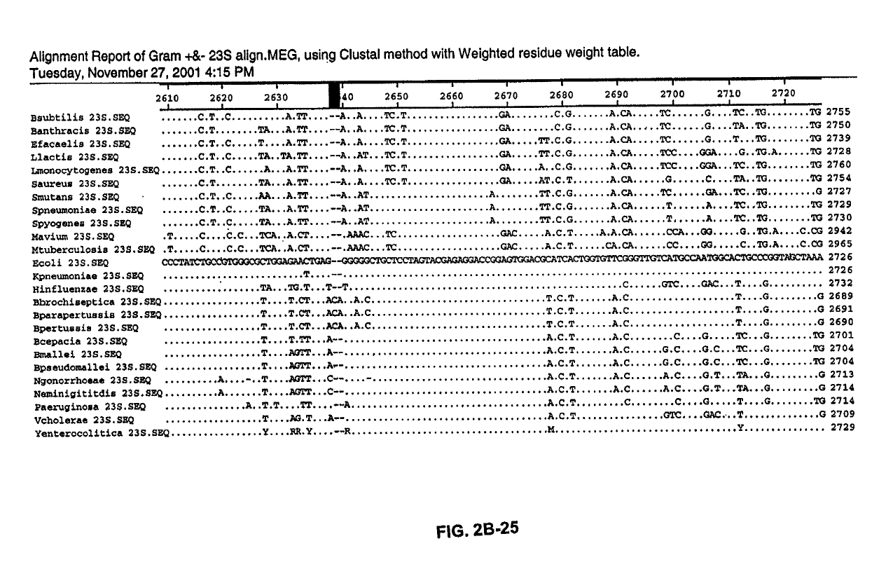

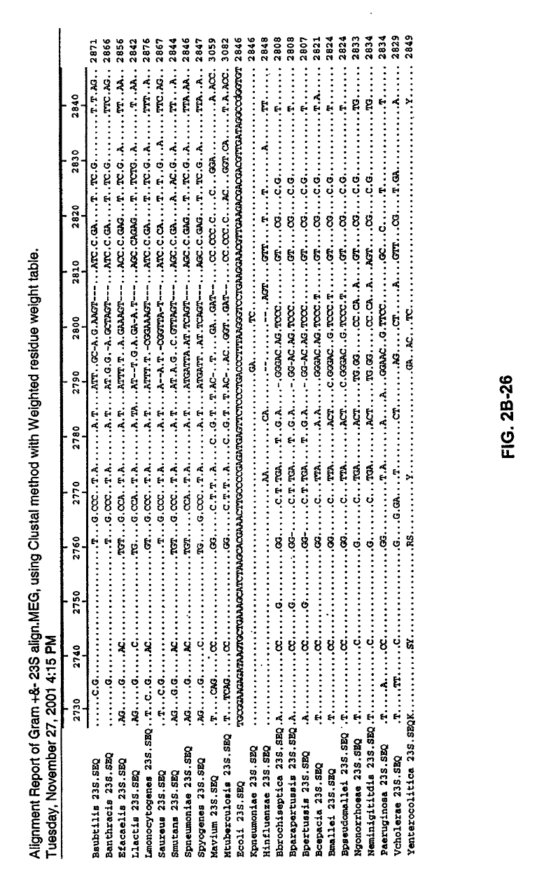

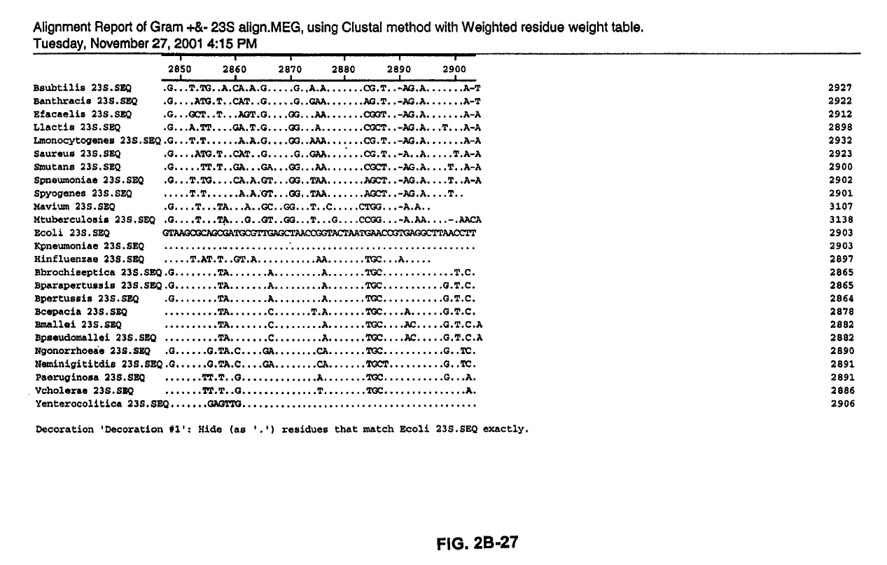

Targeting regions for prokaryotic 16S and 23S rRNAs were designed based on a sequence comparison of different rRNAs from different bacteria to E. coli rRNA with MegAlign sequence analysis software version 4.05 from DNA Star, Incorporated (FIG. 2). The targeting regions are shown, in the examples below, 3′ of the bridging regions. Thus, the targeting region encompasses the remaining, non-bridging region of each molecule described below. SEQ ID NOs are provided for the targeting regions of the bridging nucleic acids provided below (i.e., sequence of bridging regions not included in SEQ ID NO.).

16S prokaryotic rRNA bridging oligonucleotides

| | |
|---|---|
| d16S-358 | (SEQ ID NO:1) |

5′-AAAAAAAAAAAAAAAAAAACTGCTGCCTCCCGTAGGAGTCT-3′

| | |
|---|---|
| d16S-537 | (SEQ ID NO:2) |

5′-AAAAAAAAAAAAAAAAAACGTATTACCGCGGCTGCTGGCAC-3′

| | |
|---|---|
| d16S-548 | (SEQ ID NO:3) |

5′-AAAAAAAAAAAAAAAAAACGCCCAGTAATTCCGATTAACGC-3′

| | |
|---|---|
| d16S-807 | (SEQ ID NO:4) |

5′-AAAAAAAAAAAAAAAAAATGGACTACCAGGGTATCTAATCC-3′

| | |
|---|---|
| d16S-1092 | (SEQ ID NO:5) |

5′-AAAAAAAAAAAAAAAAAAGGGTTGCGCTCGTTGCGGGACTT-3′

| | |
|---|---|
| d16S-3′ | (SEQ ID NO:6) |

5′-AAAAAAAAAAAAAAAAAATAAGGAGGTGATCCAACCGCAGG-3′

23S prokaryotic rRNA bridging oligonucleotides

| | |
|---|---|
| d23S-488 | (SEQ ID NO:7) |

5′-AAAAAAAAAAAAAAAAAAGGTTCTTTTTCACTCCCCTCGCC-3′

| | |
|---|---|
| d23S-581 | (SEQ ID NO:8) |

5′-AAAAAAAAAAAAAAAAAAGACCCATTATACAAAAGGTACGC-3′

| | |
|---|---|
| d23S-1118 | (SEQ ID NO:9) |

5′-AAAAAAAAAAAAAAAAAAGCCCCGTTACATCTTCCGCGCAG-3′

| | |
|---|---|
| d23S-1926 | (SEQ ID NO:10) |

5′-AAAAAAAAAAAAAAAAAACGACAAGGAATTTCGCTACCTTA-3′

| | |
|---|---|
| d23S-1954 | (SEQ ID NO:11) |

5′-AAAAAAAAAAAAAAAAAAACTTACCCGACAAGGAATTTCGC-3′

| | |
|---|---|
| d23S-2511 | (SEQ ID NO:12) |

5′-AAAAAAAAAAAAAAAAAAGAGCCGACATCGAGGTGCCAAAC-3′

| | |
|---|---|
| d23S-3′ | (SEQ ID NO:13) |

5′-AAAAAAAAAAAAAAAAAAAAGGTTAAGCCTCACGGTTCATT-3′

| | |
|---|---|
| d23S-1704 | (SEQ ID NO:15) |

5′-AAAAAAAAAAAAAAAAAACCCCTTCTCCCGAAGTTACGGGG-3′

| | |
|---|---|
| d23S-1105 | (SEQ ID NO:16) |

5′-AAAAAAAAAAAAAAAAAAAGTGAGCTATTACGCTTTCTTT-3′

RNA oligo bridging oligonucleotide

| | |
|---|---|
| r23S-3′ | (SEQ ID NO:14) |

5′-AAAAAAAAAAAAAAAAAAAAAAGGUUAAGCGUCACGGUUCAUU-(inverted
(dT))-3′ (inverted refers to bases attached 3′ to 3′)

Eukaryotic 18S rRNA bridging oligonucleotides

| | |
|---|---|
| d18S-3711 | (SEQ ID NO:17) |

AAA AAA AAA AAA AAA AAA TAC CGG CCG TGC GTA CTT AGA CA

| | |
|---|---|
| d18S-4238 | (SEQ ID NO:18) |

AAA AAA AAA AAA AAA AAA TGC CCT CCA ATG GAT CCT CGT TA

| | |
|---|---|
| d18S-5482 | (SEQ ID NO:19) |

AAA AAA AAA AAA AAA AAA CTA CGG AAA CCT TGT TAC GAC TT

Eukaryotic 28S rRNA bridging oligonucleotides

| | |
|---|---|
| d28S-11599 | (SEQ ID NO:20) |

AAA AAA AAA AAA AAA AAA GAG CAC TGG GCA GAA ATC ACA TC

| | |
|---|---|
| d28S-7979 | (SEQ ID NO:21) |

AAA AAA AAA AAA AAA AAA GTT TCT TTT CCT CCG CTG ACT AA

| | |
|---|---|
| d28S-12533 | (SEQ ID NO:22) |

AAA AAA AAA AAA AAA AAA TCC TCA GCC AAG CAC ATA CAC CA

2. Binding Buffer (also Referred to as Hybridization Buffer)

3 M TMAC, 10 mM Tris, (pH 7.0)

3. Bridging Nucleic Acid Mixture

Mixtures of 16S, 23S, 18S, and/or 28S bridging oligonucleotides were used. All oligonucleotides were purchased from IDT and purified from polyacrylamide gels.

4. Capture Nucleic Acid (Oligo(dT) MagBeads)

Seradyn MGOL #2815-2103.

5. Wash Solution

2 M TMAC, 6.67 mM Tris (pH 7.0) (this is a dilution of binding buffer).

### Example 2

### Methods for rRNA Depletion from Prokaryotic Total RNA

The following methods are provided by way of example for practicing methods of the invention. They have been performed and shown to effect methods of the invention. The invention is not intended to be limited to these protocols, and it is specifically contemplated that variations of the methods below may be employed that fall within the scope of the invention if they effect depletion, isolation, or separation of a targeted nucleic acid, particularly rRNA.

This example demonstrates the depletion of 16S and 23S rRNA from E. coli total RNA.

RNA/Bridging Nucleic Acid Mixture Annealing

RNA (10 μg/15 μl) was added to 200 μl of binding buffer. The bridging nucleic acid mixture consisted of d16S-807 (5 μM), d16S-1092 (5 μM), d23S-1954 (5 μM), d23S-2511 (5 μM). The bridging nucleic acid mixture (4 μl) was added to the RNA and the mixture was incubated at 70° C. for 10 minutes and then shifted to 37° C. for 30 minutes.

Thirty minutes was found to be an adequate time for the annealing step. Longer time periods can be used with no adverse effects. Between fifteen and 120 minutes have been used successfully in the methods of the invention.

Preparation of Capture Nucleic Acid

Capture nucleic acid (Oligo (dT) MagBeads, Seradyn) in storage buffer was mixed and 50 μl was removed to a separate tube. A magnetic stand was applied to the side of the tube to capture the magnetic beads and the supernatant was removed. The capture nucleic acid was equilibrated one time with distilled, deionized water (50 μl) and once with binding buffer (50 μl). The captured nucleic acid was captured again with a magnetic stand, and the binding buffer wash was removed. The magnetic beads were resuspended in 50 μl of binding buffer.

rRNA Capture

Following the 30 minute annealing of RNA with the bridging nucleic acid mixture, the capture nucleic acid was added and the mixture was incubated at room temperature for 15 minutes. A magnetic stand was then applied to the tube to capture the magnetic beads. The supernatant containing mRNA, 5S rRNA, and tRNAs was removed to another tube and saved. An optional washing step was performed next. The magnetic beads were washed with Wash Solution (100 μl) and captured again. The wash supernatant was removed and added to the original supernatant.

Fifteen minutes was found to be an adequate time for rRNA capture. Longer time periods can be used with no adverse effects. rRNA capture likley occurs rapidly, and capture times of 5 minutes—60 minutes have been used successfully in the methods of the invention.

Precipitating mRNA

mRNA, 5S rRNA, and tRNAs were precipitated by adding 1/10 volume of 3M NaOAc (pH 5.5) and 3 volumes of 100% EtOH and incubating at −20° C. for 60 minutes. The precipitated RNA was pelleted in a microfuge, washed with 70% EtOH, and resuspended in TE (pH 8.0).

Analysis of Purified mRNA

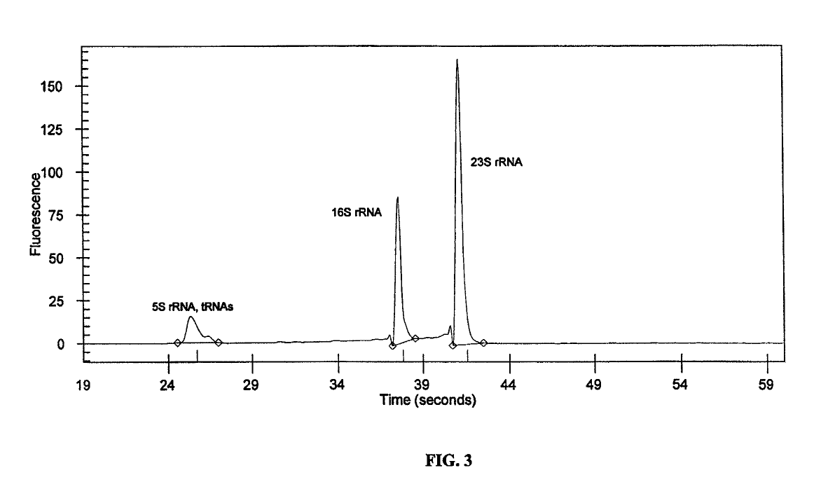

Purified mRNA was analyzed with the Caliper RNA 6000 LabChip kit on an Agilent Bioanalyzer. Purified RNA was compared with a control E. coli total RNA sample that was carried through the reaction as described above, except that the Bridging Nucleic Acid Mixture was left out. This assay system uses electrophoretic and electrokinetic separation in a capillary electrophoresis type system. The rRNAs appear as peaks on an electropherogram (FIG. 3). The percentage of a rRNA present in the sample is calculated form the area under the peak.

Under the protocol conditions described above, the 5S+tRNA peak area is essentially the same in the control and in experimental samples. The % of 16S or 23S rRNA removed was calculated using the ratios of 16S_{peak area}/5Sₚₑₐₖ area and 23S_{peak area}/5S_{peak area}. Enriched and control RNAs with similar 5S+tRNA peak areas were compared.

% 16S rRNA removed=(16S_{peak area}/5S_{peak area})_{no oligos}control−(16S_{peak area}/5S_{peak area)}ₑₓₚₑᵣᵢₘₑₙₜₐₗ X 100

(16S_{peak area/}5Sₚₑₐₖarea)_{no oligos control}

A corresponding formula was used to calculate % 23S rRNA removed.

Electropherograms of RNA from a control reaction and from an experimental reaction after ribosomal RNA depletion are shown in FIG. 3 and FIG. 4.

### Example 3

### Evaluations of Efficacy with Prokaryotic Targets

The materials and methods of Examples 1 and 2 were employed to determine the efficiency of removal of 16S rRNA or 23S rRNA or both from E. coli total RNA. Changes in the parameters of the experiments are noted when appropriate. These experiments were performed to evaluate the efficacy of various bridging nucleic acids and reaction conditions.

The following results are from reactions that employed 10 μg of E. coli total RNA, 40 pmol of total 16S rRNA bridging nucleic acid, 40 pmol of total 23S rRNA bridging nucleic acid, and 50 μl of capture nucleic acid described in Example 1.

| | % 16S Removed | % 23S Removed |
|---|---|---|
| Bridging Nucleic Acid | average of 2 | average of 2 |
| 16S/23S | reactions | reactions |
| d16S-358/d23S-2511 | 96.48285 | 89.86496 |
| d16S-537/d23S-1954 | 97.47974 | 91.32074 |
| d16S-537/d23S-2511 | 97.48704 | 91.216 |
| d16S-807/d23S-1954 | 95.79126 | 89.85388 |
| d16S-807/d23S-2511 | 95.25362 | 91.06399 |
| d16S-1092/d23S-1118 | 97.91265 | 96.50658 |
| d16S-1092/d23S-1954 | 96.7473 | 89.40605 |
| d16S-1092/d23S-2511 | 97.61689 | 91.5964 |
| d16S-358/d23S-1954 | 96.74434 | 88.07242 |
| d16S1092/d23S-1954 | 97.19134 | 98.44728 |
| (20 pmol) | | |
| d23S-2511 (20 pmol) | | |

The following results are from reactions that employed 10 μg of E. coli total RNA, 26 pmol of 16S rRNA bridging nucleic acid, 26 pmol of 23S rRNA bridging nucleic acid, and 35 μl of capture nucleic acid described in Example 1.

| | % 16S Removed | |
|---|---|---|
| Bridging Nucleic Acid | average of 2 | % 23S Removed |
| 16S/23S | reactions | average of 2 reactions |
| d16S-1092/d23S-1118 | 97.38534 | 95.02083 |
| d16S-1092/d23S-1957 | 97.8291 | 90.798 |

The following results are from reactions that employed 10 μg of E. coli total RNA, 75 pmol of 16S rRNA bridging nucleic acid, 75 pmol of 23S rRNA bridging nucleic acid, and 100 μl of capture nucleic acid described in Example 1.

| | % 16S Removed | |
|---|---|---|
| Bridging Nucleic Acid | average of 2 | % 23S Removed |
| 16S . . . 23S | reactions | average of 2 reactions |
| d16S-1092 . . . d23S-1118 | 99.14812 | 99.11895 |
| d16S-1092 . . . d23S-1954 | 98.79938 | 98.45245 |
| d16S-1092 . . . d23S-2511 | 99.00567 | 98.84033 |

The following results are from reactions that employed 10 μg of E. coli total RNA, 37.5 pmol of 16S rRNA bridging nucleic acid, 37.5 pmol of 23S rRNA bridging nucleic acid, and 50 μl of capture nucleic acid described in Example 1.

| Bridging | % 16S Removed | % 23S Removed |
|---|---|---|
| Nucleic Acid | average of 2 | average of 2 |
| 16S/23S | reactions | reactions |
| d16S-1092/d23S-1118 | 98.95563 | 98.28748 |
| d16S-1092/d23S-1954 | 97.83593 | 94.84438 |

The following results are from reactions that employed 10 μg of E. coli total RNA, 75 pmol of 16S rRNA bridging nucleic acid or 75 pmol of 23S rRNA bridging nucleic acid with 75 μl of capture nucleic acid described in Example 1.

| Bridging Nucleic Acid | | |
|---|---|---|
| 16S/23S | % 16S Removed | % 23S Removed |
| n.a./d23S-581 | — | 98.98529 |
| n.a./d23S-581 | — | 98.87251 |
| n.a./d23S-1118 | — | 93.62175 |
| n.a./d23S-1118 | — | 91.4927 |
| n.a./d23S-1954 | — | 98.68262 |
| n.a./d23S-1954 | — | 99.03237 |
| n.a./d23S-2511 | — | 99.31982 |
| n.a./d23S-2511 | — | 99.13291 |
| d16S-358/n.a. | 97.65586 | — |
| d16S-358/n.a. | 97.51393 | — |
| d16S-537/n.a. | 99.16427 | — |
| d16S-537/n.a. | 98.92345 | — |
| d16S-807/n.a. | 98.0661 | — |
| d16S-807/n.a. | 98.14292 | — |

n.a. = not applicable

The following results are from reactions that employed 5 μg of E. coli total RNA, 25 pmol of each 16S rRNA or 23S rRNA bridging nucleic acid, and 25 μl of capture nucleic acid described in Example 1. The rRNA/bridging nucleic acid annealing reaction was for 60 minutes at 37° C.

| Bridging Nucleic Acid | | |
|---|---|---|
| 16S/23S | % 16S Removed | % 23S Removed |
| n.a./d23S-488 | — | ˜100 |
| n.a./d23S-1118 | — | ˜100 |
| d16S-3′/d23S-488 | 89.024 | 94.228 |
| d16S-548/d23S-488 | ˜100 | 93.718 |
| d16S-1092/d23S-488 | ˜100 | 92.652 |

The following results are from reactions that employed 5 μg of E. coli total RNA, 16S rRNA bridging nucleic acid as indicated, 23S rRNA bridging nucleic acid as indicated, and 25 μl of capture nucleic acid described in Example 1. The rRNA/bridging nucleic acid annealing reaction was for 120 minutes at 37° C.

| Bridging Nucleic Acid | | |
|---|---|---|
| 16S/23S | % 16S Removed | % 23S Removed |
| d16S-3′ (25 pmol)/n.a. | 89.137 | — |
| d16S-548 (25 pmol)/n.a. | ˜100 | — |
| d16S-1092 (25 pmol)/n.a. | ˜100 | — |
| d16S-3′ (25 pmol) | ˜100 | — |
| d16S-548 (25 pmol)/n.a. | | |
| d16S-3′ (25 pmol) | ˜100 | — |
| d16S-1092 (25 pmol)/n.a. | | |
| d16S-548 (25 pmol) | ˜100 | — |
| d16S-1092 (25 pmol)n.a. | | |
| d16S-548 (25 pmol)/ | ˜100 | ˜100 |
| d23S-3′ (25 pmol) | | |
| d16S-1092 (25 pmol)/ | ˜100 | ˜100 |
| d23S-3′ (25 pmol) | | |
| d16S-3′ (25 pmol)/ | 92 | ˜100 |
| d23S-3′ (25 pmol) | | |

### Example 4

### The Effect of Washing the Capture Nucleic Acid

The purpose of this experiment was to determine if washing the capture nucleic acid and combining the wash with the purified mRNA had an effect on the presence of rRNA in the purified mRNA sample. Reactions employed 10 μg of E. coli total RNA, 75 pmol d16S-1092, 75 pmol of d23S-d1118, and 100 μl of capture nucleic acid described in Example 1. The rRNA/bridging nucleic acid annealing reaction proceeded for 60 min at 37° C. After the nucleic acid capture step, the capture nucleic acid (with bound rRNA) was resuspended and washed with 100 μl of the indicated solution at room temperature for 5 minutes. The capture nucleic acid was re-captured with a magnetic stand and the supernatant was removed and combined with mRNA in the supernatant from the first capture. mRNA in the combined supernatants were precipitated with ethanol and evaluated with RNA 6000 Lab Chip assay for the presence of rRNAs. The percent of rRNA removal for the entire process is indicated in the table below.

| Wash | % 16S Removed | % 23S Removed |
|---|---|---|
| 0.4 M TMAC | 66.061 | 66.175 |
| 1.0 M TMAC | 95.810 | 96.708 |
| 1.5 M TMAC | ˜100 | ˜100 |
| 2.0 M TMAC | ˜100 | ˜100 |

These results demonstrate that lowering the molarity of the TMAC wash solution increases the stringency of the rRNA capture reaction when the temperature is held constant at room temperature. The results also demonstrate that washing the capture nucleic acid magnetic beads with 1.5 and 2.0 M TMAC does not remove rRNA from the capture nucleic acid.

### Example 5

### Evaluation of Efficacy with Prokaryotic and Eukaryotic rRNA Targets

The purpose of this example was to evaluate efficacy of the methods of the invention for depleting 16S rRNA, 18S rRNA, 23S rRNA, and 28S rRNA from mixtures of prokaryotic and eukaryotic total RNA. Depletion methods were verified using various mammalian samples, including rat livers.

Equal amounts (2.5 μg) of E. coli total RNA and rat liver total RNA were mixed prior to the mRNA enrichment procedure. The bridging oligonucleotides employed were:

| | |
|---|---|
| d16S-1092 | (10 pmol) |
| d16S-807 | (10 pmol) |
| d23S-1954 | (10 pmol) |
| d23S-2511 | (10 pmol) |
| d18S-3711 | (20 pmol) |
| d28S-11599 | (20 pmol) |

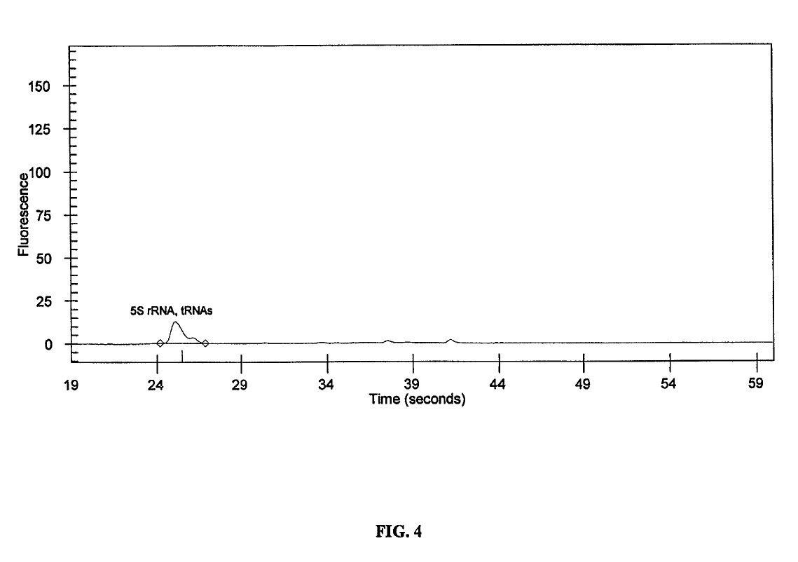

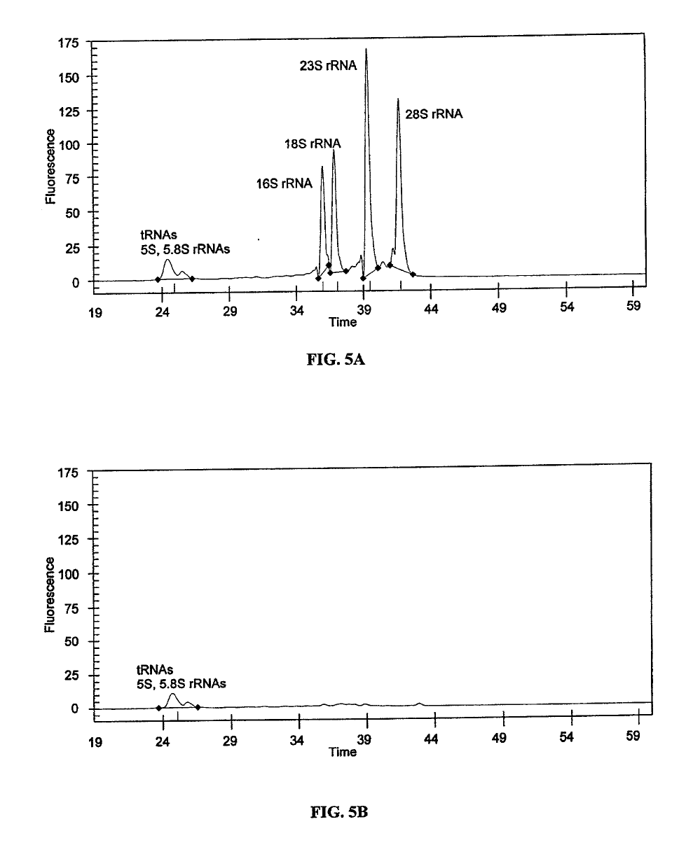

The reaction used 50 μl of capture nucleic acid as described in Example 1. No wash step was employed. Otherwise the reaction was performed according to methods in Example 2. The results are shown in FIGS. 5A and 5B. Note that all rRNAs were depleted except the 5S and 5.8S rRNAs for which no bridging oligonucleotides were added.

### Example 6

### Evaluation of Efficacy with Human rRNA Targets

Additional experiments were done using human samples to evaluate the extent of human rRNA depletion using the bridging oligonucleotides shown below. Depletion of 18S rRNA and 28S rRNA was observed from human liver total RNA. rRNAs were depleted from human liver total RNA (5 μg). The bridging oligonucleotides employed were:

| | |
|---|---|
| d18S-3711 | (40 pmol) |
| d28S-11599 | (40 pmol) |

The reaction used 50 μl of capture nucleic acid as described in Example 1. No wash step was employed. Otherwise the reaction was performed according to Example 2.

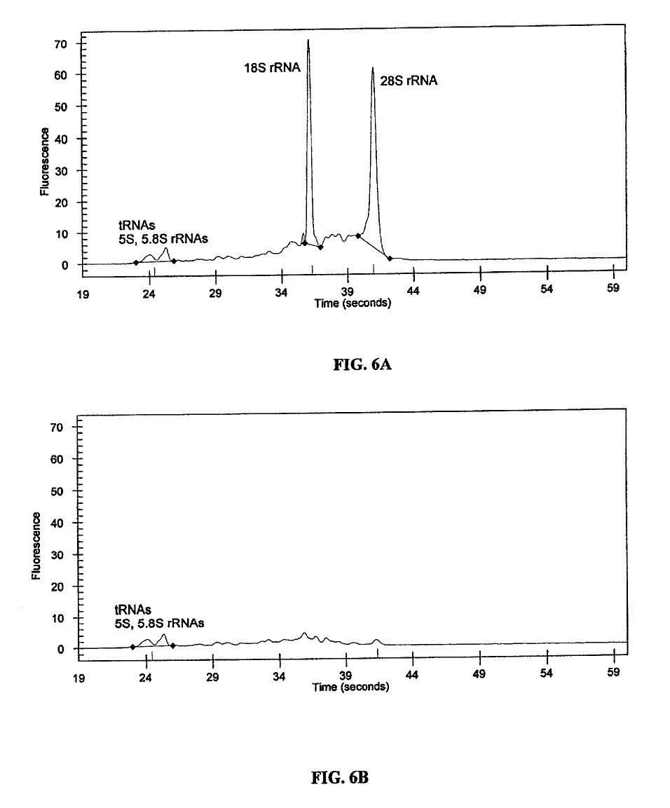

The results are shown in FIGS. 6A and 6B. Note that all rRNAs (18S, 28S) were depleted except the 5S and 5.8S rRNAs for which no bridging oligonucleotides were added.

### Example 7

### Evaluation of Efficacy with Rat rRNA Targets

Additional experiments were done using rat samples to evaluate the extent of rat rRNA depletion using the bridging oligonucleotides shown below. Depletion of 18S rRNA and 28S rRNA was observed from rat liver total RNA. rRNAs were depleted from rat liver total RNA (5 μg). The bridging oligonucleotides employed were:

d18S-3711R-polyA (40 pmol)

d28S-11599R-polyA (40 pmol)

The reaction used 50 μl of capture nucleic acid as described in Example 1. No wash step was employed. Otherwise the reaction was performed according to Example 2.

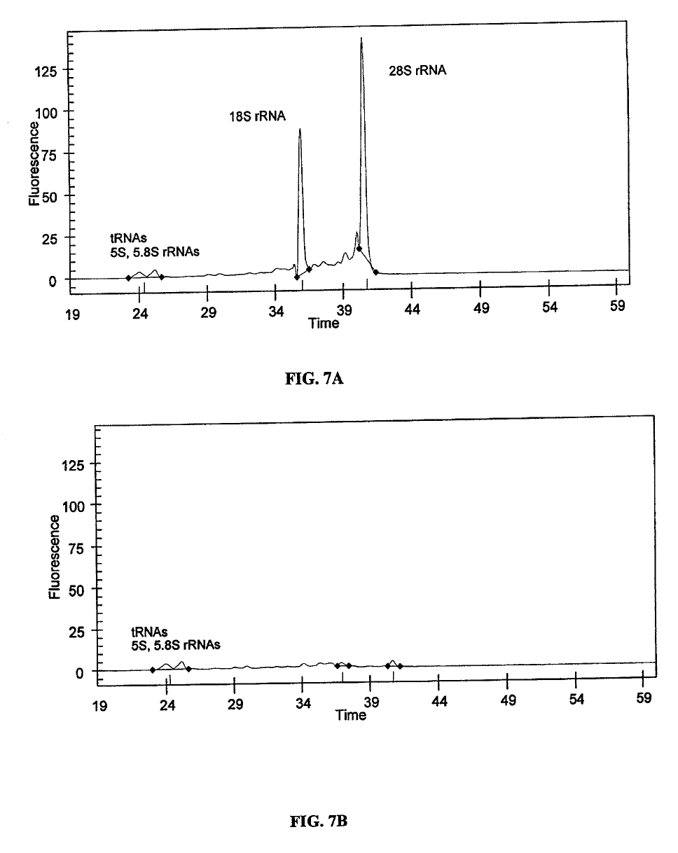

The results are shown in FIGS. 7A and 7B. Note that all rRNAs (18S, 28S) were depleted except the 5S and 5.8S rRNAs for which no bridging oligonucleotides were added.

### Example 8

### Evaluation of Efficacy with Mouse rRNA Targets

Additional experiments were done using mouse samples to evaluate the extent of rat rRNA depletion using the bridging oligonucleotides shown below. Depletion of 18S rRNA and 28S rRNA was observed from mouse liver total RNA (5 μg). The bridging oligonucleotides employed were:

| | |
|---|---|
| d18S-3711R-polyA | (40 pmol) |
| d28S-11599R-polyA | (40 pmol) |

The reaction used 50 μl of capture nucleic acid as described in Example 1. No wash step was employed. Otherwise the reaction was performed according to Example 2.

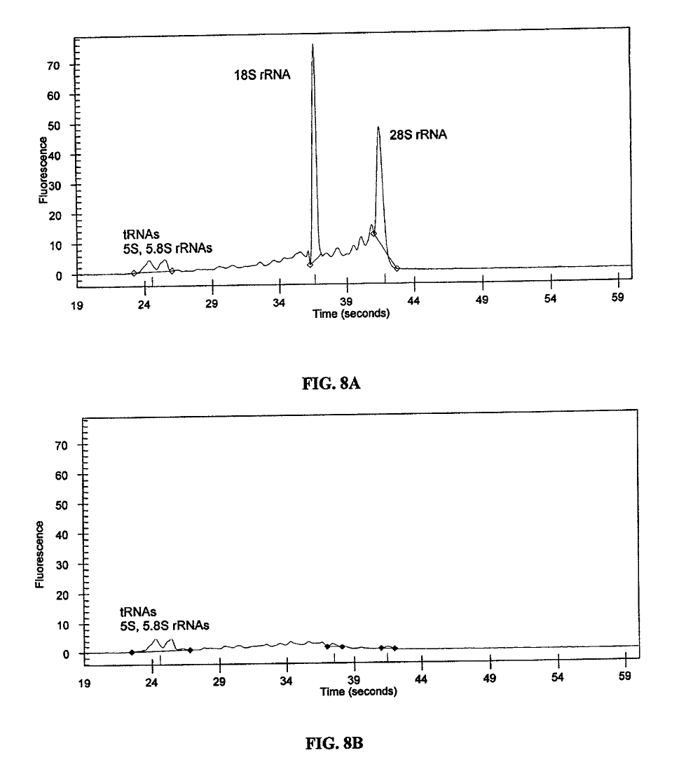

The results are shown in FIGS. 8A and 8B. Note that all rRNAs (18S, 28S) were depleted except the 5S and 5.8S rRNAs for which no bridging oligonucleotides were added.

### Example 9

### Use of Purified E. coli mRNA in Gene Array Expression Analysis

mRNA was purified from total E. coli RNA (10 μg) using the methods of the invention as described in Example 2. A control reaction was also performed in which the bridging nucleic acid mixture was omitted form the reaction. Control total RNA and purified mRNA (1.5 μg) were added to 70 pmol random hexamers in a final volume of 7.25 μl. The mixture was heated at 70° C. for 10 minutes, then transferred to ice for 3 minutes. The following components were added to each reaction:

| | | |
|---|---|---|
| 5 | μl | cDNA 1^{st} strand synthesis buffer (Invitrogen) |
| 2.5 | μl | 0.1 M DTT |
| 1.25 | μl | 10 mM dATP |
| 1.25 | μl | 10 mM dGTP |
| 1.25 | μl | 10 mM dTTP |
| 5 | μl | 10 mCi/ml ³³P-dCTP (Perkin Elmer-NEN) |
| 1 | μl | Superscript II reverse transcriptase (Invitrogen) 200 U/μl |

The reactions were incubated at 42° C. for 120 minutes. Unincorporated nucleotides were removed from the reactions with a Qiaquick PCR cleanup column (Qiagen). The labeled cDNAs (3×10⁷ cpm/blot) were used to probe replicate portions of Panorama™ E. coli gene arrays, using hybridization buffers supplied by the array manufacturer (Sigma-Genosys). The arrays were washed and exposed to film. This example demonstrates a dramatic increase in hybridization signal (sensitivity) on gene arrays when labeled cDNA is prepared from bacterial mRNA, purified according to the methods of the invention, rather than from total RNA.

### Example 10

### Instructions for Use with Kit

The following instructions have been followed with a kit of the invention described below for the successful depletion of 16S and 23S rRNA from a sample comprising prokaryotic RNA populations. Bridging oligonucleotides with targeting regions complementary to 18S and 28S rRNA may be employed according to the method below to effect a similar result (as in Examples 5-8).

Materials Provided with a Kit Embodiment

| | | |
|---|---|---|
| 30 | μl | Control RNA |
| 1.2 | ml | Capture Nucleic Acid [as in Example 1] |
| 7 | ml | Binding Buffer [as in Example 1] |
| 95 | μl | Bridging Oligonucleotide Mix [as in Example 2] |
| 2.4 | ml | Wash Solution [as in Example 1] |
| 1.75 | ml | Nuclease-free Water |
| 50 | ea | RNase-free 1.5 ml tubes |
| 25 | ea | RNase-free 2 ml Elution tubes |
| 200 | μl | Glycogen (5 mg/ml) |
| 875 | μl | 3 M NaOAc |

Experimental Parameters

A. RNA Source

This mRNA enrichment procedure is designed to work with purified total RNA from many different bacteria, including both gram-positive and gram-negative species. The procedure was optimized with total E. coli RNA and has been found to remove 90-99% of the rRNA from Bacillus subtilis, Staphylococcus aureus, Prochlorococcus sp., Neisseria meningitidis, and Pseudomonas aeruginosa, for example. It is contemplated that any eubacterial species may be targeted using the methods and compositions of the invention.

This procedure is designed so that small RNAs (including tRNA and 5S rRNA) remain in the enriched mRNA population. However, if the loss of very small RNA species (<200 base) will not be an issue, the initial isolation of total RNA should be performed with Ambion's RNAQUEOUS KIT. The RNAQUEOUS KIT will remove most small RNA species and provide the highest possible level of mRNA enrichment. If small RNAs are of interest to the user, it is best to avoid glass fiber filter-based purification.

B. Precipitate RNA to Remove Salt and Concentrate if Necessary

Total RNA prepared from a solid-phase extraction method such as RNAQUEOUS can be used immediately after elution because such samples are unlikely to have high levels of salt. On the other hand, RNA isolated by methods that include organic extractions, for example using the products RNAWIZ, TRIZOL or ToTALLY RNA, may have a substantial amount of residual salt. If RNA from these types of procedures has been precipitated only a single time, we recommend doing a second alcohol precipitation and 70% EtOH wash to remove residual salt before starting the enrichment procedure.

The recommended maximum amount of RNA per reaction is 10 μg and the recommended maximum volume for the RNA is 15 μl. If the RNA sample is too dilute, it will be necessary to precipitate and concentrate the RNA to at least 10 μg/15 μl. Precipitate the RNA with:

0.1 volume 5 M Ammonium Acetate or 3 M sodium acetate

1 μl Glycogen (The glycogen acts as a carrier to increase precipitation efficiency from dilute RNA solutions; it is unnecessary for solutions with 200 μg RNA/ml)

2.5 volumes 100% ethanol

a. Leave the precipitation mixture at −20° C. overnight, or quick-freeze it in either ethanol and dry ice, or in a −70° C. freezer for 30 minutes.

b. Recover the RNA by centrifugation at 12,000×g for 30 minutes at 4° C.

c. Carefully remove and discard the supernatant. The RNA pellet may not adhere tightly to the walls of the tubes, so we suggest removing the supernatant by gentle aspiration with a fine-tipped pipette.

d. Centrifuge the tube briefly a second time, and aspirate any additional fluid that collects with a fine-tipped pipette.

e. Add 1 ml 70% ethanol, and vortex the tube a few times. Repellet the RNA by microcentrifuging, for 10 minutes at 4° C. Remove supernatant carefully as in steps c and d above.

RNA should be dissolved in TE or Ambion's THE RNA STORAGE SOLUTION. It is important to accurately quantitate RNA so as not to overload the system. Ambion recommends using the RiboGreen RNA Quantitation Assay and Kit (Molecular Probes) or a high quality, calibrated spectrophotometer.

C. Save an Aliquot of Your Total RNA

If possible, retain a small aliquot (˜1-2 μg) of the total RNA used for comparison with enriched mRNA by gel electrophoresis after the procedure is finished.

Instructions

A. Anneal RNA and Bridging Oligonucleotide Mix

1. Add RNA to Binding Buffer

Add total RNA (up to 10 μg total RNA in a maximum volume of 15 μl) to 200 μl Binding Buffer in a 1.5 ml tube provided with the kit. Close the tube and tap or vortex gently to mix.

2. Add Bridging Oligonucleotide Mix to RNA

Add 4.0 μl of the Bridging Oligonucleotide Mix to the RNA in Binding Buffer. Close the tube and tap or vortex gently to mix. Pulse in a microcentrifuge very briefly to get mixture to bottom of tube.

3. Incubate Reactions at 70° C. for 10 Minutes.

Incubating the mixture at 70° C. for 10 minutes denatures secondary structures in RNA, including the 16S and 23S rRNAs, allowing for maximal hybridization of the bridging oligonucleotides to the rRNAs.

4. Incubate Reactions at 37° C. for 1 Hour.

Incubating the mixture at 37° C. for 1 hour allows for binding of the bridging oligonucleotides to the 16S and 23S rRNA. The Binding Buffer has been optimized to function specifically and efficiently at this temperature.

B. Prepare the Capture Nucleic Acid

During the 1 hour RNA/^{Bridging} Oligonucleotide Mix annealing step, prepare the Capture Nucleic Acid. The Capture Nucleic Acid is in a 1% (10 mg/ml) suspension, vortex the tube briefly before pipetting to be sure they are well suspended.

1. Aliquot the Capture Nucleic Acid

For each 10 μg reaction remove 50 μl Capture Oligos to a 1.5 ml tube. Capture Nucleic Acid for up to 10 reactions can be processed in a single 1.5 ml tube.

2. Wash the Capture Nucleic Acid Once with Water and Once with Binding Buffer

a. Capture the beads (Capture Nucleic Acid) by placing the tube on the Magnetic Stand. Leave the tube on the stand until all of the Capture Nucleic Acid is arranged inside the tube near the magnet. This will take ˜3 minutes for microfuge tubes.

b. Carefully remove the supernatant by aspiration, leaving the beads in the tube, and discard the supernatant.

c. Add Nuclease Free Water to the captured beads at a ratio of 50 μl/50 μl beads).

d. Remove the tube from the Magnetic Stand, resuspend the beads by gently vortexing briefly, recapture the beads with a Magnetic Stand, carefully aspirate the supernatant, leaving the beads in the tube, and discard the supernatant.

e. Add Binding Buffer to the captured beads at a ratio of 50 μl/50 μl beads).

f. Repeat step d.

3. Resuspend the Capture Nucleic Acid in Binding Buffer

a. Add Binding Buffer to the captured beads at a ratio of 50 μl/50 μl beads).

b. Remove the tube from the Magnetic Stand, resuspend the beads by gently tapping the tube or very gentle vortexing.

c. Pulse spin in a microcentrifuge to get liquid to the bottom of the tube.

C. Capture the rRNA with Capture Nucleic Acid and Recover the Enriched mRNA

1. Add Capture Nucleic Acid (50 μl/rxn) to RNA/Bridging Oligonucleotide Mix and Incubate at RT for 15 Minutes.

a After the 1 hour incubation at 37° C. (Step A.4) remove tubes to room temperature (RT) and immediately add 50 μl of the washed and equilibrated beads (Capture Nucleic Acid, from Step B.3c) to each purification reaction. Very gently vortex or tap tube to mix briefly and pulse spin in a microcentrifuge to get liquid to the bottom of the tube.

b. Incubate 15 minutes at RT. During this step the oligonucleotide sequence on the Capture Nucleic Acid anneals to the bridging oligonucleotides. The bridging oligonucleotides remain hybridized to the 16S and 23S rRNAs. The hybridization “sandwich” of bridging oligonucleotide and capture oligonucleotide (via the capture region on the capture oligo and the bridging region on the bridging oligo) is formed at this step.

2. Recover the Supernatant Containing the Enriched mRNA.

a. Capture the beads by placing the tube on the Magnetic Stand. Leave the tube on the stand until all of the beads are arranged inside the tube near the magnet. This will take ˜3 minutes for microfuge tubes. Allow the beads to be completely captured by the magnet for at least 3 minutes.

b. Remove the supernatant by aspiration, being careful not to dislodge the beads. Put the supernatant into a 2 ml nipple bottom tube on ice and save. Do not be overly concerned if there seems to be beads in the removed supernatant. The excess can be removed at the end of the procedure. The supernatant contains the enriched mRNA sample.

3. Wash the Oligo MagBeads with Wash Solution and Recover the Wash.

a. Add Wash Solution to the captured beads at a ratio of 100 μl Wash Solution/50 μl beads.

b. Remove the tube from the Magnetic Stand, resuspend the beads by gently vortexing briefly.

c. Incubate at RT for 5 minutes.

d. Recapture the beads with the Magnetic Stand as in step C.2a. Allow the beads to be completely captured by the magnet for at least 3 minutes.

e. Remove the supernatant by aspiration, being careful not to dislodge the beads. Put this supernatant in the 2 ml nipple bottom tube on ice with that from step C.2b.

D. Precipitate and Resuspend the Enriched mRNA in the Supernatant.

1. Perform an EtOH Precipitation on the Collected Supernatant.

a. Add 1/10 Volume 3M NaOAc (35 μl) and 5 mg/ml glycogen to a final concentration of 100 μg/ml (7 μl) to the supernatant from step C.3.e. (the supernatant volume should be ˜350 μl).

b. Briefly vortex the sample to mix.

c. Add 3 Vol. ice cold 100% EtOH (1175 μl) and mix well by vortexing the sample.

d. Precipitate the sample at −20° C. for at least 1 hour.

e. Centrifuge the sample for 30 min.@13,000 rpm.

f. Carefully decant the supernatant.

g. Add 750 ml ice cold 70% EtOH, vortex briefly, and centrifuge for 5 min. ® 13,000 rpm. Decant the supernatant.

h. Repeat step D.1.g.

i. After decanting the supernatant spin briefly to collect. Remove the remaining supernatant with a pipettor, being careful not to dislodge the pellet. Air dry for 5 min.

2. Resuspend the Enriched mRNA in an Appropriate Buffer.

a. After the pellet has air dried for no more than 5 min. add 2 μl TE pH 8.0 (RNA STORAGE SOLUTION, 1 mM EDTA or Nuclease-Free ddH₂O could be substituted).

b. Allow the RNA to resuspend for 15 min. at room temperature. Vortex the sample vigorously to resuspend. Collect the sample by brief centrifugation. NOTE: If the pellet refuses to go into solution the sample can be incubated for 5 min. @ 70° C. This should help resuspend the pellet. NOTE: Often there will be beads remaining in the sample after the precipitation (This will cause the RNA solution to appear brownish in color). This can be remedied by applying the sample to the Magnetic stand for ˜3 min. and removing the supernatant to a new tube.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. application Ser. No. 09/854,412

U.S. Pat. No. 4,486,539

U.S. Pat. No. 4,563,419

U.S. Pat. No. 4,659,774

U.S. Pat. No. 4,682,195

U.S. Pat. No. 4,683,202

U.S. Pat. No. 4,751,177

U.S. Pat. No. 4,816,571

U.S. Pat. No. 4,868,105

U.S. Pat. No. 4,894,325

U.S. Pat. No. 4,959,463

U.S. Pat. No. 5,124,246

U.S. Pat. No. 5,141,813

U.S. Pat. No. 5,200,314

U.S. Pat. No. 5,214,136

U.S. Pat. No. 5,216,141

U.S. Pat. No. 5,223,618

U.S. Pat. No. 5,264,566

U.S. Pat. No. 5,273,882

U.S. Pat. No. 5,288,609

U.S. Pat. No. 5,378,825

U.S. Pat. No. 5,412,087

U.S. Pat. No. 5,428,148

U.S. Pat. No. 5,432,272

U.S. Pat. No. 5,445,934

U.S. Pat. No. 5,446,137

U.S. Pat. No. 5,457,025

U.S. Pat. No. 5,466,786

U.S. Pat. No. 5,470,967

U.S. Pat. No. 5,500,356

U.S. Pat. No. 5,539,082

U.S. Pat. No. 5,554,744

U.S. Pat. No. 5,574,146

U.S. Pat. No. 5,589,335

U.S. Pat. No. 5,602,240

U.S. Pat. No. 5,602,244

U.S. Pat. No. 5,610,289

U.S. Pat. No. 5,614,617

U.S. Pat. No. 5,623,070

U.S. Pat. No. 5,645,897

U.S. Pat. No. 5,652,099

U.S. Pat. No. 5,670,663

U.S. Pat. No. 5,672,697

U.S. Pat. No. 5,681,947

U.S. Pat. No. 5,700,922

U.S. Pat. No. 5,702,896

U.S. Pat. No. 5,708,154

U.S. Pat. No. 5,709,629

U.S. Pat. No. 5,714,324

U.S. Pat. No. 5,714,331

U.S. Pat. No. 5,714,606

U.S. Pat. No. 5,719,262

U.S. Pat. No. 5,723,597

U.S. Pat. No. 5,736,336

U.S. Pat. No. 5,744,305

U.S. Pat. No. 5,759,777

U.S. Pat. No. 5,763,167

U.S. Pat. No. 5,766,855

U.S. Pat. No. 5,773,571

U.S. Pat. No. 5,777,092

U.S. Pat. No. 5,786,461

U.S. Pat. No. 5,792,847

U.S. Pat. No. 5,858,988

U.S. Pat. No. 5,859,221

U.S. Pat. No. 5,872,232

U.S. Pat. No. 5,886,165

U.S. Pat. No. 5,891,625

U.S. Pat. No. 5,897,783

U.S. Pat. No. 5,908,845

U.S. Pat. No. 5,945,525

U.S. Pat. No. 6,001,983

U.S. Pat. No. 6,013,440

U.S. Pat. No. 6,037,120

U.S. Pat. No. 6,060,246

U.S. Pat. No. 6,090,548

U.S. Pat. No. 6,110,678

U.S. Pat. No. 6,140,496

U.S. Pat. No. 6,203,978

U.S. Pat. No. 6,221,581

U.S. Pat. No. 6,228,580

U.S. Pat. No. 6,309,823

U.S. Pat. No. 6,316,193

U.S. Pat. No. 6,322,971

U.S. Pat. No. 6,324,479

U.S. Pat. No. 6,329,140

U.S. Pat. No. 6,329,209

EP 266,032

PCT/EP/01219

PCT/U.S.00/29865

WO 01/32672

WO 86/05815

WO90/06045

WO 92/20702

The entire issue of Current Opinion in Microbiology, Volume 4, February 2001.

Amara et al., Nucl. Acids Res. 25:3465-3470, 1997.

Arfin et al., J. Biol. Chem. 275:29672-29684.

Ausubel et al., In: Current Protocols in Molecular Biology, John, Wiley & Sons, Inc, New York, 1994.

Beaucage, Methods Mol. Biol 20:33-61, 1993.

Chuang et al., J. Bacteriol. 175:2026-2036, 1993.

Coombes et al., Infect. Immun. 69:1420-1427, 2001.

Comelis et al., Curr. Opin. Microbiol. 4:13-15, 2001.

Cummings et al., Emerg. Inf. Dis. 6:513-524, 2000.

DeRisi et al., Nature Genetics 14:457-460, 1996.

Detweller et al., Proc. Natl. Acad. Sci. USA 98:5850-5855, 2001.

Egholm et al., Nature 365(6446):566-568, 1993.

Feng et al., Proc. Natl. Acad. Sci. USA 97:6415-6420, 2000.

Fox, J. L. et al., ASM News 67:247-252, 2001.

Froehler et al., Nucleic Acids Res., 14(13):5399-5407, 1986.

Gillam et al., J. Biol. Chem. 253(8):2532-9, 1978.

Gillam et al., Gene 8(1):99-106, 1979.

Gingeras et al., ASM News 66:463-469, 2000.

Graham et al, Curr. Opin. Microbiol. 4:65-70, 2001.

Graham et al., Proc. Natl. Acad. Sci. USA 96; 11554-11559, 1999.

Ichikawa et al., Proc. Natl. Acad. Sci. USA 97:9659-9664, 2000.

Itakura et al., J. Am. Chem. Soc. 97(25):7327-32, 1975.

Kagnoff et al., Curr. Opin. Microbiol. 4:246-250, 2001.

Khorana, Science 203(4381):614-25, 1979.

Klug et al., Methods Enzymol. 152:316-325, 1987.

Koshkin et al, Tetrahedron 54:3607-3630, 1998.

Koshkin et al., J. Am. Chem. Soc. 120:13252-13253, 1998.

Kricka, Nonisotopic DNA Probe Techniques, Academic Press, San Diego, Calif., 1992.

Liang et al., Methods Enzymol. 254:304-321, 1995.

Lockhart et al., Nature Biotech. 14:1675, 1996.

Maskos et al., Nuc. Acids. Res. 20:1679-1684, 1992.

Neidhardt et al., in Escherichia coli and Salmonella (Neidhardt, F C, Ed.), Vol. 1, pp.13-16, ASM Press, Washington, D.C., 1996.

Newton et al., J Comput. Biol. 8:37-52, 2001.

Pietu et al., Genome Res. 6:492, 1996.

Plum, et al., Infect. Immun. 62:476-483, 1994.

Rappuoli, R. Proc. Natl. Acad. Sci. USA 97:13467-13469, 2000.

Robinson et al., Gene 148:137-141, 1994.

Rosenberger et al., J. Immunol. 164:5894-5904, 2000.

Sambrook et. al., In: Molecular Cloning: A Laboratory Manual, 2d Ed., Cold Spring Harbor Laboratory Press, Cold Spring Harbor, N.Y., 1989.

Sambrook et al., In: Molecular Cloning: A Laboratory Manual, 3rd Ed., Cold Spring Harbor Press, Cold Spring Harbor, N.Y., 2001.

Schena et al., Science 270:467-470, 1995a.

Schena et al., Proc. Natl. Acad. Sci. USA 93:10539-11286, 1995b.

Shalon et al., Genome Res. 6:639-645, 1996.

Su et al., Molec. Biotechnol. 10:83-85, 1998.

Velculescu et al., Science 270:484-487, 1995.

Wahlestedt et al., PNAS 97:5633-5638, 2000.

Wei et al., J. Bacteriol. 183:545-556, 2001.

Wendisch, et al., Anal. Biochem. 290:205-213, 2001.

Wood et al., Proc. Natl. Acad. Sci. USA. 82:1585-1588, 1985.

Yoshida et al., Nucl. Acids Res. 29:683-692, 2001.

Zhao et al., Gene 156:207, 1995.

What is claimed is:

1. A method for depleting or isolating a targeted nucleic acid from a sample comprising:
a) incubating the sample with a first bridging oligonucleotide comprising (1) at least one bridging region comprising at least 5 nucleic acid residues and (2) at least one targeting region comprising at least 5 nucleic acid residues, under conditions allowing hybridization between the first targeting region and the targeted nucleic acid;
b) incubating the first bridging oligonucleotide with a capture oligonucleotide comprising a nonreacting structure and a capture region comprising at least 5 nucleic acid residues, under conditions allowing hybridization between the bridging region and the capture region; and
c) isolating the targeted nucleic acid from the remainder of the sample.

2. The method of claim 1 wherein the targeted nucleic acid is rRNA.

3. The method of claim 2, wherein the rRNA is prokaryotic 16S, prokaryotic 23S, eukaryotic 17S or 18S, or eukaryotic 28S rRNA.

4. The method of claim 3, wherein the rRNA comprises the sequence of SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:71, SEQ ID NO:72, or SEQ ID NO:73.

5. The method of claim 1, wherein the sample comprises eukaryotic nucleic acid.

6. The method of claim 1, wherein the sample comprises prokaryotic nucleic acid.

7. The method of claim 6, wherein the prokaryotic nucleic acid is from a gram positive bacterium.

8. The method of claim 6, wherein the prokaryotic nucleic acid is from a gram negative bacterium.

9. The method of claim 1, wherein the bridging region, targeting region, or capture region comprises at least 10 nucleic acid residues.

10. The method of claim 9, wherein the bridging region, targeting region, or capture region comprises at least 15 nucleic acid residues.

11. The method of claim 10, wherein the bridging region, targeting region, or capture region comprises at least 20 nucleic acid residues.

12. The method of claim 1, wherein the bridging region or the capture region is polypurine or polypyrimidine.

13. The method of claim 12, wherein the bridging region is polypurine and the capture region is polypyrimidine.

14. The method of claim 1, further comprising incubating the sample with a second bridging oligonucleotide comprising (1) at least one bridging region comprising at least 5 nucleic acid residues and (2) at least one targeting region comprising at least 5 nucleic acid residues, under conditions allowing hybridization between the targeting region of the second bridging oligonucleotide and the targeted nucleic acid.

15. The method of claim 14, wherein the targeting region of the first bridging oligonucleotide is complementary to the sequence of a targeted nucleic acid and the targeting region of the second bridging oligonucleotide is complementary to a different sequence of a targeted nucleic acid.

16. The method of claim 15, wherein the targeting region of the first bridging oligonucleotide and the targeting region of the second bridging oligonucleotide are complementary to the same targeted nucleic acid.

17. The method of claim 15, wherein the targeting region of the first bridging oligonucleotide and the targeting region of the second bridging oligonucleotide are complementary to different targeted nucleic acids.

18. The method of claim 17, wherein the targeting region of the first bridging oligonucleotide is complementary to a sequence of the largest rRNA molecule and the targeting region of the second bridging oligonucleotide is complementary to a sequence of the second largest rRNA molecule in the sample.

19. The method of claim 14, wherein the targeting region of the first or second bridging oligonucleotide hybridizes to a sequence located between 100 and 5000 residues of the 5′ or 3′ end of the targeted nucleic acid.

20. The method of claim 19, wherein the targeting region of the first or second bridging oligonucleotide hybridizes to a sequence located between 150 and 4000 residues of the 5′ or 3′ end of the targeted nucleic acid.

21. The method of claim 20, wherein the targeting region of the first or second bridging oligonucleotide hybridizes to a sequence located between 200 and 3000 residues of the 5′ or 3′ end of the targeted nucleic acid.

22. The method of claim 21, wherein the targeting region of the first or second bridging oligonucleotide hybridizes to a sequence located between 250 and 2000 residues of the 5′ or 3′ end of the targeted nucleic acid.

23. The method of claim 22, wherein the targeting region of the first or second bridging oligonucleotide hybridizes to a sequence located between 300 and 1500 residues of the 5′ or 3′ end of the targeted nucleic acid.

24. The method of claim 23, wherein the targeting region of the first or second bridging oligonucleotide hybridizes to a sequence located between 350 and 1000 residues of the 5′ or 3′ end of the targeted nucleic acid.

25. The method of claim 24, wherein targeting region of the first or second bridging oligonucleotide hybridizes to a sequence located between 400 and 900 residues of the 5′ or 3′ end of the targeted nucleic acid.

26. The method of claim 25, wherein the targeting region of the first or second bridging oligonucleotide hybridizes to a sequence located between 450 and 800 residues of the 5′ or 3′ end of the targeted nucleic acid.

27. The method of claim 26, wherein the targeting region of the first or second bridging oligonucleotide hybridizes to a sequence located between 500 and 700 residues of the 5′ or 3′ end of the targeted nucleic acid.

28. The method of claim 14, wherein the targeting region of the first or second bridging oligonucleotide hybridizes to a sequence at the 3′ or 5′ end of the targeted nucleic acid.

29. The method of claim 14, wherein the targeting region of the first or second bridging oligonucleotide hybridizes to a sequence not within 100 residues from the 3′ or 5′ end of the targeted nucleic acid.

30. The method of claim 14, wherein targeting region of the first or second bridging oligonucleotide hybridizes to a sequence not within 200 residues from the 3′ or 5′ end of the targeted nucleic acid.

31. The method of claim 14, wherein the targeting region of the first or second bridging oligonucleotide hybridizes to a sequence not within 400 residues from the 3′ or 5′ ends of the targeted nucleic acid.

32. The method of claim 14, wherein the targeting region of the first or second bridging oligonucleotide comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

33. The method of claim 1, wherein the bridging oligonucleotide comprises a second targeting region comprising at least 5 nucleic acid residues complementary to a different sequence than the sequence to which the first targeting region is complementary.

34. The method of claim 33, wherein the first targeting region is complementary to a different targeting nucleic acid than the second targeting region is.

35. The method of claim 1, wherein the first bridging oligonucleotide comprises two bridging regions.

36. The method of claim 1, wherein the bridging oligonucleotide or the capture oligonucleotide is RNA, DNA, LNA, iso-bases, or a peptide nucleic acid.

37. The method of claim 1, further comprising washing the capture oligonucleotide after incubation with the sample and the bridging oligonucleotide.

38. The method of claim 1, wherein a) and b) are performed at the same temperature.

39. The method of claim 1, wherein a) and b) are performed at a different temperature.

40. The method of claim 38, wherein a) and b) are performed at the same time.

41. The method of claim 1, wherein the nonreacting structure comprises a bead comprising plastic, glass, teflon, silica, a magnet, cellulose, latex, polystyrene, nylon, cellulose, nitrocellulose, polymethacrylate, polyvinylchloride, or styrene-divinylbenzene

42. The method of claim 41, wherein isolating the targeted nucleic acid away from the sample comprises exposing the sample with the capture oligonucleotide to a magnetic field.

43. The method of claim 1, wherein the nonreacting structure is cellulose.

44. The method of claim 1, wherein the nonreacting structure is biotin.

45. The method of claim 44, wherein isolating the targeted nucleic acid comprises incubating the sample with streptavidin or avidin.

46. The method of claim 1, wherein the sample, capture oligonucleotide, and bridging oligonucleotide are incubated in a buffer comprising TMAC or TEAC.

47. The method of claim 1, further comprising:
d) discarding the portion of the sample that hybridizes to the capture oligonucleotide.

48. The method of claim 2, further comprising:
d) discarding the targeted rRNA nucleic acid; and
e) producing cDNA using mRNA in the remainder of the sample.

49. The method of claim 48, further comprising:
f) attaching the cDNA to a solid support, wherein a nucleic acid array is created.

50. The method of claim 49, wherein the solid support is plastic, glass, or nylon.

51. The method of claim 50, wherein the solid support is a plate.

52. The method of claim 51, wherein the plate is a multiple-well plate.

53. The method of claim 48, further comprising:
f) contacting a nucleic acid array with the cDNA.

54. A method for depleting rRNA from a sample comprising:
a) incubating the sample with at least a first (1) bridging oligonucleotide comprising a bridging region comprising a poly-purine region of at least 5 residues and a targeting region comprising at least 5 contiguous nucleic acid residues complementary to a sequence of an rRNA molecule and a (2) capture oligonucleotide comprising a magnetic bead and a capture region comprising a poly-pyrimidine region of at least 5 residues, under conditions to allow hybridization between the bridging oligonucleotide and the capture oligonucleotide and the bridging oligonucleotide and the rRNA;
b) incubating the sample with a magnetic bead; and
c) isolating the magnetic bead.

55. A kit, in a suitable container means, comprising:
a) a capture oligonucleotide comprising a capture region and a magnetic bead; and
b) at least a first bridging oligonucleotide comprising (1) at least one bridging region complementary to all or part of the capture region of the capture oligonucleotide and a (2) at least one targeting region comprising 10 contiguous nucleic acids complementary to a sequence of an rRNA.

56. The kit of claim 55, wherein the first bridging oligonucleotide comprises a second targeting region.

57. The kit of claim 56, wherein the first and second targeting regions have the same nucleic acid sequence.

58. The kit of claim 56, wherein the first and second targeting regions have different nucleic acid sequences.

59. The kit of claim 58, wherein the first targeting region is complementary to a sequence of an eukaryotic rRNA and the second targeting region is complementary to a sequence of a prokaryotic rRNA.

60. The kit of claim 58, wherein the first targeting region is complementary to a sequence of an eukaryotic rRNA and the second targeting region is complementary to a sequence of a different eukaryotic rRNA than the first targeting region.

61. The kit of claim 58, wherein the first targeting region is complementary to a sequence of a prokaryotic rRNA and the second targeting region is complementary to a sequence of a different prokaryotic rRNA than the first targeting region.

62. The kit of claim 55, further comprising a second bridging oligonucleotide comprising (1) at least one bridging region complementary to all or part of the capture region of the capture oligonucleotide and a (2) at least one targeting region comprising 10 contiguous nucleic acids complementary to a sequence of an rRNA.

63. The kit of claim 62, wherein the targeting region of the second bridging oligonucleotide is complementary to a sequence of the same rRNA as the first targeting region.

64. The kit of claim 62, wherein the targeting region of the first bridging oligonucleotide is complementary to a sequence of the largest rRNA and the targeting region of the second bridging oligonucleotide is complementary to a sequence of the second largest rRNA in the sample.

65. The kit of claim 62, wherein the targeting region of the first bridging oligonucleotide is complementary to a sequence of an eukaryotic rRNA and the targeting region of the bridging oligonucleotide is complementary to a sequence of a prokaryotic rRNA.

66. The kit of claim 64, wherein the targeting region of the first bridging oligonucleotide is complementary to a sequence of an eukaryotic 28S rRNA and the targeting region of the second bridging oligonucleotide is complementary to a sequence of a eukaryotic 17S or 18S rRNA.

67. The kit of claim 64, wherein the targeting region of the first bridging oligonucleotide is complementary to a sequence of a prokaryotic 23S rRNA and the targeting region of the second bridging oligonucleotide is complementary to a sequence of a prokaryotic 16S rRNA.

68. The kit of claim 64, wherein the targeting region of the first bridging oligonucleotide is complementary to a sequence of an eukaryotic 28S rRNA and the targeting region of the second bridging oligonucleotide is complementary to a sequence of a prokaryotic 23S rRNA.

69. The kit of claim 62, further comprising a third bridging oligonucleotide comprising (1) at least one bridging region complementary to all or part of the capture region of the capture oligonucleotide and a (2) at least one targeting region comprising 10 contiguous nucleic acids complementary to a sequence of an rRNA.

70. The kit of claim 69, wherein the targeting region of the third bridging oligonucleotide is complementary to a sequence of a prokaryotic 23S rRNA.

71. The kit of claim 69, wherein the targeting region of the third bridging oligonucleotide is complementary to a sequence of a eukaryotic 18S rRNA.

72. The kit of claim 69, further comprising a fourth bridging oligonucleotide comprising (1) at least one bridging region complementary to all or part of the capture region of the capture oligonucleotide and a (2) at least one targeting region comprising 10 contiguous nucleic acids complementary to a sequence of an rRNA.

73. The kit of claim 72, wherein (i) the targeting region of the first bridging oligonucleotide is complementary to a sequence of a prokaryotic 16S rRNA, (ii) the targeting region of the second bridging oligonucleotide is complementary to a sequence of a prokaryotic 23S rRNA, (iii) the targeting region of the third bridging oligonucleotide is complementary to a sequence of a eukaryotic 18S rRNA, and (iv) the targeting region of the fourth bridging oligonucleotide is complementary to a sequence of a eukaryotic 28S rRNA,

74. The kit of claim 55, wherein the first targeting region of the bridging oligonucleotide comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

75. The kit of claim 55, further comprising a buffer comprising TMAC or TEAC.

76. The kit of claim 55, further comprising a magnetic stand.

77. The kit of claim 55, further comprising:
c) a solid support for preparing a nucleic acid array.

78. A bridging oligonucleotide comprising a (1) bridging region comprising a polypyrimidine or polypurine stretch and a (2) targeting region comprising at least 10 contiguous nucleic acid residues complementary to a sequence of an rRNA.

79. The oligonucleotide of claim 78, wherein the rRNA is eukaryotic.

80. The oligonucleotide of claim 79, wherein the rRNA is the 28S rRNA.

81. The oligonucleotide of claim 78, wherein the rRNA is prokaryotic.

82. The oligonucleotide of claim 81, wherein the rRNA is the 23S rRNA.

83. A method for depleting or isolating a targeted rRNA from a sample comprising:
a) obtaining the kit of claim 55;
b) incubating the sample with the bridging oligonucleotide under conditions allowing hybridization between the targeting region and the targeted rRNA;
c) incubating the bridging oligonucleotide with the capture oligonucleotide under conditions allowing hybridization between the bridging region and the capture region; and
d) isolating the targeted rRNA from the remainder of the sample by incubating the sample with a magnetic field.

84. The method of claim 83, further comprising:
e) obtaining the remainder of the sample enriched for mRNA;
f) preparing cDNA from the mRNA.

85. The method of claim 84, further comprising:
g) constructing a nucleic acid array with the cDNA.
